(19) United States
(12) Patent Application Publication
(10) Pub. No.: US 2011/0081293 A1
(43) Pub. Date: Apr. 7, 2011

(54) METHODS AND COMPOSITIONS RELATED TO CLOT-BINDING LIPID COMPOUNDS

(75) Inventors: ERKKI RUOSLAHTI, BUELLTON, CA (US); DAVID PETERS, VENTURA, CA (US); MATTHEW TIRRELL, Richmond, CA (US); MARK KASTANTIN, BOULDER, CO (US)

(73) Assignees: SANFORD-BURNHAM MEDICAL RESEARCH INSTITUTE, LA JOLLA, CA (US); REGENTS OF THE UNIVERSITY OF CALIFORNIA, OAKLAND, CA (US)

(21) Appl. No.: 12/895,446

(22) Filed: Sep. 30, 2010

Related U.S. Application Data
(60) Provisional application No. 61/249,468, filed on Oct. 7, 2009.

(51) Int. Cl. A61K 38/10; C07K 7/00; C07K 7/08; C07K 5/00; A61K 38/08; A61K 38/06; A61K 49/00; A61K 49/06; A61K 51/00; A61K 9/127; A61P 9/00; A61P 35/00

(52) U.S. Cl. 424/1.73; 530/300; 530/326; 530/330; 530/331; 514/21.8; 514/21.9; 424/9.6; 424/9.3; 424/450; 514/13.7

(57) ABSTRACT

Disclosed are compositions and methods related to clot-binding head groups. The disclosed targeting is useful for treatment of cancer and other diseases and disorders.

### CROSS-REFERENCE TO RELATED APPLICATIONS

This application claims benefit of U.S. Provisional Application No. 61/249,468, filed Oct. 7, 2009. Application No. 61/249,468, filed Oct. 7, 2009, is hereby incorporated herein by reference in its entirety.

### STATEMENT REGARDING FEDERALLY SPONSORED RESEARCH

This invention was made with government support under grant HL070818 awarded by the National Heart, Lung and Blood Institute, grant 1S10RR017753 awarded by the National Center for Research Resources, and DMR05-20415 award by the National Science Foundation. The government has certain rights in this invention.

### BACKGROUND

Cardiovascular disease affects 1 in 3 people in the United States during their lifetime and accounts for nearly a third of the deaths that occur each year (Rosamond W, et al. (2007) Circulation 115, e69-171). Atherosclerosis is one of the leading causes of cardiovascular disease and results in raised plaques in the arterial wall that can occlude the vascular lumen and block blood flow through the vessel. Recently, it has become clear that not all plaques are the same: those susceptible to rupture, fissuring, and subsequent thrombosis are most frequently the cause of acute coronary syndromes and death (Davies M J (1992) Circulation 85, 119-24).

Rupture of an atherosclerotic plaque exposes collagen and other plaque components to the bloodstream. This initiates hemostasis in the blood vessel and leads to activation of thrombin and a thrombus to form at the site of rupture. Elevated levels of activated thrombin bound to the vessel wall have been observed up to 72 hours after vascular injury (Ghigliotti G, (1998) Arterioscler Thromb Vasc Biol 18, 250-257). These elevated thrombin levels not only induce clot formation but also have been implicated in the progression of atherosclerosis by causing smooth muscle cells to bind circulating low density lipoprotein (Ivey M E & Little P J (2008) Thromb Res, 123, 288-297). Subtle clotting in plaques is also indicated by deposition of fibrin/fibrinogen both inside and on the surface of atherosclerotic plaques, which has been well documented since the 1940's (Duguid J B (1948) J Pathol Bacterial 60, 57-61; Smith E B (1993) Wien Klin Wochenschr 105, 417-424; Duguid J B (1946) J Pathol Bacterial 58, 207-212).

Fibrin-containing blood clots have been extensively used as a target for site-specific delivery of imaging agents and anti-clotting agents to thrombi (Bode C, et al., (1994) Circulation 90, 1956-1963; Stoll P., et al., (2007) Arterioscler Thromb Vasc Biol 27, 1206-1212; Alonso A, et al., (2007) Stroke 38, 1508-1514). Delivering anticoagulants into vessels where clotting is taking place has been shown to be effective at reducing the formation and expansion of clots and also decreases the risk of systemic side effects (Bode C, et al., (1994) Circulation 90, 1956-1963; Stoll P., et al., (2007) Arterioscler Thromb Vasc Biol 27, 1206-1212). Antibodies and peptides that bind to molecular markers specifically expressed on atherosclerotic plaques have shown promise for plaque imaging in vivo (Houston P, et al., (2001) FEBS Lett 492, 73-77; Liu C, et al., (2003) Am J Pathol 163, 1859-1871; Kelly K A, et al., (2006) Mol Imaging Biol 8, 201-207; Briley-Saebo K C, et al., (2008) Circulation 117, 3206-3215), however clotting on the plaque has not been used as a target. Fibrin deposited on plaques could serve as a target for delivering diagnostic and therapeutic compounds to plaques.

Nanoparticles containing fibrin homing compounds could be used for delivering diagnostic and therapeutic compounds to plaques. The clot-binding peptide CREKA was identified as a tumor-homing peptide by in vivo phage library screening and subsequently shown to bind to clotted plasma proteins in the blood vessels and stroma of tumors (Simberg D, et al., (2007) Proc Natl Acad Sci USA 104, 932-936; Karmali P P et al., (2009) Nanomedicine, 5, 73-82). CREKA-targeted vehicles can be used to deliver diagnostic and therapeutic compounds to plaques.

### BRIEF SUMMARY

Disclosed are compositions comprising amphiphile molecules, wherein at least one of the amphiphile molecules comprises a clot-binding head group, wherein the clot-binding head group selectively binds to clotted plasma protein, and wherein the composition does not cause clotting.

Also disclosed are methods comprising administering a composition to a subject, wherein the composition comprises amphiphile molecules, wherein at least one of the amphiphile molecules comprises a clot-binding head group, wherein the clot-binding head group selectively binds to clotted plasma protein, wherein the composition does not cause clotting, wherein the composition binds to clotted plasma protein in the subject. Also disclosed are methods comprising administering one or more of the disclosed compositions to a subject, wherein the composition binds to clotted plasma protein in the subject.

Also disclosed are methods of making a composition, the method comprising mixing amphiphile molecules, wherein at least one of the amphiphile molecules comprises a clot-binding head group, wherein the clot-binding head group selectively binds to clotted plasma protein, and wherein the composition does not cause clotting. Also disclosed are methods of making a composition, the method comprising mixing amphiphile molecules, wherein at least one of the amphiphile molecules comprises one or more of the disclosed clot-binding head group.

The amphiphile molecules can comprise a functional head group. At least one of the amphiphile molecules can comprise a functional head group. The functional head group can be a detection head group. The functional head group can be a treatment head group. At least one of the amphiphile molecules can comprise a detection head group and at least one of the amphiphile molecules can comprise a treatment head group.

The amphiphile molecules can be subjected to a hydrophilic medium. The amphiphile molecules can form an aggregate in the hydrophilic medium. The aggregate can comprise a micelle.

The clot-binding head group can comprise amino acid segments independently selected from amino acid segments comprising the amino acid sequence CREKA (SEQ ID NO: 1) or a conservative variant thereof, amino acid segments comprising the amino acid sequence CREKA (SEQ ID NO:1), amino acid segments consisting of the amino acid sequence CREKA (SEQ ID NO:1), amino acid segments consisting of the amino acid sequence REK, or a combination. The amino acid segments each independently can comprise the amino acid sequence CREKA (SEQ ID NO: 1) or a conservative variant thereof. The amino acid segments each independently can comprise the amino acid sequence CREKA (SEQ ID NO:1). At least one of the amino acid segment can consist of the amino acid sequence CREKA (SEQ ID NO:1). At least one of the amino acid segment can consist of the amino acid sequence REK.

The amphiphile molecules can be detectable. The amphiphile molecules can be detectable by fluorescence, PET or MRI. The detection head group can comprise FAM or a derivative thereof.

The treatment head group can comprise a compound or composition for treating cardiovascular disease. The treatment head group can comprise a compound or composition for treating atherosclerosis. The treatment head group can comprise a direct thrombin inhibitor. The treatment head group comprises hirulog or a derivative thereof. The treatment head group can comprise a compound or composition to induce programmed cell death or apoptosis. The treatment head group can comprise a compound or composition for treating cancer. The micelle can comprise the amphiphile molecules. The composition can comprise a liposome, where the liposome comprises the amphiphile molecules.

Also disclosed are conjugates of any of the disclosed compositions and a plaque in a subject. Also disclosed are conjugates of any of the disclosed compositions and a tumor in a subject.

The subject can be in need of treatment of a disease or condition associated with and/or that produces clotted plasma protein. The subject can be in need of treatment of cardiovascular disease. The subject can be in need of detection, visualization, or both of a disease or condition associated with and/or that produces clotted plasma protein. The subject can be in need of detection, visualization, or both of cardiovascular disease. The subject can be in need of detection, visualization, or both of cancer, a tumor, or both. The subject can be in need of treatment of cancer.

Administering the composition can treat a disease or condition associated with and/or that produces clotted plasma protein. Administering the composition can treat a cardiovascular disease. The cardiovascular disease can be atherosclerosis. Administering the composition can treat cancer. The method can further comprise detecting, visualizing, or both the disease or condition associated with and/or that produces clotted plasma protein. The method can further comprise detecting, visualizing, or both the cardiovascular disease. The method can further comprise detecting, visualizing, or both the cancer, tumor, or both.

The method can further comprise, prior to administering, subjecting the amphiphile molecules to a hydrophilic medium. The amphiphile molecules can form an aggregate in the hydrophilic medium. The aggregate can comprise a micelle. The method can further comprise, following administering, detecting the amphiphile molecules. The amphiphile molecules can be detected by fluorescence, PET or MRI. The amphiphile molecules can be detected by fluorescence. The composition can conjugate with a plaque in a subject. The composition can conjugate with a tumor in a subject.

The clot-binding head groups can each be independently selected from an amino acid segment comprising the amino acid sequence REK, a fibrin-binding peptide, a clot-binding antibody, and a clot-binding small organic molecule. The clot-binding head groups can each independently comprise an amino acid segment comprising the amino acid sequence REK.

The clot-binding head groups can each comprise a fibrin-binding peptide. The fibrin-binding peptides can independently be selected from the group consisting of fibrin binding proteins and fibrin-binding derivatives thereof. In another example, the clot-binding head groups can each comprise a clot-binding antibody. Furthermore, the clot-binding head groups can each comprise a clot-binding small organic molecule.

The composition can further comprise a lipid, micelle, liposome, nanoparticle, microparticle, or fluorocarbon microbubble. In one example, the composition can be detectable. In another example, the composition can comprise a treatment head group. An example of a treatment head group is hirulog.

The composition can further comprise one or more head groups. For example, the head groups can be independently selected from the group consisting of an anti-angiogenic agent, a pro-angiogenic agent, a cancer chemotherapeutic agent, a cytotoxic agent, an anti-inflammatory agent, an anti-arthritic agent, a polypeptide, a nucleic acid molecule, a small molecule, an image contrast agent, a fluorophore, fluorescein, rhodamine, a radionuclide, indium-111, technetium-99, carbon-11, and carbon-13. At least one of the head groups can be a treatment head group. Examples of treatment head groups are paclitaxel and taxol. At least one of the head groups can be a detection head group.

The composition can selectively home to clotted plasma protein. The composition can selectively home to tumor vasculature, wound sites, or both. In one example, the composition can have a therapeutic effect. This effect can be enhanced by the delivery of a treatment head group to the site of the tumor or wound site.

The therapeutic effect can be a slowing in the increase of or a reduction of cardiovascular disease. The therapeutic effect can be a slowing in the increase of or a reduction of atherosclerosis. The therapeutic effect can be a slowing in the increase of or a reduction of the number and/or size of plaques. The therapeutic effect can be a reduction in the level or amount of the causes or symptoms of the disease being treated. The therapeutic effect can be a slowing in the increase of or a reduction of tumor burden.

The subject can have one or more sites to be targeted, wherein the composition homes to one or more of the sites to be targeted. For example, the subject can have multiple tumors or sites of injury.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows tumor homing of CREKA pentapeptide. Fluorescein-conjugated CREKA peptide (200 μg per mouse) was injected into mice bearing syngeneic B16 melanoma tumors. Representative microscopic fields are shown to illustrate homing of fluorescein-CREKA to fibrin-like structures in tumors in wild type mice (A, arrow) and lack of homing in fibrinogen null mice (B). (C) The CREKA phage binds to clotted plasma proteins in the tube, while non-recombinant control phage shows little binding. (D) Dextran-coated iron oxide nanoparticles conjugated with fluorescein-CREKA bind to clotted plasma proteins, and the binding is inhibited by free CREKA peptide. The inset in (D) shows the microscopic appearance of the clot-bound CREKA-SPIO. Magnification: A-B, 200×; D, 600×.

FIG. 2 shows tumor homing of CREKA-conjugated iron oxide particles. CREKA-SPIO particles were intravenously injected (4 mg Fe/kg) into Balb/c nude mice bearing MDA-MB-435 human breast cancer xenograft tumors measuring 1-1.5 cm in diameter. The mice were sacrificed by perfusion 5-6 hours later and tissues were examined for CREKA-SPIO fluorescence (green). Nuclei were stained with DAPI (blue). (A) Distribution of CREKA-SPIO in tissues from MDA-MB-435 tumor mice that received 2 hours earlier an injection of PBS (A, upper panels) or Ni/DSPC/CHOL liposomes (Ni-liposomes) containing 0.2 μmol Ni in 200 μl of PBS (A, lower panels). (B) Plasma circulation half-life of CREKA-SPIO following different treatments. At least 4 time points were collected. Data were fitted to mono-exponential decay using Prizm software (GraphPad, San Diego, Calif.), and the half-life values were compared in unpaired t-test (***p<0.0001, n=10). (C) Accumulation of CREKA-SPIO nanoparticles in tumor vessels. Mice were injected and tissues collected as in panel A. Fluorescent intravascular CREKA-SPIO particles overlap with iron oxide viewed in transmitted light. Magnification: 600×. (D) Control organs of Ni-liposome/CREKA-SPIO-injected mice. Occasional spots of fluorescence are seen in the kidneys and lungs. The fluorescence seen in the heart did not differ from uninjected controls, indicating that it is autofluorescence. Representative results from at least 3 independent experiments are shown. Magnification A and D, 200×; C, 600×.

FIG. 3 shows the accumulation of CREKA-SPIO nanoparticles in tumor vessels. Mice bearing MDA-MB-435 xenografts were injected with Ni-liposomes and CREKA-SPIO nanoparticles as described in the legend to FIG. 2. The mice were perfused 6 hours after the nanoparticle injection and tissues were collected. (A) Upper panels: Co-localization of nanoparticle fluorescence with CD31 staining in blood vessels; Middle panels: Co-localization of nanoparticle fluorescence and anti-fibrin(ogen) staining in tumor blood vessels. Inset—an image showing CREKA-SPIO distributed along fibrils in a tumor blood vessel; Lower panels: Lack of co-localization of nanoparticle fluorescence with anti-CD41 staining for platelets. (B) Intravital confocal microscopy of tumors using DiI-stained red blood cells as a marker of blood flow. The arrow points to a vessel in which stationary erythrocytes indicate obstruction of blood flow. Blood flow in the vessel above is not obstructed. Six successive frames from a 1-min movie (Movie 2 in Supplementary Material) are shown. (C) CREKA-coated liposomes co-localize with fibrin in tumor vessels. The results are representative of 3 independent experiments. Magnification: A and C, 600×, B, 200×.

FIG. 4 shows the effect of blood clotting on nanoparticle accumulation in tumors. Mice bearing MDA-MB-435 human breast cancer xenografts were intravenously injected with PBS or a bolus of 800 U/kg of heparin followed 120 min later by Ni-liposomes (or PBS) and CREKA-SPIO (or control nanoparticles). The mice received additional heparin by intraperitoneal injections (a total of 1000 U/kg) or PBS throughout the experiment. (A) Tumors were removed 6 hours after the nanoparticle injection, and magnetic signal in the tumor after different treatments was determined with SQUID. Aminated dextran SPIO served as a particle control (control SPIO). SPIO nanoparticle concentration in tissues is represented by the saturation magnetization value (electromagnetic unit, emu) of the tissue at 1T magnetic field after the subtraction of the diamagnetic and the paramagnetic background of blank tissue. The magnetization values were normalized to dry weight of the tissue. Results from 3 experiments are shown. (B) Quantification of heparin effect on clotting in blood vessels. Mice were pretreated with PBS (white bars) or heparin (black bars) as described above, followed by Ni liposomes/CREKA-SPIO nanoparticles. Three sections from two tumors representing each treatment were stained with anti-CD31 for blood vessels, and the percentage of vessels positive for fluorescence and fluorescent clots was determined. Note that heparin did not significantly change the percentage of blood vessels containing particles, but dramatically decreased the incidence of the lumens that are filled with fluorescence. (C) A representative example of the appearance of CREKA-SPIO particles in tumor vessels of mice treated with heparin. (D) Near-infrared imaging of mice that received Ni-liposomes followed by Cy7-labeled CREKA-SPIO with or without heparin pretreatment. The images were acquired 8 hours after the injection of the CREKA-SPIO particles using an Odyssey 2 NIR scanner (Li-COR Biosciences, Lincoln, Neb.). The images shown are composites of 2 colors, red (700 nm channel, body and chow autofluorescence) and green (800 nm channel, Cy7). Arrows point to the tumors, arrowheads to the liver. Note the strong decrease in signal from the tumor in the heparin-pretreated mouse. A representative experiment out of 3 is shown.

FIG. 5 shows tumor homing of CREKA peptide. (A). Balb/c nude mice bearing MDA-MB-435 human breast cancer xenograft tumors or transgenic MMTV PyMT mice with breast tumors were intravenously injected with 0.1 mg of fluorescein-CREKA. The animals were sacrificed by perfusion 24 hours post-injection and tissue sections were examined by fluorescent microscopy. Right panel, control organs of MDA-MB 435 tumor mice. Magnification 200×. (B). Whole animal imaging of MDA-MB-435 tumor mouse injected 6 hours earlier with 30 μg of Alexa Fluor 647-labeled CREKA. Maestro imaging system (Cambridge Research Inc., Woburn, Mass.) was used to acquire and process the image. The arrow points to the tumor and the arrowhead to the urinary bladder. Note that the peptide is excreted into the urine and does not accumulate in the liver.

FIG. 6 shows fluorescence intensity of iron oxide nanoparticles (CREKA-SPIO) coupled to various levels of substitution with fluorescein-labeled CREKA peptide. Fluorescence emitted by the conjugated particles is linearly related to the level of substitution. A.U.=Arbitrary Units.

FIG. 7 shows CREKA-SPIO nanoparticles accumulate in tumor tissue, but not in non-RES normal tissues. The low magnification (40×) was used to produce these images because only blood vessels in which clotting had concentrated the CREKA-SPIO fluorescence are visible at this magnification. Note the entrapment of nanoparticles in clots in tumor tissue (arrow), but not in non-RES normal tissues. The injections were carried out and the tissues prepared for analysis as in FIG. 2. A representative experiment out of 10 is shown.

FIG. 8 shows lack of colocalization of fibrin(ogen) staining and CREKA-SPIO in the liver. The fibrin(ogen)-positive structures can be background from fibrinogen production by the liver, as it does not co-localize with the nanoparticles (A), and the liver from a non-injected mouse showed similar fibrin(ogen) staining (B). Magnification 600×.

FIG. 9 shows the role of platelets in nanoparticle homing. (A). Blood was drawn 5 min post-injection of 4 mg/kg of CREKA-SPIO into mice and a 50 μl aliquot was run through a magnetic column. Bound CREKA-SPIO particles were eluted form the column, concentrated on a slide, and stained with anti-CD41 antibody. Some of the particles appear to be associated with platelets. (B). A low-magnification image (40x) showing CREKA-SPIO homing and clot formation in a tumor from a platelet-depleted mouse. Platelet depletion was accomplished by treating mice with 0.1 mg of an anti-CD41 monoclonal antibody as described (Van der Heyde and Gramaglia (2005)). The mice subsequently received Ni-liposomes/CREKA-SPIO as described in the legend of FIG. 2. The anti-platelet treatment did not decrease the incidence of fluorescent clots (compare with the tumor panel in FIG. 7).

FIG. 10 shows the construction of modular, multifunctional micelles. (A) Individual lipopeptide monomers are made up of a 1,2-distearoyl-sn-glycero-3-phosphoethanol-amine (DSPE) tail, a polyethyleneglycol (PEG2000) spacer, and a variable polar headgroup that contains either CREKA, FAM-CREKA, FAM, N-acetyl-cysteine, Cy7, or hirulog. The monomers were combined to form various mixed micelles. (B) Three dimensional structure of FAM-CREKA/Cy7/hirulog mixed micelle.

FIG. 11 shows the ex vivo imaging of the aortic tree of atherosclerotic mice. Micelles were injected intravenously and allowed to circulate for three hours. The aortic tree was excised following perfusion and imaged ex vivo. (A) Increased fluorescence was observed in the aortic tree of ApoE null mice following injection with FAM-CREKA targeted micelles but not with non-targeted fluorescent micelles. When an excess of unlabeled CREKA micelles was injected prior to the FAM-CREKA micelles, fluorescence in the aortic tree was decreased. A pre-injection of an excess of non-targeted, unlabeled micelles did not cause a significant decrease in fluorescence. (B) Fluorescence in the aortic tree was quantified by measuring the intensity of fluorescent pixels (n=3 per group).

FIG. 12 shows the localization of CREKA micelles in atherosclerotic plaques. (A) Serial cross-sections (5 μm thick) were stained with antibodies against CD31 (endothelial cells), CD68 (macrophages and other lymphocytes), and fibrinogen. Representative microscopic fields are shown to illustrate the localization of micelle nanoparticles in the atherosclerotic plaque. Micelles are bound to the entire surface of the plaque with no apparent binding to the healthy portion of the vessel. CREKA targeted micelles also penetrate under the endothelial layer (CD31 staining) in the shoulder of the plaque (inset) where there is high inflammation (CD68 staining) and the plaque is prone to rupture. Clotted plasma proteins are seen throughout the plaque and it surface (fibrinogen staining). Images in the left panels were taken at a 10× magnification (bar=200 μm) and images in the right panel are taken at a 150× magnification (bar=20 μm). (B) Fluorescence was not observed in the heart or lung, and only a small amount was seen in the kidney, spleen, and liver. Images were taken at a 20× magnification (bar=100 μm).

FIG. 13 shows the specific targeting of hirulog to atherosclerotic plaques. (A) Equal molar concentrations of hirulog peptide and hirulog micelles were tested for anti-thrombin activity to ensure that potency did not decrease when hirulog was in micellar form. Hirulog peptide and micelles showed similar activity in a chromogenic assay. (B) CREKA targeted or non-targeted, hirulog mixed micelles were injected intravenously into mice and allowed to circulate for 3 hours. The aortic tree was excised and analyzed for bound hirulog. Significantly higher levels of anti-thrombin activity were observed in the aortic tree of ApoE null mice following injection of CREKA targeted hirulog micelles than non-targeted micelles (1.8 μg/mg and 1.2 μg/mg of tissue, p≦0.05, n=3 per group). Anti-thrombin activity generated by CREKA targeted hirulog micelles in ApoE null mice was also significantly higher than that in wild-type mice (0.8 μg/mg of tissue, p<0.05, n=3 per group).

FIG. 14 shows the specific targeting micelles to atherosclerotic plaques. ApoE null and wild-type mice were injected intravenously with FAM-CREKA micelles, which were allowed to circulate for 3 hours. (A, C) The aortic tree was excised following perfusion and imaged ex vivo. (B, D) Histological cross-sections were also analyzed for binding of micelles to the vessel wall. Higher fluorescence intensity was observed in (C) ApoE mice relative to (A) wild-type mice with ex vivo imaging. Fluorescent CREKA micelles did not bind to the healthy vessels in the histological sections of (B) wild-type mice but were observed on the surface of the atherosclerotic lesions in the (D) ApoE null mice. Histological images were taken at 10× magnification (bar=200 μm).

FIG. 15 shows the role of clotting in binding of CREKA micelles. (A) Mice were injected intravenously with PBS or a bolus of 800 units/kg of heparin, followed 60 minutes later by 100 μl of 1 mM FAM-CREKA micelles. The mice received additional heparin (a total of 1,000 units/kg) or PBS throughout the experiment. Similar fluorescence was observed in the aortic tree of ApoE null mice that received a pre-injection of PBS or heparin followed by an injection of FAM-CREKA micelles. (B) CREKA micelles did not induce clotting in 22RV1 mouse prostate tumor model. Sections 5 μm thick were stained with antibodies against fibrinogen. Representative microscopic fields are shown to illustrate that FAM-CREKA micelles bind to the blood vessels in the tumor but do not cause fibrin clots to form. Images were taken at 40× magnification (bar=50 μm).

FIG. 16 is an illustration of surface-based method for producing liposomes using amphiphile molecules.

### DETAILED DESCRIPTION

The disclosed method and compositions can be understood more readily by reference to the following detailed description of particular embodiments and the Example included therein and to the Figures and their previous and following description.

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Materials

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular peptide is disclosed and discussed and a number of modifications that can be made to a number of molecules including the peptide are discussed, specifically contemplated is each and every combination and permutation of the peptides and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Disclosed are compositions comprising amphiphile molecules, wherein at least one of the amphiphile molecules comprises a clot-binding head group, wherein the clot-binding head group selectively binds to clotted plasma protein, and wherein the composition does not cause clotting.

Also disclosed are methods comprising administering a composition to a subject, wherein the composition comprises amphiphile molecules, wherein at least one of the amphiphile molecules comprises a clot-binding head group, wherein the clot-binding head group selectively binds to clotted plasma protein, wherein the composition does not cause clotting, wherein the composition binds to clotted plasma protein in the subject. Also disclosed are methods comprising administering one or more of the disclosed compositions to a subject, wherein the composition binds to clotted plasma protein in the subject.

Also disclosed are methods of making a composition, the method comprising mixing amphiphile molecules, wherein at least one of the amphiphile molecules comprises a clot-binding head group, wherein the clot-binding head group selectively binds to clotted plasma protein, and wherein the composition does not cause clotting. Also disclosed are methods of making a composition, the method comprising mixing amphiphile molecules, wherein at least one of the amphiphile molecules comprises one or more of the disclosed clot-binding head group.

The amphiphile molecules can comprise a functional head group. At least one of the amphiphile molecules can comprise a functional head group. The functional head group can be a detection head group. The functional head group can be a treatment head group. At least one of the amphiphile molecules can comprise a detection head group and at least one of the amphiphile molecules can comprise a treatment head group.

The amphiphile molecules can be subjected to a hydrophilic medium. The amphiphile molecules can form an aggregate in the hydrophilic medium. The aggregate can comprise a micelle.

The clot-binding head group can comprise amino acid segments independently selected from amino acid segments comprising the amino acid sequence CREKA (SEQ ID NO: 1) or a conservative variant thereof, amino acid segments comprising the amino acid sequence CREKA (SEQ ID NO:1), amino acid segments consisting of the amino acid sequence CREKA (SEQ ID NO:1), amino acid segments consisting of the amino acid sequence REK, or a combination. The amino acid segments each independently can comprise the amino acid sequence CREKA (SEQ ID NO: 1) or a conservative variant thereof. The amino acid segments each independently can comprise the amino acid sequence CREKA (SEQ ID NO:1). At least one of the amino acid segment can consist of the amino acid sequence CREKA (SEQ ID NO:1). At least one of the amino acid segment can consist of the amino acid sequence REK.

The clot-binding head groups can each be independently selected from, for example, an amino acid segment comprising the amino acid sequence REK, a fibrin-binding peptide, a peptide that binds clots and not fibrin (such as CGLIIQKNEC (CLT1, SEQ ID NO: 2) and CNAGESSKNC (CLT2, SEQ ID NO: 3)). a clot-binding antibody, and a clot-binding small organic molecule.

The amphiphile molecules can be detectable. The amphiphile molecules can be detectable by fluorescence, PET or MRI. The amphiphile molecules can be detectable by fluorescence. The detection head group can comprise FAM or a derivative thereof.

The treatment head group can comprise a compound or composition for treating cardiovascular disease. The treatment head group can comprise a compound or composition for treating atherosclerosis. The treatment head group can comprise a direct thrombin inhibitor. The treatment head group comprises hirulog or a derivative thereof. The treatment head group can comprise a compound or composition for treating cancer. The micelle can comprise the amphiphile molecules. The composition can comprise a liposome, where the liposome comprises the amphiphile molecules.

Also disclosed are conjugates of any of the disclosed compositions and a plaque in a subject. Also disclosed are conjugates of any of the disclosed compositions and a tumor in a subject.

The subject can be in need of treatment of a disease or condition associated with and/or that produces clotted plasma protein. The subject can be in need of treatment of cardiovascular disease. The subject can be in need of detection, visualization, or both of a disease or condition associated with and/or that produces clotted plasma protein. The subject can be in need of detection, visualization, or both of cardiovascular disease. The subject can be in need of detection, visualization, or both of cancer, a tumor, or both. The subject can be in need of treatment of cancer. By “a disease or condition associated with clotted plasma protein” is meant that the disease or condition that causes production and/or formation of clotted plasma protein, that causes production and/or formation of blood clots, that causes production and/or formation of atherosclerotic plaques, that has as a symptom clotted plasma protein, that has as a symptom blot clots, that has as a symptom atherosclerotic plaques, that is caused by clotted plasma protein, that is caused by blood clots, that is caused by atherosclerotic plaques, that is characterized by clotted plasma protein, that is characterized by blood clots, that is characterized by atherosclerotic plaques, the symptoms of which are worsened by clotted plasma protein, the symptoms of which are worsened by blood clots, the symptoms of which are worsened by atherosclerotic plaques, or a combination.

Administering the composition can treat a disease or condition associated with and/or that produces clotted plasma protein. Administering the composition can treat a cardiovascular disease. The cardiovascular disease can be atherosclerosis. Administering the composition can treat cancer. The method can further comprise detecting, visualizing, or both the disease or condition associated with and/or that produces clotted plasma protein. The method can further comprise detecting, visualizing, or both the cardiovascular disease. The method can further comprise detecting, visualizing, or both the cancer, tumor, or both.

The method can further comprise, prior to administering, subjecting the amphiphile molecules to a hydrophilic medium. The amphiphile molecules can form an aggregate in the hydrophilic medium. The aggregate can comprise a micelle. The method can further comprise, following administering, detecting the amphiphile molecules. The amphiphile molecules can be detected by fluorescence, CT scan, PET or MRI. The amphiphile molecules can be detected by fluorescence. The composition can conjugate with a plaque in a subject. The composition can conjugate with a tumor in a subject.

Disclosed herein is a composition comprising a amphiphile molecule and a clot-binding head group. The clot-binding head groups can selectively bind to clotted plasma protein. In some forms, the composition does not cause or enhance clotting.

A number of appropriate clot-binding head groups have been identified that are specifically or preferentially expressed, localized, adsorbed to or inducible on cells or in the clotted blood proteins. These are discussed in more detail below.

### A. Amphiphile Molecules

Amphiphile molecules, alternatively referred to as amphiphiles or amphiphilic molecules, are any substance that can form monolayers, vesicles, micelles, bilayers, liposomes, and the like when in aqueous environments. Amphiphile molecules are amphiphilic and comprise one or more hydrophobic groups and one or more hydrophilic groups. The hydrophobic groups can be referred to as the tail of the amphiphile molecule and the hydrophilic groups can be referred to as the head of the amphiphile molecule. Useful amphiphile molecules include surfactants, fatty acids, lipids, sterols, monoglycerides, diglycerides, triglycerides (fats), phospholipids, glycerolipids, glycerophospholipids, sphingolipids, sterol lipids, prenol lipids, saccharolipids, polyketides, block copolymers, combinations, and the like. The disclosed amphiphile molecules can be ionic, anionic, cationic, zwitterionic, and nonionic.

The term amphiphile molecule is not intended to be limiting. In particular, the disclosed amphiphile molecules are not limited to substances, compounds, compositions, particles or other materials composed of a single molecule. Rather, the disclosed amphiphile molecules can be any substance(s), compound(s), composition(s), particle(s) and/or other material(s) that is amphiphilic can be used with and in the disclosed compositions and methods.

Amphiphilic molecules have two distinct components, differing in their affinity for a solute, most particularly water. The part of the molecule that has an affinity for water, a polar solute, is said to be hydrophilic. The part of the molecule that has an affinity for non-polar solutes such as hydrocarbons is said to be hydrophobic. When amphiphilic molecules are placed in water, the hydrophilic moiety seeks to interact with the water while the hydrophobic moiety seeks to avoid the water. To accomplish this, the hydrophilic moiety remains in the water while the hydrophobic moiety is held above the surface of the water in the air or in a non-polar, non-miscible liquid floating on the water. The presence of this layer of molecules at the water's surface disrupts the cohesive energy at the surface and lowers surface tension. Amphiphilic molecules that have this effect are known as amphiphiles. Only so many amphiphiles can align as just described at the water/air or water/hydrocarbon interface. A variety of examples of suitable amphiphiles are described and disclosed herein.

1. Lipids

Lipids are synthetically or naturally-occurring molecules which includes fats, waxes, sterols, prenol lipids, fat-soluble vitamins (such as vitamins A, D, E and K), glycerolipids, monoglycerides, diglycerides, triglycerides, glycerophospholipids, sphingolipids, phospholipids, fatty acids monoglycerides, saccharolipids and others. Lipids can be hydrophobic or amphiphilic small molecules; the amphiphilic nature of some lipids allows them to form structures such as monolayers, vesicles, micelles, liposomes, bi-layers or membranes in an appropriate environment i.e. aqueous environment. Any of a number of lipids can be used as amphiphile molecules, including amphipathic, neutral, cationic, and anionic lipids. Such lipids can be used alone or in combination, and can also include bilayer stabilizing components such as polyamide oligomers (see, e.g., U.S. Pat. No. 6,320,017, “Polyamide Oligomers”, by Ansell), peptides, proteins, detergents, lipid-derivatives, such as PEG coupled to phosphatidylethanolamine and PEG conjugated to ceramides (see, U.S. Pat. No. 5,885,613). In a preferred embodiment, cloaking agents, which reduce elimination of liposomes by the host immune system, can also be included, such as polyamide-oligomer conjugates, e.g., ATTA-lipids, (see, U.S. patent application Ser. No. 08/996,783, filed Feb. 2, 1998) and PEG-lipid conjugates (see, U.S. Pat. Nos. 5,820,873, 5,534,499 and 5,885,613).

Any of a number of neutral lipids can be included, referring to any of a number of lipid species which exist either in an uncharged or neutral zwitterionic form at physiological pH, including diacylphosphatidylcholine, diacylphosphatidylethanolamine, ceramide, sphingomyelin, cephalin, cholesterol, cerebrosides, and diacylglycerols.

Cationic lipids, carry a net positive charge at physiological pH, can readily be used as amphiphile molecules. Such lipids include, but are not limited to, N,N-dioleyl-N,N-dimethylammonium chloride (“DODAC”); N-(2,3-dioleyloxy) propyl-N,N-N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 3.beta.-(N-(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethyl-ammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), and N-(1,2-dimyristyloxyprop-3-yl)-N,N-dimethyl-N-hydroxyethyl ammonium bromide (“DMRIE”). Additionally, a number of commercial preparations of cationic lipids can be used, such as LIPOFECTIN (including DOTMA and DOPE, available from GIBCO/BRL), LIPOFECTAMINE (comprising DOSPA and DOPE, available from GIBCO/BRL), and TRANSFECTAM (comprising DOGS, in ethanol, from Promega Corp.).

Anionic lipids can be used as amphiphile molecules and include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

Amphiphatic lipids can also be suitable amphiphile molecules. “Amphipathic lipids” refer to any suitable material, wherein the hydrophobic portion of the lipid material orients into a hydrophobic phase, while the hydrophilic portion orients toward the aqueous phase. Such compounds include, but are not limited to, fatty acids, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleoylphosphatidylcholine. Other phosphorus-lacking compounds, such as sphingolipids, glycosphingolipid families, diacylglycerols, and β-acyloxyacids, can also be used. Additionally, such amphipathic lipids can be readily mixed with other lipids, such as triglycerides and sterols. Zwitterionic lipids are a form of amphiphatic lipid.

Sphingolipids are fatty acids conjugated to the aliphatic amino alcohol sphingosine. The fatty acid can be covalently bond to sphingosine via an amide bond. Any amino acid as described above can be covalently bond to sphingosine to form a sphingolipid. A sphingolipid can be further modified by covalent bonding through the α-hydroxyl group. The modification can include alkyl groups, alkenyl groups, alkynyl groups, aromatic groups, heteroaromatic groups, cyclyl groups, heterocyclyl groups, phosphonic acid groups. Non-limiting examples of shingolipids are N-acylsphingosine, N-Acylsphingomyelin, Forssman antigen.

Saccharolipids are compounds that contain both fatty acids and sugars. The fatty acids are covalently bonded to a sugar backbone. The sugar backbone can contain one or more sugars. The fatty acids can bond to the sugars via either amide or ester bonds. The sugar can be any sugar base. The fatty acid can be any fatty acid as described elsewhere herein. The provided compositions can comprise either natural or synthetic saccharolipids. Non-limiting saccharolipids are UDP-3-O-(β-hydroxymyristoyl)-GlcNAc, lipid IV A, Kdo2-lipid A.

i. Fatty Acids

Fatty acids are aliphatic monocarboxylic acids derived from, or contained in esterified form in, an animal or vegetable fat, oil, or wax. Fatty acids can be synthetic or natural. Natural fatty acids commonly have a chain of four to 28 carbons (usually unbranched and even numbered), which can be saturated or unsaturated. “Fatty acids” is used to include all acyclic aliphatic carboxylic acids.

Fatty acids can be conjugated to the provided compositions include those that allow the efficient incorporation of the proprotein convertase inhibitors into liposomes. Generally, the fatty acid is a polar lipid. The fatty acid can be a free fatty acid (palmitic acid or palmitoleic acid are examples). The composition can comprise either natural or synthetic fatty acids. The fatty acid can be branched or unbranched and saturated or unsaturated. Non-limiting examples of fatty acids are butyric acid, valeric acid, caproic acid, caprylic acid, pelargonic acid, capric acid, lauric acid, myristic acid, palmitic acid, margaric (daturic) acid, stearic acid, arachidic acid, behenic acid, lignoceric acid, cerotic acid, carboceric acid, montanic acid, melissic acid, lacceroic acid, ceromelissic (psyllic) acid, geddic acid, ceroplastic acid, caproleic acid, lauroleic acid, linderic acid, myristoleic acid, physeteric acid, tsuzuic acid, palmitoleic acid, sapienic acid, petroselinic acid, oleic acid, elaidic acid, vaccenic (asclepic) acid, gadoleic acid, gondoic acid, cetoleic acid, erucic acid, nervonic acid, linoleic acid, γ-linolenic acid, dihomo-γ-linolenic acid, arachidonic acid, α-linolenic acid, stearicdonic acid, EPA, DPA, DHA, nisinic acid, mead acid. These tail of these fatty acids can also be modified to include for example alkyl groups, alkenyl groups, alkynyl groups, aromatic groups, heteroaromatic groups, cyclyl groups, heterocyclyl groups, hydroxyl groups, keto groups, acid groups, amine groups, amide groups, phosphor groups or sulfur groups.

A fatty acid can be conjugated to a glycerol. One, two or three fatty acids can be conjugated to a glycol molecule.

A monoglycerides or monoacylglycerol consists of one fatty acid chain covalently bonded to a glycerol molecule through an ester linkage. Monoacylglycerol can either be 1-monoacylglycerols or 2-monoacylglycerols, depending on the position of the ester bond on the glycerol moiety. Monoacylglycerol can contain any of the above described fatty acids as either 1-monoacylglycerols or 2-monoacylglycerols.

A diglyceride, or a diacylglycerol, is a glyceride consisting of two fatty acid chains covalently bonded to a glycerol molecule through ester linkages. Diacylglycerols can have any combinations of fatty acids described above at both the C-1 and C-2 positions. One example is 1-palmitoyl-2-oleoyl-glycerol, which contains side-chains derived from palmitic acid and oleic acid.

A triglyceride or triacylglycerol is a glyceride in which the glycerol is covalently bonded to three fatty acids through ester linkages. Triglycerides can contain any combination of the above described fatty acids in any order. One example is the when glycerol is bonded to palmitic acid, oleic acid and stearic acid in that order.

The fatty acid can be conjugated to another moiety i.e. phospholipids or shingolipids. The fatty acid can be conjugated to phosphonic acid, i.e. phospholipids. Phospholipids can either be sphingolipids or phosphoglycerides. Phosphoglycerides are glycerol based phospholipids. For instance, diglyceride is further conjugated to phosphonic acid through glycerol i.e. glycerophospholipids. Thus, the fatty acid can be conjugated to other polar groups to form lipids i.e. phospholipid. The phospholipids can be water soluble or miscible phospholipids. Non-limiting examples are glycerophosphates, glycerophosphorylcholines, phosphorylcholines, glycerophosphorylethanolamines, phosphoryl-ethanolamines, ethanolamines, glycerophosphorylserines, and glycerophosphosphorylglycerols. The provided compositions can comprise either natural or synthetic phospholipid. The phospholipids can be selected from phospholipids containing saturated or unsaturated mono or disubstituted fatty acids and combinations thereof. These phospholipids can be dioleoylphosphatidylcholine, dioleoylphosphatidylserine, dioleoylphosphatidylethanolamine, dioleoylphosphatidylglycerol, dioleoylphosphatidic acid, palmitoyloleoylphosphatidylcholine, palmitoyloleoylphosphatidylserine, palmitoyloleoylphosphatidylethanolamine, palmitoyloleoylphophatidylglycerol, palmitoyloleoylphosphatidic acid, palmitelaidoyloleoylphosphatidylcholine, palmitelaidoyloleoylphosphatidylserine, palmitelaidoyloleoylphosphatidylethanolamine, palmitelaidoyloleoylphosphatidylglycerol, palmitelaidoyloleoylphosphatidic acid, myristoleoyloleoylphosphatidylcholine, myristoleoyloleoylphosphatidylserine, myristoleoyloleoylphosphatidylethanoamine, myristoleoyloleoylphosphatidylglycerol, myristoleoyloleoylphosphatidic acid, dilinoleoylphosphatidylcholine, dilinoleoylphosphatidylserine, dilinoleoylphosphatidylethanolamine, dilinoleoylphosphatidylglycerol, dilinoleoylphosphatidic acid, palmiticlinoleoylphosphatidylcholine, palmiticlinoleoylphosphatidylserine, palmiticlinoleoylphosphatidylethanolamine, palmiticlinoleoylphosphatidylglycerol, palmiticlinoleoylphosphatidic acid. These phospholipids may also be the monoacylated derivatives of phosphatidylcholine (lysophophatidylidylcholine), phosphatidylserine (lysophosphatidylserine), phosphatidylethanolamine (lysophosphatidylethanolamine), phophatidylglycerol (lysophosphatidylglycerol) and phosphatidic acid (lysophosphatidic acid). The monoacyl chain in these lysophosphatidyl derivatives may be palimtoyl, oleoyl, palmitoleoyl, linoleoyl myristoyl or myristoleoyl. The phospholipids can also be synthetic. Synthetic phospholipids are readily available commercially from various sources, such as AVANTI Polar Lipids (Albaster, Ala.); Sigma Chemical Company (St. Louis, Mo.). These synthetic compounds may be varied and may have variations in their fatty acid side chains not found in naturally occurring phospholipids. The fatty acid can have unsaturated fatty acid side chains with C14, C16, C18 or C20 chains length in either or both the PS or PC. Synthetic phospholipids can have dioleoyl (18:1)-PS; palmitoyl (16:0)-oleoyl (18:1)-PS, dimyristoyl (14:0)-PS; dipalmitoleoyl (16:1)-PC, dipalmitoyl (16:0)-PC, dioleoyl (18:1)-PC, palmitoyl (16:0)-oleoyl (18:1)-PC, and myristoyl (14:0)-oleoyl (18:1)-PC as constituents. Thus, as an example, the provided compositions can comprise palmitoyl 16:0.

ii. Prenols

Prenol lipids are naturally synthesized from the 5-carbon precursors isopentenyl diphosphate and dimethylallyl diphosphate that are produced mainly via the mevalonic acid (MVA) pathway. Prenols can also be made synthetically. The simple isoprenoids (linear alcohols, diphosphates, etc.) are formed by the successive addition of C5 units, and are classified according to number of these terpene units. Structures containing greater than 40 carbons are known as polyterpenes. Carotenoids are important simple isoprenoids that function as antioxidants and as precursors of vitamin A. Prokaryotes synthesize polyprenols (called bactoprenols) in which the terminal isoprenoid unit attached to oxygen remains unsaturated, whereas in animal polyprenols (dolichols) the terminal isoprenoid is reduced. Non-limiting examples of prenols are nerol, catalpol, menthol, neomenthol, perillyl alcohol, carvacrol.

iii. Sterols

Sterols are lipids. Sterols have a 4-cyclic sterane structure that can be modified. Sterol contain one or more hydroxyl groups on the sterane structure. One hydroxyl group can be in the 3 position of the sterane structure. Sterols can be further modified by substituting one or more hydrogen atoms for a range of functional groups. The functional groups include but are not limited to alkyl groups, alkenyl groups, alkynyl groups, aromatic groups, heteroaromatic groups, cyclyl groups, heterocyclyl groups, hydroxyl groups, keto groups, acid groups, amine groups, amide groups, phosphor groups or sulfur groups. The sterols can either be natural or synthetic. Non-limiting examples of sterols are cholesterol, phytosterol, ergosterol, sitosterol, campesterol, stigmasterol, spinosterol, taraxasterol, brassicasterol, desmosterol, chalinosterol, poriferasterol, and clionasterol.

iv. Polyketides

Polyketides are a large, structurally diverse family of compounds. Polyketides possess a broad range of biological activities including antibiotic and pharmacological properties. For example, polyketides are represented by such antibiotics as tetracyclines and erythromycin, anticancer agents including daunomycin, immunosuppressants, for example FK506 and rapamycin, and veterinary products such as monensin and avermectin. Polyketides occur in most groups of organisms and are especially abundant in a class of mycelial bacteria, the actinomycetes, which produce various polyketides. Non-limiting examples of polyketides are trichostatin, tautomycetin, laurenenyne A, tylosin, spiramycin.

2. Block Copolymers

Block copolymers are copolymers that contain two or more differing polymer blocks selected, for example, from homopolymer blocks, copolymer blocks (e.g., random copolymer blocks, statistical copolymer blocks, gradient copolymer blocks, periodic copolymer blocks), and combinations of homopolymer and copolymer blocks. A polymer “block” refers to a grouping of multiple copies of a single type (homopolymer block) or multiple types (copolymer block) of constitutional units. A “chain” is an unbranched polymer block. A polymer block can be a grouping of at least two (e.g., at least five, at least 10, at least 20, at least 50, at least 100, at least 250, at least 500, at least 750) and/or at most 1000 (e.g., at most 750, at most 500, at most 250, at most 100, at most 50, at most 20, at most 10, at most five) copies of a single type or multiple types of constitutional units. A polymer block may take on any of a number of different architectures.

The X-(AB)ₙ structures are frequently referred to as diblock copolymers (when n=1) or triblock copolymers (when n=2). (This terminology disregards the presence of the initiator, for example, treating A-X-A as a single A block with the triblock therefore denoted as BAB.) Where n=3 or more, these structures are commonly referred to as star-shaped block copolymers.

The segments A and B are linked together through a bond that is non-hydrolyzable. A non-hydrolyzable bond is a covalent bond that is insignificantly cleaved by an ordinary aqueous or solvent hydrolysis reaction, e.g. at pH between about 6 and about 8. Specific bonds that are non-hydrolyzable are known to those skilled in the art and include amides, esters, ethers and the like.

A non-hydrolyzable bond between segments A and B in the amphiphilic segmented copolymer can be formed by polymerizing a suitable hydrophilic monomer in the presence of a suitably functionalized hydrophobic macroinitiator such that a block of units of the hydrophilic monomer grows from the site of functionalization of the hydrophobic macroinitiator. Suitable macroinitiators include a thermally or photochemically activatable radical initiator group. The initiator group is linked to the hydrophobic macroinitiator in a way that provides a covalent non-hydrolyzable bond between the terminal group of the hydrophobic macroinitiator and the first hydrophilic monomer forming the growing segment during the copolymerization for preparing the amphiphilic block copolymer.

It is also possible to change the monomer during the copolymerization such that, for example, first hydrophilic segments A are grown on a preformed hydrophobic segment B and then hydrophilic segments A′ are attached to the termini of the earlier prepared segments A. Similarly, a hydrophilic segment AA′ can be grown on a preformed hydrophobic segment B, by simultaneously using 2 or more hydrophilic monomers.

Accordingly, the amphiphilic block copolymer may consist in one embodiment of one hydrophilic segment A and one hydrophobic segment B (A-B-type, diblock), or of one hydrophobic segment B and two hydrophilic segments A attached to its termini (A-B-A-type, tri-block). In another embodiment, the amphiphilic block copolymer may consist of one hydrophilic segment AA′ made from 2 or more hydrophilic monomers and one hydrophobic segment B (AA′-B-type, diblock), or of one hydrophobic segment B and two hydrophilic segments AA′ attached to its termini (AA′-B-AA′, tri-block).

Additionally the amphiphilic block copolymers are substantially non-polymerizable. As used herein, substantially non-polymerizable means that when the amphiphilic block copolymers are polymerized with other polymerizable components, the amphiphilic block copolymers are incorporated into hydrogel formulations without significant covalent bonding to the hydrogel. The absence of significant covalent bonding means that while a minor degree of covalent bonding may be present, it is incidental to the retention of the amphiphilic block copolymer in the hydrogel matrix. Whatever incidental covalent bonding may be present, it would not by itself be sufficient to retain the amphiphilic block copolymer in the hydrogel matrix. Instead, the vastly predominating effect keeping the amphiphilic block copolymer associated with the hydrogel is entrapment. The amphiphilic block copolymer is “entrapped”, according to this specification, when it is physically retained within a hydrogel matrix. This is done via entanglement of the polymer chain of the amphiphilic block copolymer within the hydrogel polymer matrix. However, van der Waals forces, dipole-dipole interactions, electrostatic attraction and hydrogen bonding can also contribute to this entrapment to a lesser extent.

The length of one or more segments A or AA′ which are to copolymerized on the starting hydrophobic segment B can be easily controlled by controlling the amount of hydrophilic monomer which is added for the copolymerization. In this way the size of the segments and their ratio can be easily controlled. After polymerization of the hydrophilic monomers is complete, the resultant amphiphilic block copolymers have a weight average molecular weight sufficient such that said amphiphilic copolymers upon incorporation to silicone hydrogel formulations, improve the wettability of the cured silicone hydrogels.

Suitable polysiloxanes include blocks may be formed from silicone compounds with one or more reactive groups. Examples of such silicone compounds include linear polydimethylsiloxanes with terminal reactive groups. Reactive groups that may be useful include hydroxyl, carboxyl, amino, hydrosilyl, vinylsilyl, isocyanato, azo, acid halide, silanol and alkoxysilyl groups. The silicone groups may be positioned either in the primary chain or pendant to the primary chain. These silicone compounds may themselves be formed by any of a number of methods known to those skilled in the art, including condensation, ring-opening equilibration, or vinyl polymerization, from starting materials such as octamethylcyclotetrasiloxane; 1,3-bis-aminopropyltetramethyldisiloxane; 1,3-bis-hydroxypropyltetramethyldisiloxane; dichlorodimethylsilane, 1,1,3,3-tetramethyldisiloxane; 4,4′-azobis(4-cyanovaleric acid); toluenediisocyanate, isophoronediisocyanate; 1,3-bis-vinyltetramethyldisiloxane; 3-methacryloxypropyltris(trimethylsiloxy)silane; pentamethyldisiloxanyl methylmethacrylate; and methyldi(trimethylsiloxy)methacryloxymethyl silane; monomethacryloxypropyl terminated mono-n-butyl terminated polydimethylsiloxane; 3-[tris(trimethylsiloxy)silyl]propyl allyl carbamate; 3-[tris(trimethylsiloxy)wilyl]propyl vinyl carbamate; trimethylsilylethyl vinyl carbonate; trimethylsilylmethyl vinyl carbonate; and 2-propenoic acid, 2-methyl-2-hydroxy-3-[3-[1,3,3,3-tetramethyl-1-[trimethylsilyl)oxy]disilo-xanyl]propoxy]propyl ester, and combinations thereof.

One approach to improve the stability of polymeric micelles is to increase the hydrophobicity of the polymer. To do so, the molecular weight or the concentration of the polymer should be adjusted. However, as the molecular weight is increased, its biodegradability is decreased, and so the polymer is poorly excreted from the body and accumulates in organs causing toxic effects therein. U.S. Pat. No. 5,429,826 discloses a di- or multi-block copolymer comprising a hydrophilic polyalkylene glycol and a hydrophobic polylactic acid. Specifically, this patent describes a method of stabilizing polymeric micelles by micellizing a di- or multi-block copolymer wherein an acrylic acid derivative is bonded to a terminal group of the di- or multi-block copolymer, and then, in an aqueous solution, the polymer is crosslinked in order to form the micelles. The above method could accomplish stabilization of the polymeric micelle, but the crosslinked polymer is not degraded, and thus, cannot be applied for in vivo use. The above polymeric micelles can solubilize a large amount of poorly water-soluble drug in an aqueous solution with a neutral pH, but the drawback a that the drug is released within a short period of time. Also, in U.S. Pat. No. 6,458,373, a poorly water-soluble drug is solubilized into the form of an emulsion with α-tocopherol. According to this patent, to stabilize the emulsion, PEGylated vitamin E is used as a amphiphile molecule. PEGylated vitamin E has a similar structure to the amphiphilic block copolymer comprised of a hydrophilic block and a hydrophobic block, and the highly hydrophobic tocopherol increases the copolymer's affinity with a poorly water-soluble drug, and thus, it can solubilize the poorly water-soluble drug. However, polyethylene glycol used as the hydrophilic polymer has a limited molecular weight, and so PEGylated vitamin E alone can solubilize a hydrophobic drug such as paclitaxel only up to 2.5 mg/ml. At 2.5 mg/ml or more, unstable micelles are formed, and the drug crystals are likely to form precipitates.

Block copolymers having a variety of architectures, e.g. A-B, A-B-A and star-shaped block copolymers are known in the art. Among A-B type diblock copolymers, monomethoxy poly(ethylene glycol)-block-poly(D,L-lactide) (MPEG-b-PDLLA) (Yasugi, K.; Nagasaki, Y.; Kato, M.; Kataoka, K. 1999, J. Controlled Rel. 62, 89-100); monomethoxy poly(ethylene glycol)-block-poly(.epsilon.-caprolactone) (MPEG-b-PCL) (Shin, I. G.; Kim, S. Y.; Lee, Y. M., Cho, C. S.; Sung, Y. K. 1998, J. Controlled Rel. 51, 1-11) and monomethoxy poly(ethylene glycol)-block-poly(.beta. benzyl L-aspartate) (MPEG-b-PBLA) (Yokoyama, M.; Miyauchi, M.; Yamada, N.; Okano, T.; Sakurai, Y.; Kataoka, K.; Lnoue, S. 1990, J. Controlled Rel. 11, 269-278) have been extensively studied for micellar drug delivery. MPEG-b-PDLLA has been synthesized by ring opening polymerization of D,L-lactide initiated either with potassium monomethoxy poly(ethylene glyco)late at 25.degree. C. in tetrahydrofuran (THF) (Jeong, B.; Bae, Y. H.; Lee, D. S.; Kim, S. W. 1997, Nature 388, 860-862) or with MPEG at 110 to 150.degree. C. in the bulk (Kim, S. Y.; Shin, I. G.; Lee, Y. M. 1998, J. Controlled Rel. 56, 197-208). Similarly, MPEG-b-PCL has also been synthesized by ring opening polymerization of .epsilon.-caprolactone initiated with potassium MPEG alcoholate in THF at 25.degree. C. (Deng, X. M.; Zhu, Z. X.; Xiong, C. D.; Zhang, L. L. 1997, J. Polym. Sci. Polym. Chem. Ed. 35, 703-708) or with MPEG at 140 to 180.degree. C. in the bulk (Cerrai, P.; Tricoli, M.; Andruzzi, F.; Poci, M.; Pasi, M. 1989, Polymer 30, 338-343). MPEG-b-PBLA was synthesized by polymerization of N-carboxyanhydride of aspartic acid initiated with MPEG amine, in a solvent at 25.degree. C. (Yokoyama, M.; Lnoue, S.; Kataoka, K.; Yui, N.; Sakurai, Y. 1987, Makromol. Chem. Rapid Commun. 8, 431-435).

Among the different drug molecules that have been loaded in diblock copolymer micelles, are paclitaxel (Zhang, X.; Jackson, J. K.; Burt, H. M. 1996, Int. J. Pharm. 132, 195-206); testosterone (Allen, C.; Eisenberg, A.; Mrsic, J.; Maysinger, D. 2000, Drug Deliv. 7, 139-145); indomethacin (Kim, S. Y.,; Shin, I. G.; Lee, Y. M.; Cho, C. S.; Sung, Y. K. 1998, J. Controlled Rel. 51, 13-22); FK 506, L-685, 818 (Allen, C.; Yu, Y.; Maysinger, D.; Eisenberg, A. 1998, Bioconjug. Chem. 9, 564-572); dihydrotestosterone (Allen, C.; Han, J.; Yu, Y.; Maysinger, D.; Eisenberg, A. 2000, J. Controlled Rel. 63, 275-286); amphotericin B (Kwon, G. S.; Naito, M.; Yokoyama, M.; Okano, T.; Sakurai, Y.; Kataoka, Y. 1998, J. Controlled Rel. 51, 169-178); doxorubicin (Yu, B. G.; Okano, T.; Kataoka, K.; Kwon, G. 1998, J. Controlled Rel. 53, 131-136) and KRN (Yokoyama, M.; Satoh, A.; Sakurai, Y.; Okano, T.; Matsumara, Y.; Kakizoe, T.; Kataoka, K. 1998, J. Controlled Rel. 55, 219-229). In some cases, the incorporation of drugs into polymeric micelles has resulted in increased efficacy or decreased side-effects.

Among A-B-A type triblock copolymer compositions, poly(ethylene oxide)-block-poly(propylene oxide)-block-poly(ethylene oxide) based drug-loaded micelles have received extensive study (Kabanov, A. V. et al., 1989, FEBS Lett. 258, 343-345; Batrakova, E. V. et al 1996, Br. J. Cancer 74, 1545-1552; Batrakova, E. V.; Han, H. Y.; Alakhov, V. Y.; Miller, D. W.; Kabanov, A. V. 1998, Pharm. Res. 15 850-855; Rapoport, N. Y.; Marin, A.; Luo, Y.; Prestwich, G. D.; Muniruzzaman, M. J. 2002, Pharm. Sci. 91, 157-170; Rapport, N.Y., Herron, J. N.; Pitt, W. G.; Pitina, L. 1999, J. Controlled Rel. 58, 153-162; Cheng, H. Y.; Holl, W. W. 1990, J. Pharm. Sci. 79, 907-912). However, these polymers do not constitute a biodegradable embodiment. In an effort to develop such an embodiment, researchers have developed various biodegradable, amphiphilic A-B-A triblock copolymers.

U.S. Pat. No. 6,322,805 relates to biodegradable polymeric micelles capable of solubilizing a hydrophobic drug in a hydrophilic environment comprising an amphiphilic block copolymer having a hydrophilic poly(alkylene oxide) component and a biodegradable hydrophobic polymer selected from the group consisting of poly(lactic acid), poly(glycolic acid), poly(lactic-co-glycolic acid), poly(ε-caprolactone) and derivatives and mixtures thereof. The patent broadly teaches A-B-A type triblock copolymers which may contain poly(E -caprolactone) as one of their constituents, but fails to disclose the particular hydrophilic vinyl polymers comprising block copolymers set forth in the instant invention, nor a method by which such polymers could be successfully synthesized.

U.S. Pat. No. 6,201,065 is directed toward gel-forming macromers including at least four polymer blocks including at least two hydrophilic groups, one hydrophobic group and one crosslinkable group. The reference discloses the possible utilization of a plurality of polymerization techniques, among which is included attachment of a thiol to a reactant and subsequent covalent attachment to a macromer. The reference further teaches the formation of biodegradable links separating the cross-linking reactive groups. The reference fails to teach or suggest the particular type of block copolymers set forth in the instant invention, nor a method by which such polymers could be successfully synthesized.

Most of the reports cited above show that PEG has been the preferred choice of hydrophilic segment that imparts colloidal stability for block copolymer micelles. However, under certain conditions, PEG can promote the aggregation of nanoparticles after freeze-drying (De Jaghere, F.; Alleman, E.; Leroux, J.-C.; Stevels, W.; Feijen, J.; Doelker, E.; Gurny, R. 1999, Pharm. Res. 16, 859-866). Moreover, PEG chains are devoid of pendant sites that could be used to conjugate various functional groups for targeting or to induce pH and/or temperature sensitivity to the micelles. Hydrophilic polymers synthesized by polymerization or copolymerization of various vinyl monomers can provide such properties to the block copolymers. Examples of such block copolymers include poly(N-isopropylacrylamide)-block-poly(L-lactic acid) (Kim, I-S.; Jeong, Y-I.; Cho, C-S.; Kim, S-H. 2000, Int. J. Pharm. 211, 1-8); poly(N-isopropylacrylamide)-block-poly(butyl methacrylate) (Chung, J. E.; Yooyama, M.; Yamato, M.; Aoyagi, T.; Sakurai, Y., Okano, T. 1999, J. Controlled Rel. 62, 115-127); poly(N-isopropylacrylamide-co-methacrylic acid-co-octadecyl acrylate) (Taillefer, J.; Jones, M-C.; Brasseur, N.; Van Lier, J. E.; Leroux, J-C. 2000, J. Pharm. Sci. 89, 52-62). Moreover, structural variation of outer hydrophilic shells to produce micelles that can interact with many different biological environments is highly desirable.

Recently, Benhamed et al (2001) reported novel poly(N-vinylpyrrolidone)-block-poly(D,L-lactide) (PVP-b-PDLLA) micelles (Benhamed, A.; Ranger, M.; Leroux, J.-C. 2001, Pharm. Res. 18, 323-328). These micelles have potential advantage of the PVP shell being both lyoprotectant and cryoprotectant (Townsend, M.; Deluca, P. P. 1988, J. Parent. Sci. Technol. 37, 190-199; Doebbler, G. F. 1966, Cryobiology 3, 2-11). Also PVP, owing to its amphiphilic nature is capable of interacting with a variety of compounds (Garrett, Q.; Milthorpe, B. K. 1996, Invest. Ophthalmol. 37, 2594-2602; Alencar de Queiro, A. A.; Gallordo, A.; Romman, J. S. 2000, Biomaterials 21, 1631-1643). On the other hand, the group of Jeong et al (1999), (2000), reported the use of poly(2-ethyl-2-oxazoline) (PEtOz) as the shell-forming polymer in poly(2-ethyl-2-oxazoline)-block-poly(D,L-lactide) (PEtOz-b-PDLLA), poly(2-ethyl-2-oxazoline)-block-poly(.epsilon.-caprolactone) (PEtOz-b-PCL), and poly(2-ethyl-2-oxazoline)-block-poly(1,3 trimethylene carbonate) (PEtOz-b-PTMC). The hydrophilic shells in the above-described micelles form hydrogen-bonding complexes with poly(acrylic acid) that can dissociate above pH 3.9. (Lee, S. C.; Chang, Y.; Yoon, J.-S.; Kim, C.; Kwon, I. C.; Kim, Y-H; Jeong, S. Y. 1999, Macromolecules 32, 1847-1852; Kim, C.; Lee, S. C.; Shin, J. H.; Kwon, I. C.; Jeong, S. Y. 2000, Macromolecules 33, 7448-7452).

Poly(N-(2-hydroxypropyl)methacrylamide) (PHPMA) is another hydrophilic, non-immunogenic and biocompatible polymer. It has been demonstrated that anticancer drugs conjugated to PHPMA can exhibit stronger antitumor effects than the free drugs. Indeed, PK1 and PK2 are doxorubicin-conjugated PHPMA prodrugs that are now in clinical trials (Kopecek, J.; Kopecova, P.; Minko, T.; Lu, Z-R. 2000, Eur. J. Pharm. Biopharm. 50, 61-81). Free PHPMA has also been used as one of the components of poloxamer micelle-based chemotherapy liquid composition (Kabanov, A. V.; Alakhov, V. Y. 2000, U.S. Pat. No. 6,060,518). Moreover, block and graft copolymers of PHPMA with poly(L-lysine) and poly(trimethylaminoethylmethacrylate) have been described for gene delivery applications (Toncheva, V.; Wolfert, M. A.; Dash, P. R.; Oupicky, D.; Ulbrich, K.; Seymour, L. W.; Schacht, E. H. 1998, Biochim. Biophys. Acta. 1380, 354-368; Konack, C.; Mrkvickova, L.; Nazarova, O.; Ulbrich, K.; Seymour, L. W. 1998, Supramol. Sci. 5, 67-74).

The synthesis of block copolymers composed of hydrophobic biodegradable polymers and hydrophilic vinyl polymers has been previously reported by Hedrick et al (Hedrick, J. L.; Trollsas, M.; Hawker, C. J.; Atthoff, B.; Claesson, H.; Heise, A.; Miller, R. D.; Mecerreyes, D.; Jerome, R.; Dubois, Ph. 1998, Macromolecules 31, 8691-8705). However, in this study the authors used atom transfer radical polymerization (ATRP) to prepare the copolymers. Unfortunately, ATRP is not optimal for the polymerization of many vinyl monomers (e.g. HPMA, VP). The present inventors therefore decided to radically polymerize hydrophilic vinyl monomer in the presence of macromolecular biodegradable chain transfer-agent and obtain the block copolymers thereof. In the prior art, Sato et al (1987) synthesized a variety of A-B and A-B-A type block copolymers by free radical polymerization of vinyl monomers, such as vinyl acetate, methyl methacrylate, N,N-dimethylacrylamide and acrylic acid, in the presence of mono or dithiol-terminated PEG, poly(propylene glycol), poly(methyl methacrylate), poly(vinyl alcohol) and poly(styrene) as chain-transfer agents (Sato, T.; Yamauchi, J.; Okaya, T. 1987, U.S. Pat. No. 4,699,950). Inoue et al (1998) synthesized A-B type block copolymer micelles by radical polymerization of acrylic acid in the presence of thiol-terminated oligo(methyl methacrylate) as chain-transfer agent (Inoue, T.; Chen, G.; Nakame, K.; Hoffman, A. S. 1998, J. Controlled Rel. 51, 221-229). However, prior artisans failed to teach or suggest the use of macromolecular biodegradable chain-transfer agent.

3. Micelles

“Micelle” as used herein refers to a structure comprising an outer lipid monolayer. Micelles can be formed in an aqueous medium when the Critical Micelle Concentration (CMC) is exceeded. Small micelles in dilute solution at approximately the critical micelle concentration (CMC) are generally believed to be spherical. However, under other conditions, they may be in the shape of distorted spheres, disks, rods, lamellae, and the like. Micelles formed from relatively low molecular weight amphiphile molecules can have a high CMC so that the formed micelles dissociate rather rapidly upon dilution. If this is undesired, amphiphile molecules with large hydrophobic regions can be used. For example, lipids with a long fatty acid chain or two fatty acid chains, such as phospholipids and sphingolipids, or polymers, specifically block copolymers, can be used.

Polymeric micelles have been prepared that exhibit CMCs as low as 10⁻⁶ M (molar). Thus, they tend to be very stable while at the same time showing the same beneficial characteristics as amphiphile micelles. Any micelle-forming polymer presently known in the art or as such may become known in the future may be used in the disclosed compositions and methods. Examples of micelle-forming polymers include, without limitation, methoxy poly(ethylene glycol)-b-poly(ε-caprolactone), conjugates of poly(ethylene glycol) with phosphatidyl-ethanolamine, poly(ethylene glycol)-b-polyesters, poly(ethylene glycol)-b-poly(L-aminoacids), poly(N-vinylpyrrolidone)-bl-poly(orthoesters), poly(N-vinylpyrrolidone)-b-polyanhydrides and poly(N-vinylpyrrolidone)-b-poly(alkyl acrylates).

Micelles can be produced by processes conventional in the art. Examples of such are described in, for example, Liggins (Liggins, R. T. and Burt, H. M., “Polyether-polyester diblock copolymers for the preparation of paclitaxel loaded polymeric micelle formulations.” Adv. Drug Del. Rev. 54: 191-202, (2002)); Zhang, et al. (Zhang, X. et al., “Development of amphiphilic dibiock copolymers as micellar carriers of taxol.” Int. J. Pharm. 132: 195-206, (1996)); and Churchill (Churchill, J. R., and Hutchinson, F. G., “Biodegradable amphipathic copolymers.” U.S. Pat. No. 4,745,160, (1988)). In one such method, polyether-polyester block copolymers, which are amphipathic polymers having hydrophilic (polyether) and hydrophobic (polyester) segments, are used as micelle forming carriers.

Another type of micelle can be formed using, for example, AB-type block copolymers having both hydrophilic and hydrophobic segments, as described in, for example, Tuzar (Tuzar, Z. and Kratochvil, P., “Block and graft copolymer micelles in solution.”, Adv. Colloid Interface Sci. 6:201-232, (1976)); and Wilhelm, et al. (Wilhelm, M. et al., “Poly(styrene-ethylene oxide) block copolymer micelle formation in water: a fluorescence probe study.”, Macromolecules 24: 1033-1040 (1991)). These polymeric micelles are able to maintain satisfactory aqueous stability. These micelles, in the range of approximately <200 nm in size, are effective in reducing non-selective RES scavenging and show enhanced permeability and retention.

Further, U.S. Pat. No. 5,929,177 to Kataoka, et al. describes a polymeric molecule which is usable as, inter alia, a drug delivery carrier. The micelle is formed from a block copolymer having functional groups on both of its ends and which comprises hydrophilic/hydrophobic segments. The polymer functional groups on the ends of the block copolymer include amino, carboxyl and mercapto groups on the .alpha.-terminal and hydroxyl, carboxyl group, aldehyde group and vinyl group on the .omega.-terminal. The hydrophilic segment comprises polyethylene oxide, while the hydrophobic segment is derived from lactide, lactone or (meth)acrylic acid ester.

Further, for example, poly(D,L-lactide)-b-methoxypolyethylene glycol (MePEG:PDLLA) diblock copolymers can be made using MePEG 1900 and 5000. The reaction can be allowed to proceed for 3 hr at 160° C., using stannous octoate (0.25%) as a catalyst. However, a temperature as low as 130° C. can be used if the reaction is allowed to proceed for about 6 hr, or a temperature as high as 190° C. can be used if the reaction is carried out for only about 2 hr.

As another example, N-isopropylacrylamide (“IPAAm”) (Kohjin, Tokyo, Japan) and dimethylacrylamide (“DMAAm”) (Wako Pure Chemicals, Tokyo, Japan) can be used to make hydroxyl-terminated poly(IPAAm-co-DMAAm) in a radical polymerization process, using the method of Kohori, F. et al. (1998). (Kohori, F. et al., “Preparation and characterization of thermally Responsive block copolymer micelles comprising poly(N-isopropylacrylamide-b-D,L-lactide).” J. Control. Rel. 55: 87-98, (1998)). The obtained copolymer can be dissolved in cold water and filtered through two ultrafiltration membranes with a 10,000 and 20,000 molecular weight cut-off. The polymer solution is first filtered through a 20,000 molecular weight cut-off membrane. Then the filtrate was filtered again through a 10,000 molecular weight cut-off membrane. Three molecular weight fractions can be obtained as a result, a low molecular weight, a middle molecular weight, and a high molecular weight fraction. A block copolymer can then be synthesized by a ring opening polymerization of D,L-lactide from the terminal hydroxyl group of the poly(IPAAm-co-DMAAm) of the middle molecular weight fraction. The resulting poly(IPAAm-co-DMAAm)-b-poly(D,L-lactide) copolymer can be purified as described in Kohori, F. et al. (1999). (Kohori, F. et al., “Control of adriamycin cytotoxic activity using thermally responsive polymeric micelles composed of poly(N-isopropylacrylamide-co-N,N-dimethylacrylamide)-b-poly(D,L-lacide).-”, Colloids Surfaces B: Biointerfaces 16: 195-205, (1999)).

Examples of block copolymers from which micelles can be prepared which can be used to coat a support surface are found in U.S. Pat. No. 5,925,720, to Kataoka, et al., U.S. Pat. No. 5,412,072 to Sakarai, et al., U.S. Pat. No. 5,410,016 to Kataoka, et al., U.S. Pat. No. 5,929,177 to Kataoka, et al., U.S. Pat. No. 5,693,751 to Sakurai, et al., U.S. Pat. No. 5,449,513 to Yokoyama, et al., WO 96/32434, WO 96/33233 and WO 97/0623, the contents of all of which are incorporated by reference. Modifications thereof which are prepared by introducing thereon a suitable functional group (including an ethyleneically unsaturated polymerizable group) are also examples of block copolymers from which micelles of the present invention are preferably prepared. Preferable block copolymers are those disclosed in the above-mentioned patents and or international patent publications. If the block copolymer has a sugar residue on one end of the hydrophilic polymer segment, as in the block copolymer of WO 96/32434, the sugar residue should preferably be subjected to Malaprade oxidation so that a corresponding aldehyde group may be formed.

4. Liposomes

“Liposome” as the term is used herein refers to a structure comprising an outer lipid bi- or multi-layer membrane surrounding an internal aqueous space. Liposomes can be used to package any biologically active agent for delivery to cells.

Materials and procedures for forming liposomes are well-known to those skilled in the art. Upon dispersion in an appropriate medium, a wide variety of phospholipids swell, hydrate and form multilamellar concentric bilayer vesicles with layers of aqueous media separating the lipid bilayers. These systems are referred to as multilamellar liposomes or multilamellar lipid vesicles (“MLVs”) and have diameters within the range of 10 nm to 100 μm. These MLVs were first described by Bangham, et al., J Mol. Biol. 13:238-252 (1965). In general, lipids or lipophilic substances are dissolved in an organic solvent. When the solvent is removed, such as under vacuum by rotary evaporation, the lipid residue forms a film on the wall of the container. An aqueous solution that typically contains electrolytes or hydrophilic biologically active materials is then added to the film. Large MLVs are produced upon agitation. When smaller MLVs are desired, the larger vesicles are subjected to sonication, sequential filtration through filters with decreasing pore size or reduced by other forms of mechanical shearing. There are also techniques by which MLVs can be reduced both in size and in number of lamellae, for example, by pressurized extrusion (Barenholz, et al., FEBS Lett. 99:210-214 (1979)).

Liposomes can also take the form of unilamnellar vesicles, which are prepared by more extensive sonication of MLVs, and consist of a single spherical lipid bilayer surrounding an aqueous solution. Unilamellar vesicles (“ULVs”) can be small, having diameters within the range of 20 to 200 nm, while larger ULVs can have diameters within the range of 200 nm to 2 μm. There are several well-known techniques for making unilamellar vesicles. In Papahadjopoulos, et al., Biochim et Biophys Acta 135:624-238 (1968), sonication of an aqueous dispersion of phospholipids produces small ULVs having a lipid bilayer surrounding an aqueous solution. Schneider, U.S. Pat. No. 4,089,801 describes the formation of liposome precursors by ultrasonication, followed by the addition of an aqueous medium containing amphiphilic compounds and centrifugation to form a biomolecular lipid layer system.

Small ULVs can also be prepared by the ethanol injection technique described by Batzri, et al., Biochim et Biophys Acta 298:1015-1019 (1973) and the ether injection technique of Deamer, et al., Biochim et Biophys Acta 443:629-634 (1976). These methods involve the rapid injection of an organic solution of lipids into a buffer solution, which results in the rapid formation of unilamellar liposomes. Another technique for making ULVs is taught by Weder, et al. in “Liposome Technology”, ed. G. Gregoriadis, CRC Press Inc., Boca Raton, Fla., Vol. I, Chapter 7, pg. 79-107 (1984). This detergent removal method involves solubilizing the lipids and additives with detergents by agitation or sonication to produce the desired vesicles.

Papahadjopoulos, et al., U.S. Pat. No. 4,235,871, describes the preparation of large ULVs by a reverse phase evaporation technique that involves the formation of a water-in-oil emulsion of lipids in an organic solvent and the drug to be encapsulated in an aqueous buffer solution. The organic solvent is removed under pressure to yield a mixture which, upon agitation or dispersion in an aqueous media, is converted to large ULVs. Suzuki et al., U.S. Pat. No. 4,016,100, describes another method of encapsulating agents in unilamellar vesicles by freezing/thawing an aqueous phospholipid dispersion of the agent and lipids.

In addition to the MLVs and ULVs, liposomes can also be multivesicular. Described in Kim, et al., Biochim et Biophys Acta 728:339-348 (1983), these multivesicular liposomes are spherical and contain internal granular structures. The outer membrane is a lipid bilayer and the internal region contains small compartments separated by bilayer septum. Still yet another type of liposomes are oligolamellar vesicles (“OLVs”), which have a large center compartment surrounded by several peripheral lipid layers. These vesicles, having a diameter of 2-15 μm, are described in Callo, et al., Cryobiology 22(3):251-267 (1985).

Mezei, et al., U.S. Pat. Nos. 4,485,054 and 4,761,288 also describe methods of preparing lipid vesicles. More recently, Hsu, U.S. Pat. No. 5,653,996 describes a method of preparing liposomes utilizing aerosolization and Yiournas, et al., U.S. Pat. No. 5,013,497 describes a method for preparing liposomes utilizing a high velocity-shear mixing chamber. Methods are also described that use specific starting materials to produce ULVs (Wallach, et al., U.S. Pat. No. 4,853,228) or OLVs (Wallach, U.S. Pat. Nos. 5,474,848 and 5,628,936).

A comprehensive review of all the aforementioned lipid vesicles and methods for their preparation are described in “Liposome Technology”, ed. G. Gregoriadis, CRC Press Inc., Boca Raton, Fla., Vol. I, II & III (1984). This and the aforementioned references describing various lipid vesicles suitable for use in the invention are incorporated herein by reference.

i. Preparation of Liposomes

A variety of methods are available for preparing liposomes as described in, e.g., Szoka, et al., Ann. Rev. Biophys. Bioeng., 9:467 (1980), U.S. Pat. Nos. 4,186,183, 4,217,344, 4,235,871, 4,261,975, 4,485,054, 4,501,728, 4,774,085, 4,837,028, 4,946,787, PCT Publication No. WO 91/17424, Deamer and Bangham, Biochim. Biophys. Acta, 443:629-634 (1976); Fraley, et al., Proc. Natl. Acad. Sci. USA, 76:3348-3352 (1979); Hope, et al., Biochim. Biophys. Acta, 812:55-65 (1985); Mayer, et al., Biochim. Biophys. Acta, 858:161-168 (1986); Williams, et al., Proc. Natl. Acad. Sci., 85:242-246 (1988), the text Liposomes, Marc J. Ostro, ed., Marcel Dekker, Inc., New York, 1983, Chapter 1, and Hope, et al., Chem. Phys. Lip., 40:89 (1986), all of which are incorporated herein by reference. Suitable methods include, but are not limited to, sonication, extrusion, high pressure/homogenization, microfluidization, detergent dialysis, calcium-induced fusion of small liposome vesicles, and ether-infusion methods, all of which are well known in the art.

Liposomes can be prepared by, for example, dissolving the amphiphile molecule in an organic solvent, allowing formation of a thin film on a surface, hydrating the film and filtering the resultant solution to obtain liposomes. This method is illustrated in FIG. 16 (using a peptide amphiphile as an example of the amphiphile molecule).

Alternative methods of preparing liposomes are also available. For instance, a method involving detergent dialysis based self-assembly of lipid particles is disclosed and claimed in U.S. Pat. No. 5,976,567 issued to Wheeler, et al., which avoids the time-consuming and difficult to-scale drying and reconstitution steps. Further methods of preparing liposomes using continuous flow hydration are under development and can often provide the most effective large scale manufacturing process.

One method produces multilamellar vesicles of heterogeneous sizes. In this method, the vesicle-forming lipids are dissolved in a suitable organic solvent or solvent system and dried under vacuum or an inert gas to form a thin lipid film. If desired, the film may be redissolved in a suitable solvent, such as tertiary butanol, and then lyophilized to form a more homogeneous lipid mixture which is in a more easily hydrated powder-like form. This film is covered with an aqueous buffered solution and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents, such as deoxycholate.

Unilamellar vesicles can be prepared by sonication or extrusion. Sonication is generally performed with a tip sonifier, such as a Branson tip sonifier, in an ice bath. Typically, the suspension is subjected to severed sonication cycles. Extrusion may be carried out by biomembrane extruders, such as the Lipex Biomembrane Extruder. Defined pore size in the extrusion filters may generate unilamellar liposomal vesicles of specific sizes. The liposomes may also be formed by extrusion through an asymmetric ceramic filter, such as a Ceraflow Microfilter, commercially available from the Norton Company, Worcester Mass. Unilamellar vesicles can also be made by dissolving phospholipids in ethanol and then injecting the lipids into a buffer, causing the lipids to spontaneously form unilamellar vesicles. Also, phospholipids can be solubilized into a detergent, e.g., cholates, Triton X, or n-alkylglucosides. Following the addition of the drug to the solubilized lipid-detergent micelles, the detergent is removed by any of a number of possible methods including dialysis, gel filtration, affinity chromatography, centrifugation, and ultrafiltration.

Following liposome preparation, the liposomes which have not been sized during formation may be sized to achieve a desired size range and relatively narrow distribution of liposome sizes. A size range of about 0.2-0.4 microns allows the liposome suspension to be sterilized by filtration through a conventional filter. The filter sterilization method can be carried out on a high through-put basis if the liposomes have been sized down to about 0.2-0.4 microns.

Several techniques are available for sizing liposomes to a desired size. One sizing method is described in U.S. Pat. No. 4,737,323, incorporated herein by reference. Sonicating a liposome suspension either by bath or probe sonication produces a progressive size reduction down to small unilamellar vesicles less than about 0.05 microns in size. Homogenization is another method that relies on shearing energy to fragment large liposomes into smaller ones. In a typical homogenization procedure, multilamellar vesicles are recirculated through a standard emulsion homogenizer until selected liposome sizes, typically between about 0.1 and 0.5 microns, are observed. The size of the liposomal vesicles may be determined by quasi-electric light scattering (QELS) as described in Bloomfield, Ann. Rev. Biophys. Bioeng., 10:421-450 (1981), incorporated herein by reference. Average liposome diameter may be reduced by sonication of formed liposomes. Intermittent sonication cycles may be alternated with QELS assessment to guide efficient liposome synthesis.

Extrusion of liposome through a small-pore polycarbonate membrane or an asymmetric ceramic membrane is also an effective method for reducing liposome sizes to a relatively well-defined size distribution. Typically, the suspension is cycled through the membrane one or more times until the desired liposome size distribution is achieved. The liposomes may be extruded through successively smaller-pore membranes, to achieve gradual reduction in liposome size

Liposomes prepared according to these methods can be stored for substantial periods of time prior to drug loading and administration to a patient. For example, liposomes can be dehydrated, stored, and subsequently rehydrated, loaded with one or more vinca alkaloids, and administered. Dehydration can be accomplished, e.g., using standard freeze-drying apparatus, i.e., they are dehydrated under low pressure conditions. Also, the liposomes can be frozen, e.g., in liquid nitrogen, prior to dehydration. Sugars can be added to the liposomal environment, e.g., to the buffer containing the liposomes, prior to dehydration, thereby promoting the integrity of the liposome during dehydration. See, e.g., U.S. Pat. No. 5,077,056 or 5,736,155.

### B. Head Groups

The compositions and/or the amphiphile molecules disclosed herein can further comprise one or more head groups. Head groups can be, for example, targeting head groups and functional head groups. Targeting head groups can be, for example, clot-binding head groups. Functional head groups can be, for example, detection head groups and treatment head groups. Head groups can also combine two or more of the properties of the different types of head groups. For example, a treatment head group can also be detectable and thus also be considered a detection head group. In some forms, the head groups can be independently selected from the group consisting of clot-binding head group, an anti-angiogenic agent, a pro-angiogenic agent, a cancer chemotherapeutic agent, a cytotoxic agent, an anti-inflammatory agent, an anti-arthritic agent, a polypeptide, a nucleic acid molecule, a small molecule, a fluorophore, fluorescein, rhodamine, a radionuclide, indium-111, technetium-99, carbon-11, and carbon-13. At least one of the head groups can be a treatment head group. Examples of treatment head groups are paclitaxel and taxol. At least one of the head groups can be a detection head group.

As used herein, the term “head group” is used broadly to mean a physical, chemical, or biological material that generally imparts a biologically useful function to a linked or conjugated molecule. The description of treatment and detection head groups which follows is intended to apply to any of head groups, amphiphile molecules, or clot-binding head groups. Thus, for example, head groups can be conjugated to, coupled to, or can be part of the disclosed amphiphile molecules, clot-binding head groups, or conjugates of amphiphile molecules and clot-binding head groups.

A head group can be any natural or nonnatural material including, without limitation, a biological material, such as a cell, phage or other virus; an organic chemical such as a small molecule; a radionuclide; a nucleic acid molecule or oligonucleotide; a polypeptide; or a peptide. Useful head groups include, but are not limited to, clot-binding head groups and treatment head groups such as cancer chemotherapeutic agents, cytotoxic agents, pro-apoptotic agents, and anti-angiogenic agents; detectable labels and imaging agents; and tags or other insoluble supports. Useful head groups further include, without limitation, phage and other viruses, cells, liposomes, polymeric matrices, non-polymeric matrices or particles such as gold particles, microdevices and nanodevices, and nano-scale semiconductor materials. These and other head groups known in the art can be components of a composition.

1. Clot-Binding Head Groups

The clot-binding head group can be any compound with the ability to interact with clots and/or components of clots such as clotted plasma proteins. The composition can comprise a sufficient number and composition of clot-binding head groups such that the composition causes clotting the accumulation of the composition at sites of clotting, at the sites of plaques, and at the site of injury. In one example, sufficiency of the number and composition of clot-binding head groups can be determined by assessing the accumulation of the composition at sites of clotting, at the sites of plaques, and/or at the site of injury in a non-human animal.

The clot-binding head groups can each be independently selected from, for example, an amino acid segment comprising the amino acid sequence REK, a fibrin-binding peptide, a peptide that binds clots and not fibrin (such as CGLIIQKNEC (CLT1, SEQ ID NO: 2) and CNAGESSKNC (CLT2, SEQ ID NO: 3)), a clot-binding antibody, and a clot-binding small organic molecule. The clot-binding head groups can each independently comprise an amino acid segment comprising the amino acid sequence REK. Such peptides are also described in U.S. Patent Application Publication No. 2008/0305101, which is hereby incorporated by reference for its description of such peptides. Peptides comprising amino acid sequences CAR or CRK are also described in U.S. Patent Application Publication No. 2009/0036349, which is hereby incorporated by reference for its description of such peptides.

The composition can comprise any number of clot-binding head groups. By way of example, the composition can comprise at least 1, 5, 10, 15, 20, 25, 50, 75, 100, 125, 150, 175, 200, 225, 250, 275, 300, 325, 350, 375, 400, 425, 450, 475, 500, 525, 550, 575, 600, 625, 650, 675, 700, 625, 750, 775, 800, 825, 850, 875, 900, 925, 950, 975, 1000, 1100, 1200, 1300, 1400, 1500, 1600, 1700, 1800, 1900, 2000, 2250, 2500, 2750, 3000, 3500, 4000, 4500, 5000, 5500, 6000, 6500, 7000, 7500, 8000, 8500, 9000, 9500, 10,000, 15,000, 20,000, 25,000, 30,000, 35,000, 40,000, 45,000, 50,000, 75,000, or 100,000, or more clot-binding head groups. The composition can also comprise any number in between those numbers listed above.

The term “homing molecule” as used herein, means any molecule that selectively homes in vivo to specified target sites or tissues in preference to normal tissue. Similarly, the term “homing peptide” or “homing peptidomimetic” means a peptide that selectively homes in vivo to specified target sites or tissues in preference to normal tissue. It is understood that a homing molecule that selectively homes in vivo to, for example, tumors can home to all tumors or can exhibit preferential homing to one or a subset of tumor types.

By “selectively homes” is meant that, in vivo, the homing molecule binds preferentially to the target as compared to non-target. For example, the homing molecule can bind preferentially to clotted plasma of one or more tumors, wound tissue, or blood clots, as compared to non-tumoral tissue or non-wound tissue. Such a homing molecule can selectively home, for example, to tumors. Selective homing to, for example, tumors generally is characterized by at least a two-fold greater localization within tumors (or other target), as compared to several tissue types of non-tumor tissue. A homing molecule can be characterized by 5-fold, 10-fold, 20-fold or more preferential localization to tumors (or other target) as compared to several or many tissue types of non-tumoral tissue, or as compared to-most or all non-tumoral tissue. Thus, it is understood that, in some cases, a homing molecule homes, in part, to one or more normal organs in addition to homing to the target tissue. Selective homing can also be referred to as targeting.

The disclosed clot-binding head groups can include modified forms of clot-binding head groups. The clot-binding head groups can have any useful modification. For example, some modifications can stabilize the clot-binging compound. For example, the disclosed clot-binding head groups include methylated clot-binding head groups. Methylated clot-binding head groups are particularly useful when the clot-binding head group includes a protein, peptide or amino acid segment. For example, a clot-binding head group can be a modified clot-binding head group, where, for example, the modified clot-binding head group includes a modified amino acid segment or amino acid sequence. For example, a modified clot-binding head group can be a methylated clot-binding head group, where, for example, the methylated clot-binding head group includes a methylated amino acid segment or amino acid sequence. Other modifications can be used, either alone or in combination. Where the clot-binding head group is, or includes, a protein, peptide, amino acid segment and/or amino acid sequences, the modification can be to the protein, peptide, amino acid segment, amino acid sequences and/or any amino acids in the protein, peptide, amino acid segment and/or amino acid sequences. Amino acid and peptide modifications are known to those of skill in the art, some of which are described below and elsewhere herein. Methylation is a particularly useful modification for the disclosed clot-binding head groups.

It has been discovered that by using modified forms of clot-binding head groups the effectiveness of the accumulation and/or delivery of the composition at sites of clotting, at the sites of plaques, and at the site of injury. The composition can comprise a sufficient number and composition of clot-binding head groups such that the composition causes clotting the accumulation of the composition at sites of clotting, at the sites of plaques, and at the site of injury. In one example, sufficiency of the number and composition of clot-binding head groups can be determined by assessing the accumulation of the composition at sites of clotting, at the sites of plaques, and/or at the site of injury in a non-human animal.

A plurality of modified and/or unmodified clot-binding head groups can each be independently selected from, for example, an amino acid segment comprising a modified or unmodified form of the amino acid sequence REK, an amino acid segment comprising a modified or unmodified form of the amino acid sequence CAR (such as CARSKNKDC (SEQ ID NO:6)), an amino acid segment comprising a modified or unmodified form of the amino acid sequence CRK (such as CRKDKC (SEQ ID NO:5)), a modified or unmodified form of a fibrin-binding peptide, a modified or unmodified form of a peptide that binds clots and not fibrin (such as CGLIIQKNEC (CLT1, SEQ ID NO: 2) and CNAGESSKNC (CLT2, SEQ ID NO: 3)), a modified or unmodified form of a clot-binding antibody, and a modified or unmodified form of a clot-binding small organic molecule. A plurality of the clot-binding head groups can each independently comprise an amino acid segment comprising a modified or unmodified form of the amino acid sequence REK. Such peptides are also described in U.S. Patent Application Publication No. 2008/0305101, which is hereby incorporated by reference for its description of such peptides. Peptides comprising amino acid sequences CAR or CRK are also described in U.S. Patent Application Publication No. 2009/0036349, which is hereby incorporated by reference for its description of such peptides.

The composition can comprise any number of modified and/or unmodified clot-binding head groups. By way of example, the composition can comprise at least 1, 5, 10, 15, 20, 25, 50, 75, 100, 125, 150, 175, 200, 225, 250, 275, 300, 325, 350, 375, 400, 425, 450, 475, 500, 525, 550, 575, 600, 625, 650, 675, 700, 625, 750, 775, 800, 825, 850, 875, 900, 925, 950, 975, 1000, 1100, 1200, 1300, 1400, 1500, 1600, 1700, 1800, 1900, 2000, 2250, 2500, 2750, 3000, 3500, 4000, 4500, 5000, 5500, 6000, 6500, 7000, 7500, 8000, 8500, 9000, 9500, 10,000, 15,000, 20,000, 25,000, 30,000, 35,000, 40,000, 45,000, 50,000, 75,000, or 100,000, or more modified and/or unmodified clot-binding head groups. The composition can also comprise any number in between those numbers listed above.

As used herein, a “methylated derivative” of a protein, peptide, amino acid segment, amino acid sequence, etc. refers to a form of the protein, peptide, amino acid segment, amino acid sequence, etc. that is methylated. Unless the context indicates otherwise, reference to a methylated derivative of a protein, peptide, amino acid segment, amino acid sequence, etc. does no include any modification to the base protein, peptide, amino acid segment, amino acid sequence, etc. other than methylation. Methylated derivatives can also have other modifications, but such modifications generally will be noted. For example, conservative variants of an amino acid sequence would include conservative amino acid substitutions of the based amino acid sequence. Thus, reference to, for example, a “methylated derivative” of a specific amino acid sequence “and conservative variants thereof” would include methylated forms of the specific amino acid sequence and methylated forms of the conservative variants of the specific amino acid sequence, but not any other modifications of derivations. As another example, reference to a methylated derivative of an amino acid segment that includes amino acid substitutions would include methylated forms of the amino acid sequence of the amino acid segment and methylated forms of the amino acid sequence of the amino acid segment include amino acid substitutions.

The clot-binding head groups and other peptides and proteins can have different or additional modifications as described elsewhere herein.

i. Peptides

In one example, the clot-binding head group can be a peptide or peptidomimetic. The disclosed peptides can be in isolated form. As used herein in reference to the disclosed peptides, the term “isolated” means a peptide that is in a form that is relatively free from material such as contaminating polypeptides, lipids, nucleic acids and other cellular material that normally is associated with the peptide in a cell or that is associated with the peptide in a library or in a crude preparation.

The disclosed peptides can have any suitable length. The disclosed peptides can have, for example, a relatively short length of less than six, seven, eight, nine, ten, 12, 15, 20, 25, 30, 35 or 40 residues. The disclosed peptides also can be useful in the context of a significantly longer sequence. Thus, the peptides can have, for example, a length of up to 50, 100, 150, 200, 250, 300, 400, 500, 1000 or 2000 residues. In particular embodiments, a peptide can have a length of at least 10, 20, 30, 40, 50, 60, 70, 80, 90, 100 or 200 residues. In further embodiments, a peptide can have a length of 5 to 200 residues, 5 to 100 residues, 5 to 90 residues, 5 to 80 residues, 5 to 70 residues, 5 to 60 residues, 5 to 50 residues, 5 to 40 residues, 5 to 30 residues, 5 to 20 residues, 5 to 15 residues, 5 to 10 residues, 10 to 200 residues, 10 to 100 residues, 10 to 90 residues, 10 to 80 residues, 10 to 70 residues, 10 to 60 residues, 10 to 50 residues, 10 to 40 residues, 10 to 30 residues, 10 to 20 residues, 20 to 200 residues, 20 to 100 residues, 20 to 90 residues, 20 to 80 residues, 20 to 70 residues, 20 to 60 residues, 20 to 50 residues, 20 to 40 residues or 20 to 30 residues. As used herein, the term “residue” refers to an amino acid or amino acid analog.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

The peptide can be circular (cyclic) or can contain a loop. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference). It is understood that, although many peptides, homing motifs and sequences, and targeting motifs and sequences are shown with cysteine residues at one or both ends, such cysteine residues are generally not required for homing function. Generally, such cysteines are present due to the methods by which the homing and targeting sequences were identified. Thus, any of the known or disclosed peptides, homing motifs and sequences, and targeting motifs and sequences that have one or two terminal cysteines can be used without such cysteines. Such forms of known or disclosed peptides, homing motifs and sequences, and targeting motifs and sequences are specifically contemplated herein. Such terminal cysteines can be used to, for example, circularize peptides, such as those disclosed herein. For these reasons, it is also understood that cysteine residues can be added to the ends of any of the disclosed peptides.

Peptides can have a variety of modifications. Modifications can be used to change or improve the properties of the peptides. For example, the disclosed peptides can be N-methylated, O-methylated, S-methylated, C-methylated, or a combination at one or more amino acids.

The amino and/or carboxy termini of the disclosed peptides can be modified. Amino terminus modifications include methylation (e.g., —NHCH₃ or —N(CH₃)₂), acetylation (e.g., with acetic acid or a halogenated derivative thereof such as α-chloroacetic acid, α-bromoacetic acid, or .alpha.-iodoacetic acid), adding a benzyloxycarbonyl (Cbz) group, or blocking the amino terminus with any blocking group containing a carboxylate functionality defined by RCOO— or sulfonyl functionality defined by R—SO₂—, where R is selected from the group consisting of alkyl, aryl, heteroaryl, alkyl aryl, and the like, and similar groups. One can also incorporate a desamino acid at the N-terminus (so that there is no N-terminal amino group) to decrease susceptibility to proteases or to restrict the conformation of the peptide compound. In preferred embodiments, the N-terminus is acetylated with acetic acid or acetic anhydride.

Carboxy terminus modifications include replacing the free acid with a carboxamide group or forming a cyclic lactam at the carboxy terminus to introduce structural constraints. One can also cyclize the disclosed peptides, or incorporate a desamino or descarboxy residue at the termini of the peptide, so that there is no terminal amino or carboxyl group, to decrease susceptibility to proteases or to restrict the conformation of the peptide. C-terminal functional groups of the disclosed peptides include amide, amide lower alkyl, amide di(lower alkyl), lower alkoxy, hydroxy, and carboxy, and the lower ester derivatives thereof, and the pharmaceutically acceptable salts thereof.

One can replace the naturally occurring side chains of the genetically encoded amino acids (or the stereoisomeric D amino acids) with other side chains, for instance with groups such as alkyl, lower (C₁₋₆) alkyl, cyclic 4-, 5-, 6-, to 7-membered alkyl, amide, amide lower alkyl amide di(lower alkyl), lower alkoxy, hydroxy, carboxy and the lower ester derivatives thereof, and with 4-, 5-, 6-, to 7-membered heterocyclic. In particular, proline analogues in which the ring size of the proline residue is changed from 5 members to 4, 6, or 7 members can be employed. Cyclic groups can be saturated or unsaturated, and if unsaturated, can be aromatic or non-aromatic. Heterocyclic groups preferably contain one or more nitrogen, oxygen, and/or sulfur heteroatoms. Examples of such groups include the furazanyl, furyl, imidazolidinyl, imidazolyl, imidazolinyl, isothiazolyl, isoxazolyl, morpholinyl (e.g. morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and triazolyl. These heterocyclic groups can be substituted or unsubstituted. Where a group is substituted, the substituent can be alkyl, alkoxy, halogen, oxygen, or substituted or unsubstituted phenyl.

One can also readily modify peptides by phosphorylation, and other methods [e.g., as described in Hruby, et al. (1990) Biochem J. 268:249-262].

The disclosed peptides also serve as structural models for non-peptidic compounds with similar biological activity. Those of skill in the art recognize that a variety of techniques are available for constructing compounds with the same or similar desired biological activity as the lead peptide compound, but with more favorable activity than the lead with respect to solubility, stability, and susceptibility to hydrolysis and proteolysis [See, Morgan and Gainor (1989) Ann. Rep. Med. Chem. 24:243-252]. These techniques include, but are not limited to, replacing the peptide backbone with a backbone composed of phosphonates, amidates, carbamates, sulfonamides, secondary amines, and N-methylamino acids.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO-(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂); Spatola et al. Life Sci 38:1243-1249 (1986) (—CHH₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as β-alanine, γ-aminobutyric acid, and the like.

Also disclosed are bifunctional peptides, which contain the clot-binding peptide fused to a second peptide having a separate function. Such bifunctional peptides have at least two functions conferred by different portions of the full-length molecule and can, for example, display anti-angiogenic activity or pro-apoptotic activity in addition to the ability to enhance clotting.

Also disclosed are isolated multivalent peptides that include at least two subsequences each independently containing a peptide (for example, the amino acid sequence SEQ ID NO: 1, or a conservative variant or peptidomimetic thereof). The multivalent peptide can have, for example, at least three, at least five or at least ten of such subsequences each independently containing a peptide. In particular embodiments, the multivalent peptide can have two, three, four, five, six, seven, eight, nine, ten, fifteen or twenty identical or non-identical subsequences. This is in addition to the multiple clot-binding head groups that can comprise the composition. In a further embodiment, the multivalent peptide can contain identical subsequences, such as repeats of SEQ ID NO: 1. In a further embodiment, the multivalent peptide contains contiguous identical or non-identical subsequences, which are not separated by any intervening amino acids.

As used herein, the term “peptide” is used broadly to mean peptides, proteins, fragments of proteins and the like. The term “peptidomimetic,” as used herein, means a peptide-like molecule that has the activity of the peptide upon which it is structurally based. Such peptidomimetics include chemically modified peptides, peptide-like molecules containing non-naturally occurring amino acids, and peptoids and have an activity such as selective interaction with a target of the peptide upon which the peptidomimetic is derived (see, for example, Goodman and Ro, Peptidomimetics for Drug Design, in “Burger's Medicinal Chemistry and Drug Discovery” Vol. 1 (ed. M. E. Wolff; John Wiley & Sons 1995), pages 803-861).

A variety of peptidomimetics are known in the art including, for example, peptide-like molecules which contain a constrained amino acid, a non-peptide component that mimics peptide secondary structure, or an amide bond isostere. A peptidomimetic that contains a constrained, non-naturally occurring amino acid can include, for example, an α-methylated amino acid; α,α.-dialkylglycine or α-aminocycloalkane carboxylic acid; an N^{α}—C^{α} cyclized amino acid; an N^{α}.-methylated amino acid; a β- or γ-amino cycloalkane carboxylic acid; an α,β-unsaturated amino acid; a β,β-dimethyl or β-methyl amino acid; a β-substituted-2,3-methano amino acid; an N—C^{ε} or C^{α}—C^{Δ} cyclized amino acid; a substituted proline or another amino acid mimetic. A peptidomimetic which mimics peptide secondary structure can contain, for example, a non-peptidic β-turn mimic; γ-turn mimic; mimic of β-sheet structure; or mimic of helical structure, each of which is well known in the art. A peptidomimetic also can be a peptide-like molecule which contains, for example, an amide bond isostere such as a retro-inverso modification; reduced amide bond; methylenethioether or methylene-sulfoxide bond; methylene ether bond; ethylene bond; thioamide bond; trans-olefin or fluoroolefin bond; 1,5-disubstituted tetrazole ring; ketomethylene or fluoroketomethylene bond or another amide isostere. One skilled in the art understands that these and other peptidomimetics are encompassed within the meaning of the term “peptidomimetic” as used herein.

Methods for identifying a peptidomimetic are well known in the art and include, for example, the screening of databases that contain libraries of potential peptidomimetics. As an example, the Cambridge Structural Database contains a collection of greater than 300,000 compounds that have known crystal structures (Allen et al., Acta Crystalloqr. Section B, 35:2331 (1979)). This structural depository is continually updated as new crystal structures are determined and can be screened for compounds having suitable shapes, for example, the same shape as a disclosed peptide, as well as potential geometrical and chemical complementarity to a target molecule. Where no crystal structure of a peptide or a target molecule that binds the peptide is available, a structure can be generated using, for example, the program CONCORD (Rusinko et al., J. Chem. Inf. Comput. Sci. 29:251 (1989)). Another database, the Available Chemicals Directory (Molecular Design Limited, Information Systems; San Leandro Calif.), contains about 100,000 compounds that are commercially available and also can be searched to identify potential peptidomimetics of a peptide, for example, with activity in selectively interacting with cancerous cells.

a. Homing Peptides

There are several examples in the art of peptides that home to clotted plasma protein. Examples include REK, peptides comprising REK, CREKA (SEQ ID NO: 1), and peptides comprising CREKA (SEQ ID NO: 1). The amino acid segments can also be independently selected from amino acid segments comprising the amino acid sequence CREKA (SEQ ID NO: 1) or a conservative variant thereof, amino acid segments comprising the amino acid sequence CREKA (SEQ ID NO:1), amino acid segments consisting of the amino acid sequence CREKA (SEQ ID NO:1), and amino acid segments consisting of the amino acid sequence REK. The amino acid segments can each independently comprise the amino acid sequence CREKA (SEQ ID NO: 1) or a conservative variant thereof.

The amino acid segments can also each independently comprise the amino acid sequence CREKA (SEQ ID NO:1). The amino acid segment can also consist of the amino acid sequence CREKA (SEQ ID NO:1). The amino acid segment can consist of the amino acid sequence REK.

b. Fibrin Binding Peptides

The clot-binding head group can also comprise a fibrin-binding peptide (FBP). Examples of fibrin-binding peptides are known in the art (Van Rooijen N, Sanders A (1994) J Immunol Methods 174: 83-93; Moghimi S M, Hunter A C, Murray J C (2001) Pharmacol Rev 53: 283-318; U.S. Pat. No. 5,792,742, all herein incorporated by reference in their entirety for their teaching concerning fibrin binding peptides).

c. Other Clot-Binding Peptides

Clot-binding peptides can also bind to proteins other than fibrin. Example include peptides that bind to fibronectin that has become incorporated into a clot (Pilch et al., (2006) PNAS, 103: 2800-2804, hereby incorporated in its entirety for its teaching concerning clot-binding peptides). An example of clot-binding peptides include, but is not limited to, CGLIIQKNEC (CLT1, SEQ ID NO: 2) and CNAGESSKNC (CLT2, SEQ ID NO: 3). The amino acid segments can also be independently selected from amino acid segments comprising the amino acid sequence CLT1 or CLT2 (SEQ ID NOs: 2 or 3) or a conservative variant thereof, amino acid segments comprising the amino acid sequence CLT1 or CLT2 (SEQ ID NOs: 2 or 3), or amino acid segments consisting of the amino acid sequence CLT1 or CLT2 (SEQ ID NOs: 2 or 3). The amino acid segments can each independently comprise the amino acid sequence CLT1 or CLT2 (SEQ ID NOs: 2 or 3) or a conservative variant thereof.

The amino acid segments can also each independently comprise the amino acid sequence CLT1 or CLT2 (SEQ ID NOS: 2 or 3). The amino acid segment can also consist of the amino acid sequence CLT1 or CLT2 (SEQ ID NOS: 2 or 3).

ii. Clot-Binding Antibodies

The clot-binding head group can comprise a clot-binding antibody. Examples of clot-binding antibodies are known in the art (Holvoet et al. Circulation, Vol 87, 1007-1016, 1993; Bode et al. J. Biol. Chem., Vol. 264, Issue 2, 944-948, January 1989; Huang et al. Science 1997: Vol. 275. no. 5299, pp. 547-550, all of which are herein incorporated by reference in their entirety for their teaching concerning clot-binding antibodies).

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to bind to, or otherwise interact with, clots. The antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the HIV Env-CD4-co-receptor complexes described herein.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

Human antibodies can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies (and fragments thereof) can also be produced using phage display libraries (Hoogenboom et al., J. Mol. Biol., 227:381, 1991; Marks et al., J. Mol. Biol., 222:581, 1991).

Human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5, 939,598 (Kucherlapati et al.), U.S. Pat. No. 6,130,364 (Jakobovits et al.), and U.S. Pat. No. 6,180,377 (Morgan et al.).

iii. Small Organic Molecules

The clot-binding head group can also be a small organic molecule. Small organic molecules that are capable of interacting with, or binding to, clots are known in the art. These molecules can also be identified by methods known in the art, such as combinatorial chemistry. Some forms of small organic molecules can be organic molecules having a molecular weight of less than 1000 Daltons.

Combinatorial chemistry includes but is not limited to all methods for isolating small molecules that are capable of interacting with a clot, molecules associated with a clot such as fibrin or fibronectin, or clotted plasma protein, for example. One synthesizes a large pool of molecules and subjects that complex mixture to some selection and enrichment process, such as the detection of an interaction with clots.

Using methodology well known to those of skill in the art, in combination with various combinatorial libraries, one can isolate and characterize those small molecules which bind to or interact with the desired target. The relative binding affinity of these compounds can be compared and optimum compounds identified using competitive binding studies, which are well known to those of skill in the art. For example, a competitive binding study using CREKA (SEQ ID NO: 1) can be used.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

As used herein combinatorial methods and libraries included traditional screening methods and libraries as well as methods and libraries used in iterative processes.

Libraries of small organic molecules generally comprise at least 2 organic compounds, often at least about 25, 100 500 different organic compounds, more usually at least about 1000 different organic compounds, preferably at least about 2500 different organic compounds, more preferably at least about 5000 different organic compounds and most preferably at least about 10,000 or more different organic compounds. Libraries may be selected or constructed such that each individual molecule of the library may be spatially separated from the other molecules of the library (e.g., each member of the library is present in a separate microtiter well) or two or more members of the library may be combined if methods for deconvolution are readily available. The methods by which the library of organic compounds are prepared are not critical.

2. Treatment Head Groups

The head group can be a treatment head group. As used herein, the term “treatment head group” means a molecule which has one or more biological activities in a normal or pathologic tissue. A variety of treatment head groups can be used as a head group.

In some embodiments, the treatment head group can be a cancer chemotherapeutic agent. As used herein, a “cancer chemotherapeutic agent” is a chemical agent that inhibits the proliferation, growth, life-span or metastatic activity of cancer cells. Such a cancer chemotherapeutic agent can be, without limitation, a taxane such as docetaxel; an anthracyclin such as doxorubicin; an alkylating agent; a vinca alkaloid; an anti-metabolite; a platinum agent such as cisplatin or carboplatin; a steroid such as methotrexate; an antibiotic such as adriamycin; a isofamide; or a selective estrogen receptor modulator; an antibody such as trastuzumab.

Taxanes are chemotherapeutic agents useful with the compositions disclosed herein. Useful taxanes include, without limitation, docetaxel (Taxotere; Aventis Pharmaceuticals, Inc.; Parsippany, N.J.) and paclitaxel (Taxol; Bristol-Myers Squibb; Princeton, N.J.). See, for example, Chan et al., J. Clin. Oncol. 17:2341-2354 (1999), and Paridaens et al., J. Clin. Oncol. 18:724 (2000).

A cancer chemotherapeutic agent useful with the compositions disclosed herein also can be an anthracyclin such as doxorubicin, idarubicin or daunorubicin. Doxorubicin is a commonly used cancer chemotherapeutic agent and can be useful, for example, for treating breast cancer (Stewart and Ratain, In: “Cancer: Principles and practice of oncology” 5th ed., chap. 19 (eds. DeVita, Jr., et al.; J. P. Lippincott 1997); Harris et al., In “Cancer: Principles and practice of oncology,” supra, 1997). In addition, doxorubicin has anti-angiogenic activity (Folkman, Nature Biotechnology 15:510 (1997); Steiner, In “Angiogenesis: Key principles-Science, technology and medicine,” pp. 449-454 (eds. Steiner et al.; Birkhauser Verlag, 1992)), which can contribute to its effectiveness in treating cancer.

An alkylating agent such as melphalan or chlorambucil also can be a useful cancer chemotherapeutic agent. Similarly, a vinca alkaloid such as vindesine, vinblastine or vinorelbine; or an antimetabolite such as 5-fluorouracil, 5-fluorouridine or a derivative thereof can be a useful cancer chemotherapeutic agent.

A platinum agent also can be a useful cancer chemotherapeutic agent. Such a platinum agent can be, for example, cisplatin or carboplatin as described, for example, in Crown, Seminars in Oncol. 28:28-37 (2001). Other useful cancer chemotherapeutic agents include, without limitation, methotrexate, mitomycin-C, adriamycin, ifosfamide and ansamycins.

A cancer chemotherapeutic agent useful for treatment of breast cancer and other hormonally-dependent cancers also can be an agent that antagonizes the effect of estrogen, such as a selective estrogen receptor modulator or an anti-estrogen. The selective estrogen receptor modulator, tamoxifen, is a cancer chemotherapeutic agent that can be used in a composition for treatment of breast cancer (Fisher et al., J. Natl. Cancer Instit. 90:1371-1388 (1998)).

The treatment head group can be an antibody such as a humanized monoclonal antibody. As an example, the anti-epidermal growth factor receptor 2 (HER2) antibody, trastuzumab (Herceptin; Genentech, South San Francisco, Calif.) can be a treatment head group useful for treating HER2/neu overexpressing breast cancers (White et al., Annu. Rev. Med. 52:125-141 (2001)).

Useful treatment head groups also can be a cytotoxic agent, which, as used herein, can be any molecule that directly or indirectly promotes cell death. Useful cytotoxic agents include, without limitation, small molecules, polypeptides, peptides, peptidomimetics, nucleic acid-molecules, cells and viruses. As non-limiting examples, useful cytotoxic agents include cytotoxic small molecules such as doxorubicin, docetaxel or trastuzumab; antimicrobial peptides such as those described further below; pro-apoptotic polypeptides such as caspases and toxins, for example, caspase-8; diphtheria toxin A chain, Pseudomonas exotoxin A, cholera toxin, ligand fusion toxins such as DAB389EGF, ricinus communis toxin (ricin); and cytotoxic cells such as cytotoxic T cells. See, for example, Martin et al., Cancer Res. 60:3218-3224 (2000); Kreitman and Pastan, Blood 90:252-259 (1997); Allam et al., Cancer Res. 57:2615-2618 (1997); and Osborne and Coronado-Heinsohn, Cancer J. Sci. Am. 2:175 (1996). One skilled in the art understands that these and additional cytotoxic agents described herein or known in the art can be useful in the disclosed compositions and methods.

In one embodiment, a treatment head group can be a therapeutic polypeptide. As used herein, a therapeutic polypeptide can be any polypeptide with a biologically useful function. Useful therapeutic polypeptides encompass, without limitation, cytokines, antibodies, cytotoxic polypeptides; pro-apoptotic polypeptides; and anti-angiogenic polypeptides. As non-limiting examples, useful therapeutic polypeptides can be a cytokine such as tumor necrosis factor-α (TNF-α), tumor necrosis factor-β (TNF-β), granulocyte macrophage colony stimulating factor (GM-CSF), granulocyte colony stimulating factor (G-CSF), interferon .alpha. (IFN-α); interferon .gamma. (IFN-γ), interleukin-1 (IL-1), interleukin-2 (IL-2), interleukin-3 (IL-3), interleukin-4 (IL-4), interleukin-6 (IL-6), interleukin-7 (IL-7), interleukin-10 (IL-10), interleukin-12 (IL-12), lymphotactin (LTN) or dendritic cell chemokine 1 (DC-CK1); an anti-HER2 antibody or fragment thereof; a cytotoxic polypeptide including a toxin or caspase, for example, diphtheria toxin A chain, Pseudomonas exotoxin A, cholera toxin, a ligand fusion toxin such as DAB389EGF or ricin; or an anti-angiogenic polypeptide such as angiostatin, endostatin, thrombospondin, platelet factor 4; anastellin; or one of those described further herein or known in the art (see below). It is understood that these and other polypeptides with biological activity can be a “therapeutic polypeptide.”

A treatment head group can also be an anti-angiogenic agent. As used herein, the term “anti-angiogenic agent” means a molecule that reduces or prevents angiogenesis, which is the growth and development of blood vessels. A variety of anti-angiogenic agents can be prepared by routine methods. Such anti-angiogenic agents include, without limitation, small molecules; proteins such as dominant negative forms of angiogenic factors, transcription factors and antibodies; peptides; and nucleic acid molecules including ribozymes, antisense oligonucleotides, and nucleic acid molecules encoding, for example, dominant negative forms of angiogenic factors and receptors, transcription factors, and antibodies and antigen-binding fragments thereof. See, for example, Hagedorn and Bikfalvi, Crit. Rev. Oncol. Hematol. 34:89-110 (2000), and Kirsch et al., J. Neurooncol. 50:149-163 (2000).

Vascular endothelial growth factor (VEGF) has been shown to be important for angiogenesis in many types of cancer, including breast cancer angiogenesis in vivo (Borgstrom et al., Anticancer Res. 19:4213-4214 (1999)). The biological effects of VEGF include stimulation of endothelial cell proliferation, survival, migration and tube formation, and regulation of vascular permeability. An anti-angiogenic agent can be, for example, an inhibitor or neutralizing antibody that reduces the expression or signaling of VEGF or another angiogenic factor, for example, an anti-VEGF neutralizing monoclonal antibody (Borgstrom et al., supra, 1999). An anti-angiogenic agent also can inhibit another angiogenic factor such as a member of the fibroblast growth factor family such as FGF-1 (acidic), FGF-2 (basic), FGF-4 or FGF-5 (Slavin et al., Cell Biol. Int. 19:431-444 (1995); Folkman and Shing, J. Biol. Chem. 267:10931-10934 (1992)) or an angiogenic factor such as angiopoietin-1, a factor that signals through the endothelial cell-specific Tie2 receptor tyrosine kinase (Davis et al., Cell 87:1161-1169 (1996); and Suri et al., Cell 87:1171-1180 (1996)), or the receptor of one of these angiogenic factors. It is understood that a variety of mechanisms can act to inhibit activity of an angiogenic factor including, without limitation, direct inhibition of receptor binding, indirect inhibition by reducing secretion of the angiogenic factor into the extracellular space, or inhibition of expression, function or signaling of the angiogenic factor.

A variety of other molecules also can function as anti-angiogenic agents including, without limitation, angiostatin; a kringle peptide of angiostatin; endostatin; anastellin, heparin-binding fragments of fibronectin; modified forms of antithrombin; collagenase inhibitors; basement membrane turnover inhibitors; angiostatic steroids; platelet factor 4 and fragments and peptides thereof; thrombospondin and fragments and peptides thereof; and doxorubicin (O'Reilly et al., Cell 79:315-328 (1994)); O'Reilly et al., Cell 88:277-285 (1997); Homandberg et al., Am. J. Path. 120:327-332 (1985); Homandberg et-al., Biochim. Biophys. Acta 874:61-71 (1986); and O'Reilly et al., Science 285:1926-1928 (1999)). Commercially available anti-angiogenic agents include, for example, angiostatin, endostatin, metastatin and 2ME2 (EntreMed; Rockville, Md.); anti-VEGF antibodies such as Avastin (Genentech; South San Francisco, Calif.); and VEGFR-2 inhibitors such as SU5416, a small molecule inhibitor of VEGFR-2 (SUGEN; South San Francisco, Calif.) and SU6668 (SUGEN), a small molecule inhibitor of VEGFR-2, platelet derived growth factor and fibroblast growth factor I receptor. It is understood that these and other anti-angiogenic agents can be prepared by routine methods and are encompassed by the term “anti-angiogenic agent” as used herein.

The compositions disclosed herein can also be used at a site of inflammation or injury. Head groups useful for this purpose can include treatment head groups belonging to several basic groups including anti-inflammatory agents which prevent inflammation, restenosis preventing drugs which prevent tissue growth, anti-thrombogenic drugs which inhibit or control formation of thrombus or thrombolytics, and bioactive agents which regulate tissue growth and enhance healing of the tissue. Examples of useful treatment head groups include but are not limited to steroids, fibronectin, anti-clotting drugs, anti- platelet function drugs, drugs which prevent smooth muscle cell growth on inner surface wall of vessel, heparin, heparin fragments, aspirin, coumadin, tissue plasminogen activator (TPA), urokinase, hirudin, streptokinase, antiproliferatives (methotrexate, cisplatin, fluorouracil, Adriamycin), antioxidants (ascorbic acid, beta carotene, vitamin E), antimetabolites, thromboxane inhibitors, non-steroidal and steroidal anti-inflammatory drugs, beta and calcium channel blockers, genetic materials including DNA and RNA fragments, complete expression genes, antibodies, lymphokines, growth factors, prostaglandins, leukotrienes, laminin, elastin, collagen, and integrins.

Useful treatment head groups also can be antimicrobial peptides. This can be particularly useful to target a wound or other infected sites. Thus, for example, also disclosed are head groups comprising an antimicrobial peptide, where the composition is selectively internalized and exhibits a high toxicity to the targeted area. Useful antimicrobial peptides can have low mammalian cell toxicity when not incorporated into the composition. As used herein, the term “antimicrobial peptide” means a naturally occurring or synthetic peptide having antimicrobial activity, which is the ability to kill or slow the growth of one or more microbes. An antimicrobial peptide can, for example, kill or slow the growth of one or more strains of bacteria including a Gram-positive or Gram-negative bacteria, or a fungi or protozoa. Thus, an antimicrobial peptide can have, for example, bacteriostatic or bacteriocidal activity against, for example, one or more strains of Escherichia coli, Pseudomonas aeruginosa or Staphylococcus aureus. While not wishing to be bound by the following, an antimicrobial peptide can have biological activity due to the ability to form ion channels through membrane bilayers as a consequence of self-aggregation.

An antimicrobial peptide is typically highly basic and can have a linear or cyclic structure. As discussed further below, an antimicrobial peptide can have an amphipathic α-helical structure (see U.S. Pat. No. 5,789,542; Javadpour et al., J. Med. Chem. 39:3107-3113 (1996); and Blondelle and Houghten, Biochem. 31: 12688-12694 (1992)). An antimicrobial peptide also can be, for example, a β-strand/sheet-forming peptide as described in Mancheno et al., J. Peptide Res. 51:142-148 (1998).

An antimicrobial peptide can be a naturally occurring or synthetic peptide. Naturally occurring antimicrobial peptides have been isolated from biological sources such as bacteria, insects, amphibians, and mammals and are thought to represent inducible defense proteins that can protect the host organism from bacterial infection. Naturally occurring antimicrobial peptides include the gramicidins, magainins, mellitins, defensins and cecropins (see, for example, Maloy and Kari, Biopolymers 37:105-122 (1995); Alvarez-Bravo et al., Biochem. J. 302:535-538 (1994); Bessalle et al., FEBS 274:-151-155 (1990.); and Blondelle and Houghten in Bristol (Ed.), Annual Reports in Medicinal Chemistry pages 159-168 Academic Press, San Diego). An antimicrobial peptide also can be an analog of a natural peptide, especially one that retains or enhances amphipathicity (see below).

An antimicrobial peptide incorporated into the composition disclosed herein can have low mammalian cell toxicity when linked to the composition. Mammalian cell toxicity readily can be assessed using routine assays. As an example, mammalian cell toxicity can be assayed by lysis of human erythrocytes in vitro as described in Javadpour et al., supra, 1996. An antimicrobial peptide having low mammalian cell toxicity is not lytic to human erythrocytes or requires concentrations of greater than 100 μM for lytic activity, preferably concentrations greater than 200, 300, 500 or 1000 μM.

In one embodiment, disclosed are compositions in which the antimicrobial peptide portion promotes disruption of mitochondrial membranes when internalized by eukaryotic cells. In particular, such an antimicrobial peptide preferentially disrupts mitochondrial membranes as compared to eukaryotic membranes. Mitochondrial membranes, like bacterial membranes but in contrast to eukaryotic plasma membranes, have a high content of negatively charged phospholipids. An antimicrobial peptide can be assayed for activity in disrupting mitochondrial membranes using, for example, an assay for mitochondrial swelling or another assay well known in the art.

An antimicrobial peptide that induces significant mitochondrial swelling at, for example, 50 μM, 40 μM, 30 μM, 20 μM, 10 μM, or less, is considered a peptide that promotes disruption of mitochondrial membranes.

Antimicrobial peptides generally have random coil conformations in dilute aqueous solutions, yet high levels of helicity can be induced by helix-promoting solvents and amphipathic media such as micelles, synthetic bilayers or cell membranes. α-Helical structures are well known in the art, with an ideal α-helix characterized by having 3.6 residues per turn and a translation of 1.5 Å per residue (5.4 Å per turn; see Creighton, Proteins: Structures and Molecular Properties W. H Freeman, New York (1984)). In an amphipathic α-helical structure, polar and non-polar amino acid residues are aligned into an amphipathic helix, which is an α-helix in which the hydrophobic amino acid residues are predominantly on one face, with hydrophilic residues predominantly on the opposite face when the peptide is viewed along the helical axis.

Antimicrobial peptides of widely varying sequence have been isolated, sharing an amphipathic α-helical structure as a common feature (Saberwal et al., Biochim. Biophys. Acta 1197:109-131 (1994)). Analogs of native peptides with amino acid substitutions predicted to enhance amphipathicity and helicity typically have increased antimicrobial activity. In general, analogs with increased antimicrobial activity also have increased cytotoxicity against mammalian cells (Maloy et al., Biopolymers 37:105-122 (1995)).

As used herein in reference to an antimicrobial peptide, the term “amphipathic α-helical structure” means an α-helix with a hydrophilic face containing several polar residues at physiological pH and a hydrophobic face containing nonpolar residues. A polar residue can be, for example, a lysine or arginine residue, while a nonpolar residue can be, for example, a leucine or alanine residue. An antimicrobial peptide having an amphipathic .alpha.-helical structure generally has an equivalent number of polar and nonpolar residues within the amphipathic domain and a sufficient number of basic residues to give the peptide an overall positive charge at neutral pH (Saberwal et al., Biochim. Biophys. Acta 1197:109-131 (1994)). One skilled in the art understands that helix-promoting amino acids such as leucine and alanine can be advantageously included in an antimicrobial peptide (see, for example, Creighton, supra, 1984). Synthetic, antimicrobial peptides having an amphipathic α-helical structure are known in the art, for example, as described in U.S. Pat. No. 5,789,542 to McLaughlin and Becker.

It is understood by one skilled in the art of medicinal oncology that these and other agents are useful treatment head groups, which can be used separately or together in the disclosed compositions and methods. Thus, it is understood that the compositions disclosed herein can contain one or more of such treatment head groups and that additional components can be included as part of the composition, if desired. As a non-limiting example, it can be desirable in some cases to utilize an oligopeptide spacer between the clot-binding head group and the treatment head group (Fitzpatrick and Garnett, Anticancer Drug Des. 10:1-9 (1995)).

Other useful agents include thrombolytics, aspirin, anticoagulants, painkillers and tranquilizers, beta-blockers, ace-inhibitors, nitrates, rhythm-stabilizing drugs, and diuretics. Agents that limit damage to the heart work best if given within a few hours of the heart attack. Thrombolytic agents that break up blood clots and enable oxygen-rich blood to flow through the blocked artery increase the patient's chance of survival if given as soon as possible after the heart attack. Thrombolytics given within a few hours after a heart attack are the most effective. Injected intravenously, these include anisoylated plasminogen streptokinase activator complex (APSAC) or anistreplase, recombinant tissue-type plasminogen activator (r-tPA), and streptokinase. The disclosed compounds can use any of these or similar agents.

3. Detection Head Groups

The head group in the disclosed compositions can also be a detection head group. A variety of detection head groups are useful in the disclosed methods. As used herein, the term “detection head group” refers to any molecule which can be detected. Useful detection head groups include compounds and molecules that can be administered in vivo and subsequently detected. Detection head groups useful in the disclosed compositions and methods include yet are not limited to radiolabels and fluorescent molecules. The detection head group can be, for example, any molecule that facilitates detection, either directly or indirectly, preferably by a non-invasive and/or in vivo visualization technique. For example, a detection head group can be detectable by any known imaging techniques, including, for example, a radiological technique, a magnetic resonance technique, or an ultrasound technique. Detection head groups can include, for example, a contrasting agent, e.g., where the contrasting agent is ionic or non-ionic. In some embodiments, for instance, the detection head group comprises a tantalum compound and/or a barium compound, e.g., barium sulfate. In some embodiments, the detection head group comprises iodine, such as radioactive iodine. In some embodiments, for instance, the detection head group comprises an organic iodo acid, such as iodo carboxylic acid, triiodophenol, iodoform, and/or tetraiodoethylene. In some embodiments, the detection head group comprises a non-radioactive detection head group, e.g., a non-radioactive isotope. For example, Gd can be used as a non-radioactive detection head group in certain embodiments.

Other examples of detection head groups include molecules which emit or can be caused to emit detectable radiation (e.g., fluorescence excitation, radioactive decay, spin resonance excitation, etc.), molecules which affect local electromagnetic fields (e.g., magnetic, ferromagnetic, ferromagnetic, paramagnetic, and/or superparamagnetic species), molecules which absorb or scatter radiation energy (e.g., chromophores and/or fluorophores), quantum dots, heavy elements and/or compounds thereof. See, e.g., detectable agents described in U.S. Publication No. 2004/0009122. Other examples of detection head groups include a proton-emitting molecules, a radiopaque molecules, and/or a radioactive molecules, such as a radionuclide like Tc-99m and/or Xe-13. Such molecules can be used as a radiopharmaceutical. In still other embodiments, the disclosed compositions can comprise one or more different types of detection head groups, including any combination of the detection head groups disclosed herein.

Useful fluorescent head groups include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions such as quantum dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin EBG, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Particularly useful fluorescent labels include fluorescein (5-carboxyfluorescein-N-hydroxysuccinimide ester), rhodamine (5,6-tetramethyl rhodamine), and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio. Fluorescent probes and there use are also described in Handbook of Fluorescent Probes and Research Products by Richard P. Haugland.

Further examples of radioactive detection head groups include gamma emitters, e.g., the gamma emitters In-111, I-125 and I-131, Rhenium-186 and 188, and Br-77 (see. e.g., Thakur, M. L. et al., Throm Res. Vol. 9 pg. 345 (1976); Powers et al., Neurology Vol. 32 pg. 938 (1982); and U.S. Pat. No. 5,011,686); positron emitters, such as Cu-64, C-11, and O-15, as well as Co-57, Cu-67, Ga-67, Ga-68, Ru-97, Tc-99m, In-113m, Hg-197, Au-198, and Pb-203. Other radioactive detection head groups can include, for example tritium, C-14 and/or thallium, as well as Rh-105, I-123, Nd-147, Pm-151, Sm-153, Gd-159, Tb-161, Er-171 and/or Tl-201.

The use of Technitium-99m (Tc-99m) is preferable and has been described in other applications, for example, see U.S. Pat. No. 4,418,052 and U.S. Pat. No. 5,024,829. Tc-99m is a gamma emitter with single photon energy of 140 keV and a half-life of about 6 hours, and can readily be obtained from a Mo-99/Tc-99 generator.

In some embodiments, compositions comprising a radioactive detection head group can be prepared by coupling a targeting head group with radioisotopes suitable for detection. Coupling can occur via a chelating agent such as diethylenetriaminepentaacetic acid (DTPA), 4,7,10-tetraazacyclododecane-N-,N′,N″,N′″-tetraacetic acid (DOTA) and/or metallothionein, any of which can be covalently attached to the targeting head group. In some embodiments, an aqueous mixture of technetium-99m, a reducing agent, and a water-soluble ligand can be prepared and then allowed to react with a disclosed targeting head group. Such methods are known in the art, see e.g., International Publication No. WO 99/64446. In some embodiments, compositions comprising radioactive iodine, can be prepared using an exchange reaction. For example, exchange of hot iodine for cold iodine is well known in the art. Alternatively, a radio-iodine labeled compound can be prepared from the corresponding bromo compound via a tributylstannyl intermediate.

Magnetic detection head groups include paramagnetic contrasting agents, e.g., gadolinium diethylenetriaminepentaacetic acid, e.g., used with magnetic resonance imaging (MRI) (see, e.g., De Roos, A. et al., Int. J. Card. Imaging Vol. 7 pg. 133 (1991)). Some preferred embodiments use as the detection head group paramagnetic atoms that are divalent or trivalent ions of elements with an atomic number 21, 22, 23, 24, 25, 26, 27, 28, 29, 42, 44, 58, 59, 60, 61, 62, 63, 64, 65, 66, 67, 68, 69, or 70. Suitable ions include, but are not limited to, chromium(III), manganese(II), iron(II), iron(III), cobalt(II), nickel(II), copper(II), praseodymium(III), neodymium(III), samarium(III) and ytterbium(III), as well as gadolinium(III), terbiurn(III), dysoprosium(III), holmium(III), and erbium(III). Some preferred embodiments use atoms with strong magnetic moments, e.g., gadolinium(III).

In some embodiments, compositions comprising magnetic detection head groups can be prepared by coupling a targeting head group with a paramagnetic atom. For example, the metal oxide or a metal salt, such as a nitrate, chloride or sulfate salt, of a suitable paramagnetic atom can be dissolved or suspended in a water/alcohol medium, such as methyl, ethyl, and/or isopropyl alcohol. The mixture can be added to a solution of an equimolar amount of the targeting head group in a similar water/alcohol medium and stirred. The mixture can be heated moderately until the reaction is complete or nearly complete. Insoluble compositions formed can be obtained by filtering, while soluble compositions can be obtained by evaporating the solvent. If acid groups on the chelating head groups remain in the disclosed compositions, inorganic bases (e.g., hydroxides, carbonates and/or bicarbonates of sodium, potassium and/or lithium), organic bases, and/or basic amino acids can be used to neutralize acidic groups, e.g., to facilitate isolation or purification of the composition.

In preferred embodiments, the detection head group can be coupled to the composition in such a way so as not to interfere with the ability of the clot-binding head group to interact with the clotting site. In some embodiments, the detection head group can be chemically bound to the clot-binding head group. In some embodiments, the detection head group can be chemically bound to a head group that is itself chemically bound to the clot-binding head group, indirectly linking the imaging and targeting head groups.

### C. Pharmaceutical Compositions and Carriers

The disclosed compositions can be administered in vivo either alone or in a pharmaceutically acceptable carrier. By “pharmaceutically acceptable” is meant a material that is not biologically or otherwise undesirable, i.e., the material can be administered to a subject, along with the composition disclosed herein, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. The materials can be in solution, suspension (for example, incorporated into microparticles, liposomes, or cells).

1. Pharmaceutically Acceptable Carriers

The compositions disclosed herein can be used therapeutically in combination with a pharmaceutically acceptable carrier.

Suitable carriers and their formulations are described in Remington: The Science and Practice of Pharmacy (19th ed.) ed. A.R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers can be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### D. Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, peptides, etc., are also disclosed. Thus, the products produced using the molecular modeling approaches that involve the disclosed compositions are also considered herein disclosed.

Thus, one way to isolate molecules that bind a molecule of choice is through rational design. This can be achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol._{—}Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

E. Compositions with Similar Functions

It is understood that the compositions disclosed herein have certain functions, such as binding to clots or enhancing clot formation. Disclosed herein are certain structural requirements for performing the disclosed functions, and it is understood that there are a variety of structures which can perform the same function which are related to the disclosed structures, and that these structures will ultimately achieve the same result, for example stimulation or inhibition.

### F. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits can include the compositions disclosed herein.

### G. Mixtures

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### H. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated.

### I. Computer Readable Media

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

### J. Peptide Synthesis

The compositions disclosed herein and the compositions necessary to perform the disclosed methods can be made using any method known to those of skill in the art for that particular reagent or compound unless otherwise specifically noted.

One method of producing the disclosed proteins, such as SEQ ID NO:1, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides can be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

### Methods

Disclosed herein is a method comprising administering to a subject the composition disclosed herein. The composition can selectively home to clotted plasma protein. Also disclosed are methods comprising administering a composition to a subject, wherein the composition comprises amphiphile molecules, wherein at least one of the amphiphile molecules comprises a clot-binding head group, wherein the clot-binding head group selectively binds to clotted plasma protein, wherein the composition does not cause clotting, wherein the composition binds to clotted plasma protein in the subject. Also disclosed are methods comprising administering one or more of the disclosed compositions to a subject, wherein the composition binds to clotted plasma protein in the subject.

Also disclosed are methods of making a composition, the method comprising mixing amphiphile molecules, wherein at least one of the amphiphile molecules comprises a clot-binding head group, wherein the clot-binding head group selectively binds to clotted plasma protein, and wherein the composition does not cause clotting. Also disclosed are methods of making a composition, the method comprising mixing amphiphile molecules, wherein at least one of the amphiphile molecules comprises one or more of the disclosed clot-binding head group.

The amphiphile molecules can comprise a functional head group. At least one of the amphiphile molecules can comprise a functional head group. The functional head group can be a detection head group. The functional head group can be a treatment head group. At least one of the amphiphile molecules can comprise a detection head group and at least one of the amphiphile molecules can comprise a treatment head group.

The subject can be in need of treatment of a disease or condition associated with and/or that produces clotted plasma protein. The subject can be in need of treatment of cardiovascular disease. The subject can be in need of detection, visualization, or both of a disease or condition associated with and/or that produces clotted plasma protein. The subject can be in need of detection, visualization, or both of cardiovascular disease. The subject can be in need of detection, visualization, or both of cancer, a tumor, or both. The subject can be in need of treatment of cancer.

Administering the composition can treat a disease or condition associated with and/or that produces clotted plasma protein. Administering the composition can treat a cardiovascular disease. The cardiovascular disease can be atherosclerosis. Administering the composition can treat cancer. The method can further comprise detecting, visualizing, or both the disease or condition associated with and/or that produces clotted plasma protein. The method can further comprise detecting, visualizing, or both the cardiovascular disease. The method can further comprise detecting, visualizing, or both the cancer, tumor, or both.

The method can further comprise, prior to administering, subjecting the amphiphile molecules to a hydrophilic medium. The amphiphile molecules can form an aggregate in the hydrophilic medium. The aggregate can comprise a micelle. The method can further comprise, following administering, detecting the amphiphile molecules. The amphiphile molecules can be detected by fluorescence, PET or MRI. The amphiphile molecules can be detected by fluorescence. The composition can conjugate with a plaque in a subject. The composition can conjugate with a tumor in a subject.

The clot-binding head groups can each be independently selected from an amino acid segment comprising the amino acid sequence REK, a fibrin-binding peptide, a clot-binding antibody, and a clot-binding small organic molecule. The clot-binding head groups can each independently comprise an amino acid segment comprising the amino acid sequence REK.

The clot-binding head groups can each comprise a fibrin-binding peptide. The fibrin-binding peptides can independently be selected from the group consisting of fibrin binding proteins and fibrin-binding derivatives thereof. In another example, the clot-binding head groups can each comprise a clot-binding antibody. Furthermore, the clot-binding head groups can each comprise a clot-binding small organic molecule.

The composition can further comprise a lipid, micelle, liposome, nanoparticle, microparticle, or fluorocarbon microbubble. In one example, the composition can be detectable. In another example, the composition can comprise a treatment head group. An example of a treatment head group is hirulog.

The composition can further comprise one or more head groups. For example, the head groups can be independently selected from the group consisting of an anti-angiogenic agent, a pro-angiogenic agent, a cancer chemotherapeutic agent, a cytotoxic agent, an anti-inflammatory agent, an anti-arthritic agent, a polypeptide, a nucleic acid molecule, a small molecule, a fluorophore, fluorescein, rhodamine, a radionuclide, indium-111, technetium-99, carbon-11, and carbon-13. At least one of the head groups can be a treatment head group. Examples of treatment head groups are paclitaxel and taxol. At least one of the head groups can be a detection head group.

The composition can selectively home to clotted plasma protein. The composition can selectively home to tumor vasculature, wound sites, or both. In one example, the composition can have a therapeutic effect. This effect can be enhanced by the delivery of a treatment head group to the site of the tumor or wound site.

The therapeutic effect can be a slowing in the increase of or a reduction of cardiovascular disease. The therapeutic effect can be a slowing in the increase of or a reduction of atherosclerosis. The therapeutic effect can be a slowing in the increase of or a reduction of the number and/or size of plaques. The therapeutic effect can be a reduction in the level or amount of the causes or symptoms of the disease being treated. The therapeutic effect can be a slowing in the increase of or a reduction of tumor burden.

The subject can have one or more sites to be targeted, wherein the composition homes to one or more of the sites to be targeted. For example, the subject can have multiple tumors or sites of injury.

In some forms, the composition can have a therapeutic effect. In some forms, this can be achieved by delivering a therapeutic compound or composition to the site of clotted plasma protein. This effect can be enhanced by the delivery of a treatment head group to the site of a tumor or wound site.

The therapeutic effect can be a slowing in the increase of or a reduction of cardiovascular disease. This slowing and/or reduction of the number and/or size of plaques can be 1%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, 600%, 700%, 800%, 900%, or 1000% or more improvement in the slowing and/or reduction of cardiovascular disease, compared with a non-treated subject, non-treated cardiovascular disease, a subject treated by a different method, or cardiovascular disease treated by a different method.

The therapeutic effect can be a slowing in the increase of or a reduction of atherosclerosis. This slowing and/or reduction of the number and/or size of plaques can be 1%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, 600%, 700%, 800%, 900%, or 1000% or more improvement in the slowing and/or reduction of atherosclerosis, compared with a non-treated subject, non-treated atherosclerosis, a subject treated by a different method, or atherosclerosis treated by a different method.

The therapeutic effect can be a slowing in the increase of or a reduction of the number and/or size of plaques. This slowing and/or reduction of the number and/or size of plaques can be 1%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, 600%, 700%, 800%, 900%, or 1000% or more improvement in the slowing and/or reduction of the number and/or size of plaques, compared with a non-treated subject, non-treated plaques, a subject treated by a different method, or plaques treated by a different method.

The therapeutic effect can be a reduction in the level or amount of the causes or symptoms of the disease being treated. This reduction in the level or amount of the causes or symptoms of the disease being treated can be 1%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, 600%, 700%, 800%, 900%, or 1000% or more improvement in the reduction in the level or amount of the causes or symptoms of the disease being treated, compared with a non-treated subject, non-treated disease, a subject treated by a different method, or the disease treated by a different method.

The therapeutic effect can be a slowing in the increase of or a reduction of tumor burden. This slowing in the increase of, or reduction in the tumor burden, can be 1%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, 600%, 700%, 800%, 900%, or 1000% or more improvement in the increase of, or reduction in the tumor burden of, compared with a non-treated tumor, or a tumor treated by a different method.

The subject can have one or more sites to be targeted, wherein the composition homes to one or more of the sites to be targeted. For example, the subject can have multiple tumors or sites of injury.

The disclosed compositions can be used to treat any disease where uncontrolled cellular proliferation occurs such as cancers. A non-limiting list of different types of cancers can be as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumors, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

The disclosed compositions can also be administered following decoy particle pretreatment to reduce uptake of the compositions by reticuloendothelial system (RES) tissues. Such decoy particle pretreatment can prolong the blood half-life of the particles and increases tumor targeting.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### A. Example 1

### Biomimetic Amplification of Nanoparticle Homing to Tumors

Targeted diagnostics and therapeutics are useful. Described herein are peptides that recognize clotted plasma proteins and selectively homes to sites of such clotted plasma proteins. Although this example describes homing to tumors and amplification of clotting, the disclosed peptides can be used to target diagnostics to other locations of clotted plasma proteins, such as sites of cardiovascular disease. Example 2 describes and example of such a use. The present example illustrates the targeting ability of a certain peptide. In this example, iron oxide nanoparticles and liposomes coated with this clotted plasma protein-homing peptide accumulate in tumor vessels, where they induce additional local clotting, thereby producing new binding sites for more particles. The system mimics platelets, which also circulate freely but accumulate at a diseased site and amplify their own accumulation at that site. The clotting-based amplification greatly enhances tumor imaging, and the addition of a drug carrier function to the particles can also be used.

1. Results

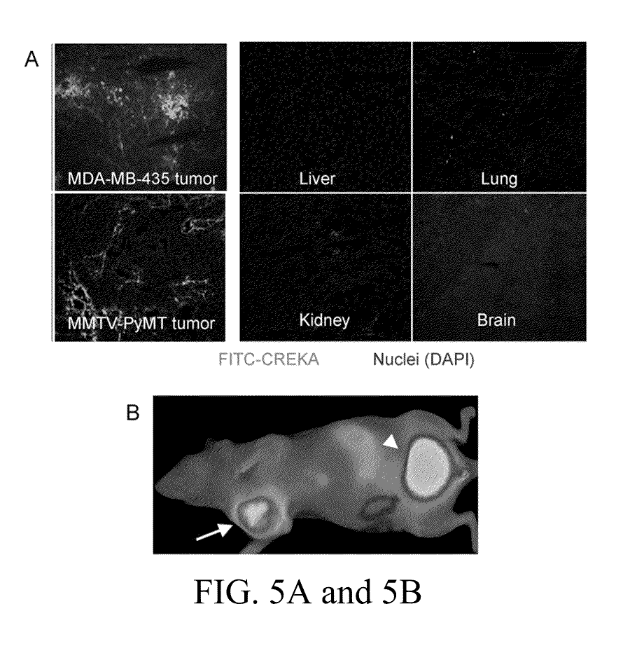

CREKA peptide. A tumor-homing peptide was used to construct targeted nanoparticles. This peptide was identified by in vivo screening of phage-displayed peptide libraries (Hoffman 2003; Pasqualini 1996) for tumor homing in tumor-bearing MMTV-PyMT transgenic breast cancer mice (Hutchinson 2000). The most frequently represented peptide sequence in the selected phage preparation was CREKA (cys-arg-glu-lys-ala; SEQ ID NO:1). The CREKA peptide was synthesized with a fluorescent dye attached to the N- terminus and the in vivo distribution of the peptide was studied in tumor-bearing mice. Intravenously injected CREKA peptide was readily detectable in the PyMT tumors, and in MDA-MB-435 human breast cancer xenografts, minutes to hours after the injection. The peptide formed a distinct meshwork in the tumor stroma (FIG. 5), and it also highlighted the blood vessels in the tumors. The CREKA peptide was essentially undetectable in normal tissues. In agreement with the microscopy results, whole body imaging using CREKA peptide labeled with the fluorescent dye Alexa 647 revealed peptide accumulation in the breast cancer xenografts, and in the bladder, reflecting elimination of excess peptide into the urine (FIG. 5B).

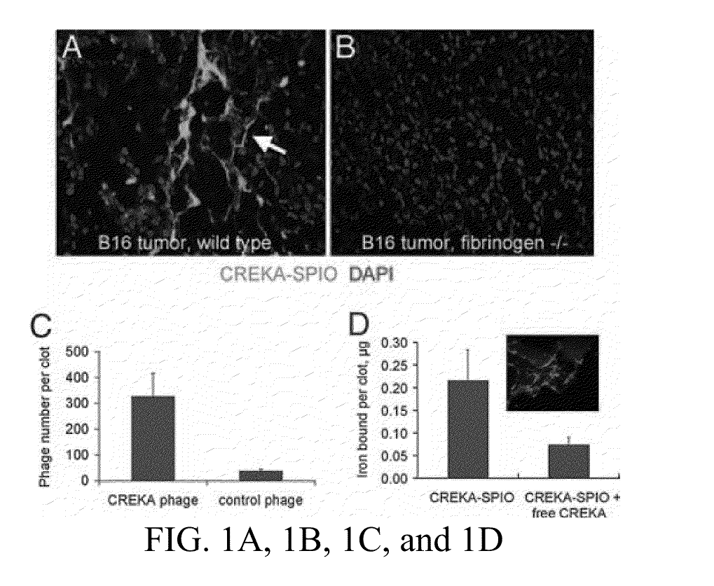

Tumors contain a meshwork of clotted plasma proteins in the tumor stroma and the walls of vessels, but no such meshwork is detectable in normal tissues (Dvorak 1985; Abe 1999; Pilch 2006). The mesh-like pattern produced by the CREKA peptide in tumors prompted the study of whether clotted plasma proteins can be the target of this peptide. The peptide was tested in fibrinogen knockout mice, which lack the fibrin meshwork in their tumors. Like previously identified clot-binding peptides (Pilch 2006), intravenously injected CREKA peptide failed to accumulate in B16F1 melanomas grown in the fibrinogen null mice, but formed a brightly fluorescent meshwork in B16F1 tumors grown in normal littermates of the null mice (FIGS. 1A and B). In agreement with this result, the CREKA phage, but not the control insertless phage, bound to clotted plasma proteins in vitro (FIG. 1C). These results establish CREKA as a clot-binding peptide. Its structure makes it an attractive peptide to use in nanoparticle targeting because, unlike other clot-binding peptides, which are cyclic 10 amino-acid peptides (Pilch 2006), CREKA is linear and contains only 5 amino acids. Moreover, the sulfhydryl group of the single cysteine residue is not required to provide binding activity and can be used to couple the peptide to other moieties.

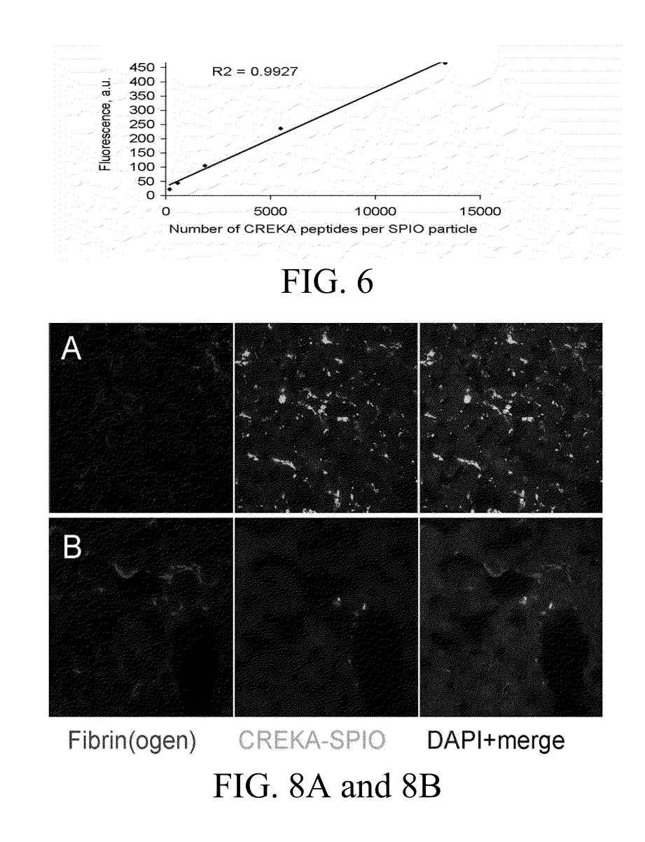

Peptide-coated nanoparticles. Fluorescein-labeled CREKA or fluorescein was coupled onto the surface of 50 nm superparamagnetic, amino dextran-coated iron oxide (SPIO) nanoparticles. Such particles have been extensively characterized with regard to their chemistry, pharmacokinetics, and toxicology, and are used as MRI contrast agents (Jung 1995; Jung 1995; Weissleder 1989). Coupling of the fluorescein-labeled peptides to SPIO produced strongly fluorescent particles. Releasing the peptide from the particles by hydrolysis increased the fluorescence further by a factor of about 3. These results indicate that the proximity of the fluorescein molecules at the particle surface causes some quenching of the fluorescence. Despite this, fluorescence from the coupled fluorescein peptide was almost linearly related to the number of peptide molecules on the particle (FIG. 6), allowing for the tracking of the number of peptide moieties on the particle by measuring particle fluorescence, and the use of fluorescence intensity as a measure of the concentration of particles in samples. CREKA-SPIO was used with at least 8,000 peptide molecules per particle in the in vivo experiments. The CREKA-SPIO nanoparticles bound to mouse and human plasma clots in vitro, and the binding was inhibited by the free peptide (FIG. 1D), The nanoparticles distributed along a fibrillar meshwork in the clots (inset in FIG. 1D). These results show that the particle-bound peptide retains its binding activity toward clotted plasma proteins.

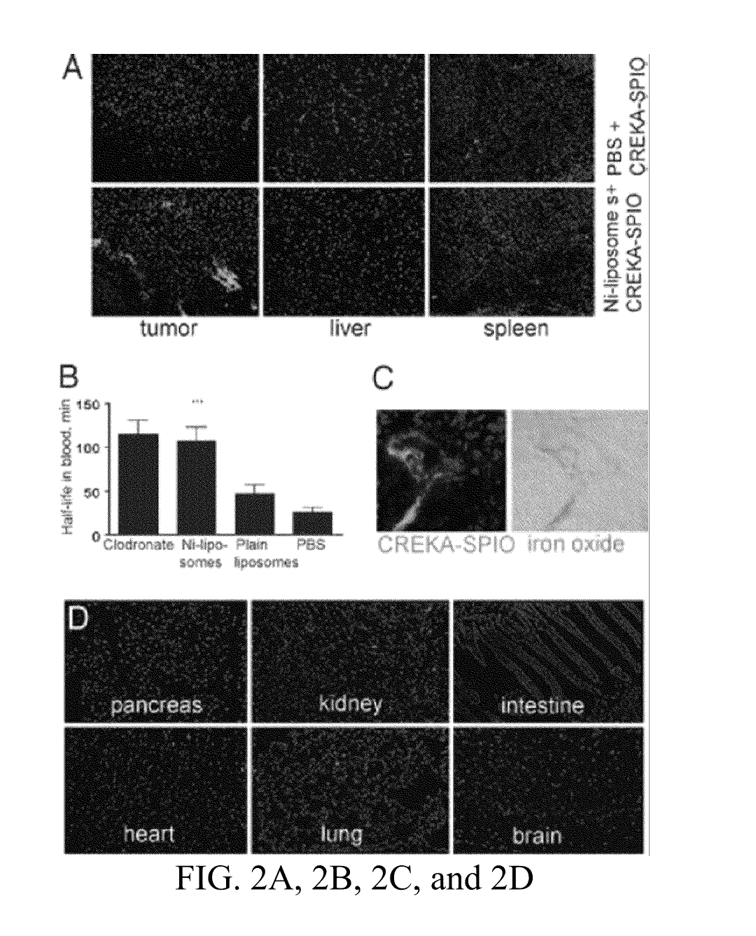

Tumor homing versus liver clearance of CREKA-SPIO. Initial experiments showed that intravenously injected CREKA-SPIO nanoparticles did not accumulate effectively in MDA-MB-435 breast cancer xenografts. In contrast, a high concentration of particles was seen in reticuloendothelial system (RES) tissues (FIG. 2A, upper panels). As the free CREKA peptide effectively homes to these tumors (FIG. 5), it was hypothesized that the RES uptake was a major obstacle to the homing of the nanoparticles. The role of the RES in the clearance of CREKA-SPIO was confirmed by depleting RES macrophages in the liver with liposomal clodronate (Van Rooijen 1994). This treatment caused about a 5-fold prolongation in particle half-life (FIG. 2B). Particulate material was eliminated from the circulation because certain plasma proteins bind to the particles and mediate their uptake by the RES (opsonization; Moghimi 2001; Moore 1997). Injecting decoy particles that eliminate plasma opsonins is another commonly used way of blocking RES uptake (Souhami 1981; Fernandez-Urrusuno 1996). Liposomes coated with chelated Ni²⁺ were tested as a potential decoy particle because it was surmised that iron oxide and Ni²⁺ would attract similar plasma opsonins, and Ni-liposomes could therefore deplete them from the systemic circulation. Indeed, SDS-PAGE analysis showed that significantly less plasma protein bound to SPIO in the blood of mice that had been pre-treated with Ni-liposomes.

Intravenously injected Ni-liposomes prolonged the half-life of the SPIO and CREKA-SPIO in the blood by a factor of about 5 (FIG. 2B). The Ni-liposome pretreatment whether done 5 min or 48 h prior to the injection of CREKA-SPIO, greatly increased the tumor homing of the nanoparticles, which primarily localized in tumor blood vessels (FIG. 2A lower tumor panel and FIG. 2D). The local concentration of particles was so high that the brownish color of iron oxide was visible in the optical microscope (FIG. 2C, right panel), indicating that the fluorescent signal observed in tumor vessels was from intact CREKA-SPIO. Fewer particles were seen in the liver after the Ni-liposome pre-treatment, but accumulation in the spleen was unchanged or even enhanced (FIG. 2A). Other organs contained minor amounts of CREKA-SPIO particles or no particles at all, whether Ni-liposomes were used or not (FIG. 1D). Plain liposomes were tested as decoy particles. These liposomes prolonged the blood half-life and tumor homing of subsequently injected CKEKA-SPIO (FIG. 2B), showing the existence of a common clearance mechanism for liposomes and SPIO.

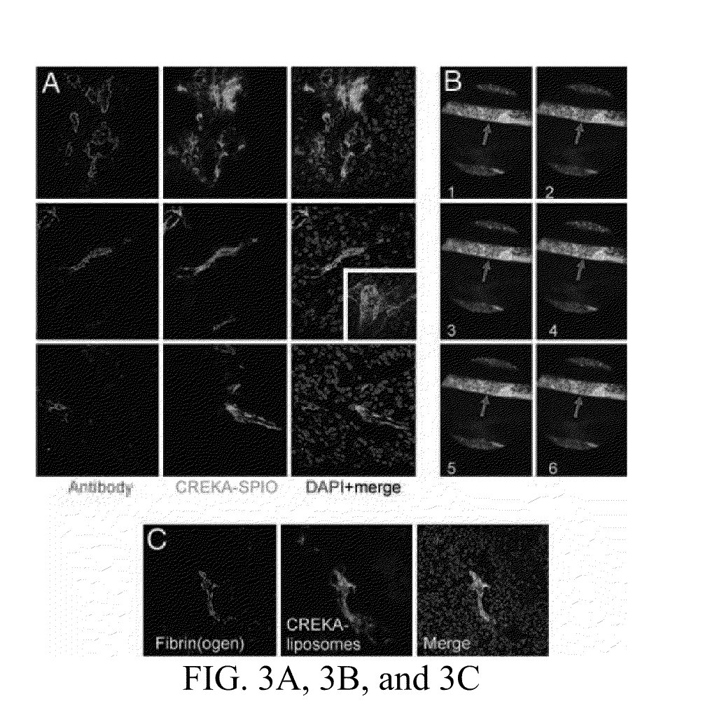

Nanoparticle-induced blood clotting in tumor vessels. CREKA-SPIO particles administered after liposome pretreatment primarily colocalized with tumor blood vessels, with some particles appearing to have extravasated into the surrounding tissue (FIG. 3A, top panels). Significantly, up to 20% of tumor vessel lumens were filled with fluorescent masses. These structures stained for fibrin (FIG. 3A, middle panels), showing that they are blood clots impregnated with nanoparticles. In some of the blood vessels the CREKA-SPIO nanoparticles were distributed along a meshwork (inset), possibly formed of fibrin and associated proteins, and similar to the pattern shown in the inset of FIG. 1D.

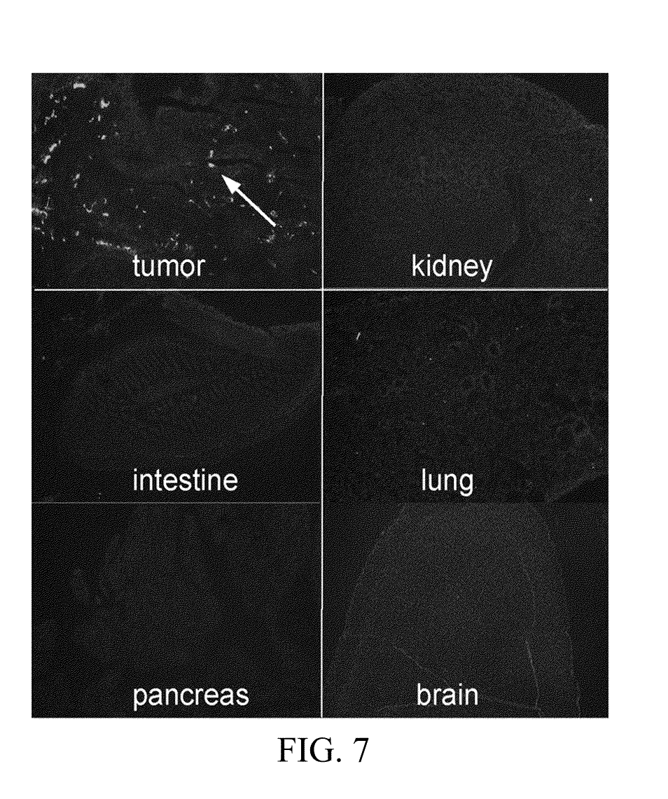

Among the non-RES tissues, the kidneys and lungs contained minor amounts of specific CREKA-SPIO fluorescence (FIG. 2D). However, low magnification images, which reveal only blood vessels with clots in them, showed no clotting in these tissues, with the exception of very rare clots in the kidneys (FIG. 7). Despite massive accumulation of nanoparticles in the liver no colocalization between fibrin(ogen) staining and CREKA-SPIO fluorescence in liver vessels (FIG. 8) was seen. Moreover, liver tissue from a non-injected mouse also stained for fibrin(ogen) (FIG. 8B), presumably reflecting fibrinogen production by hepatocytes. Thus, the clotting induced by CREKA-SPIO nanoparticles is essentially confined to tumor vessels.

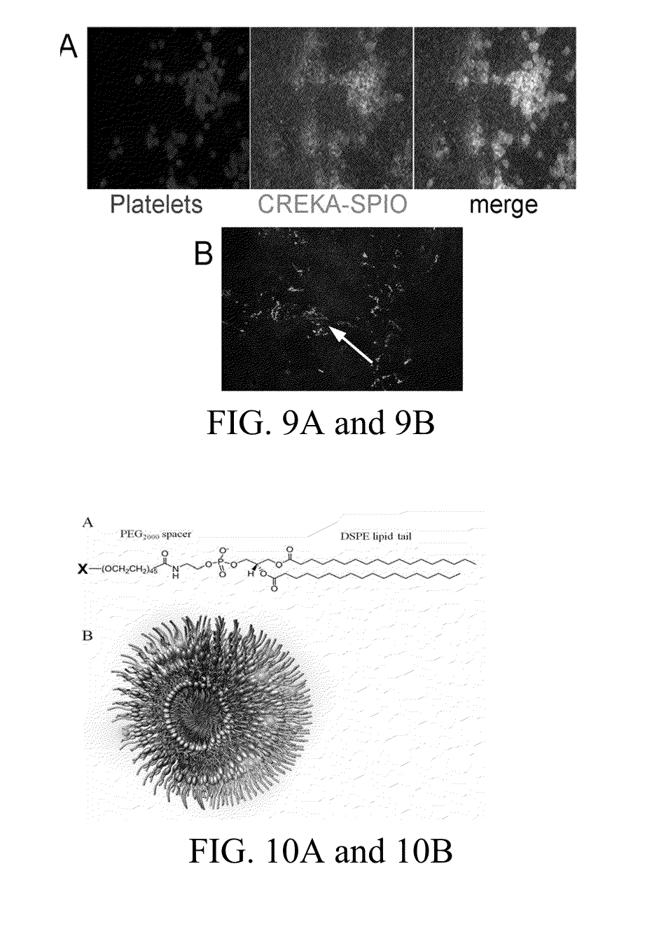

Nanoparticles can cause platelet activation and enhance thrombogenesis (Radomski 2005; Khandoga 2004). Some CREKA-SPIO nanoparticles (<1%) recovered from blood were associated with platelets (FIG. 9A), but staining for a platelet marker showed no colocalization between the platelets and CREKA-SPIO nanoparticles in tumor vessels (FIG. 3A, lower panels). Thrombocytopenia was also induced by injecting mice with an anti-CD41 monoclonal antibody (Van der Heyde 2005) and no noticeable effect on CREKA-SPIO homing to the MDA-MB-435 tumors was found (FIG. 9B). These results indicate that platelets are not involved in the homing pattern of CREKA-SPIO.

The deep infiltration of clots by nanoparticles showed that these clots must have formed at the time particles were circulating in blood, rather than before the injection. This was tested with intravital confocal microscopy, using DiI-labeled erythrocytes as a flow marker. There was time-dependent clot formation and obstruction of blood flow in tumor blood vessels with parallel entrapment of CREKA-SPIO in the forming clots (FIG. 3B).

It was next tested whether the clotting-inducing effect was specific for SPIO particles, or could be induced with a different CREKA-coated particle. Liposomes into which fluorescein-CREKA peptide was incorporated that was coupled to lipid-tailed polyethylene glycol (PEG) was used. Like CREKA-SPIO, the CREKA-liposomes selectively homed to tumors and co-localized with fibrin within tumor vessels (FIG. 3C), showing that CREKA liposomes are also capable of causing clotting in tumor vessels. No clotting was seen when control SPIO or control liposomes were injected in the tumor mice.

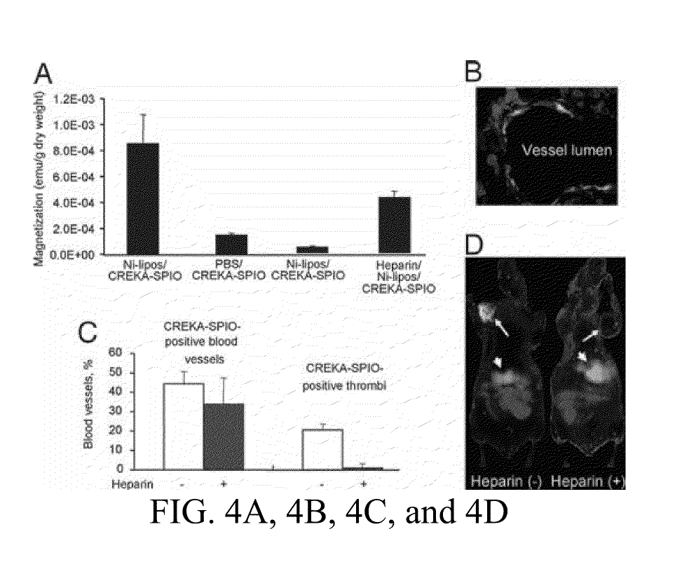

Clotting-amplified tumor targeting. The contribution of clotting to the accumulation of CREKA-SPIO in tumor vessels was also studied. Quantitative analysis of tumor magnetization with a Superconducting Quantum Interference Device (SQUID) (FIG. 4A) and measurement of the fluorescence signal revealed about 6-fold greater accumulation of CREKA-SPIO in Ni-liposome-pretreated mice compared to PBS-pretreated mice. Aminated SPIO control particles did not significantly accumulate in the tumors (FIG. 4A).

The SQUID measurements revealed that injecting heparin, which is a strong clotting inhibitor, prior to injection of CREKA-SPIO, reduced tumor accumulation of nanoparticles by more than 50% (FIG. 4A). Microscopy showed that heparin reduced the fibrin-positive/CREKA-SPIO positive structures within tumor vessels, but that the particles still bound along the walls of the vessels, presumably to preexisting fibrin deposits (a representative image is shown in FIG. 4B). Separate quantification of the homing pattern showed that heparin did not significantly reduce the number of vessels with nanoparticles bound to the vessel walls, but essentially eliminated the intravascular clotting (FIG. 4C). Thus, the binding of CREKA-SPIO to tumor vessels does not require the clotting activity that is associated with these particles, but clotting improves the efficiency of the tumor homing.

The clotting induced by CREKA-SPIO caused a particularly strong enhancement of tumor signal in whole-body scans. CREKA-SPIO nanoparticles labeled with Cy7, a near infrared fluorescent compound, effectively accumulated in tumors (FIG. 4D, image on the left, arrow), with a significant signal from the liver as well (arrowhead). The reduction in the tumor signal obtained with heparin (FIG. 4D, image on the right) appeared greater in the fluorescence measurements than the 50% value determined by SQUID, possibly because the concentrated signal from the clots enhanced optical detection of the fluorescence. These results show that the clotting induced by CREKA-SPIO provides a particular advantage in tumor imaging.

2. Discussion

This example describes an example of a nanoparticle system that provides effective accumulation of the particles in tumors. The system is based on four elements: First, coating of the nanoparticles with a tumor-homing peptide that binds to clotted plasma proteins endows the particles with a specific affinity for tumor vessels (and tumor stroma). Second, decoy particle pretreatment prolongs the blood half-life of the particles and increases tumor targeting. Third, the tumor-targeted nanoparticles cause intravascular clotting in tumor blood vessels. Fourth, the intravascular clots attract more nanoparticles into the tumor, amplifying the targeting.

A peptide with specific affinity for clotted plasma proteins was chosen as the targeting element for the nanoparticles. The interstitial spaces of tumors contain fibrin and proteins that become cross-linked to fibrin in blood clotting, such as fibronectin (Dvorak 1985; Pilch 2006). The presence of these products in tumors, but not in normal tissues, can be a result of leakiness of tumor vessels, which allows plasma proteins to enter from the blood into tumor tissue, where the leaked fibrinogen is converted to fibrin by tissue procoagulant factors (Dvorak 1985; Abe 1999). The clotting creates new binding sites that can be identified and accessed with synthetic peptides (Pilch 2006). The present results show that the CREKA-modified nanoparticles not only bind to blood and plasma clots, but can also induce localized tumor clotting. The nature of the particle is not limited for this activity, as it was found that both CREKA-coated iron oxide and micron-sized CREKA-coated liposomes cause clotting in tumor vessels. The binding of one or more clotting products by the CREKA-modified particles can shift the balance of clotting and clot dissolution in the direction of clot formation, and the presence of this activity at the surface of particles can facilitate contact-dependent coagulation.

Some nanomaterials are capable of triggering systemic thrombosis (Gorbet 2004), but here the thrombosis induced by the CREKA particles was confined to tumor vessels. The high concentration of the targeted particles in tumor vessels can explain the selective localization of the thrombosis to tumor vessels. However, since no detectable clotting was seen in the liver, where the nanoparticles also accumulate at high concentrations, other factors must be important. The pro-coagulant environment common in tumors can be a major factor contributing to the tumor specificity of the clotting (Boccaccio 2005).

A major advantage of nanoparticles is that multiple functions can be incorporated onto a single entity. Described herein is an in vivo function for nanoparticles; self-amplifying tumor homing enabled by nanoparticle-induced clotting in tumor vessels and the binding of additional nanoparticles to the clots. This nanoparticle system combines several other functions into one particle: specific tumor homing, avoidance of the RES, and effective tumor imaging. Optical imaging was used in this work, but the IO platform also enables MRI imaging. The clotting caused by CREKA-SPIO nanoparticles in tumor vessels serves to focally concentrate the particles in a manner that appears to improve tumor detection by microscopic and whole-body imaging techniques.

Another function of the targeted particles is that they cause physical blockade of tumor vessels by local embolism. Blood vessel occlusion by embolism or clotting can reduce tumor growth (Huang 1997; El-Sheikh 2005). To date, a 20% occlusion rate in tumor vessels has been achieved. Due to the modular nature of nanoparticle design, the functions described herein can be incorporated into particles with additional activities. Drug-carrying nanoparticles that accumulate in tumor vessels and slowly release the drug payload while simultaneously occluding the vessels can be used with the methods and compositions disclosed herein.

3. Materials and Methods

Phage screening, tumors and peptides. In vivo screening of a peptide library with the general structure of CX₇C (SEQ ID NO: 4), where C is cysteine and X is any amino acid, was carried out as described (Oh 2004) using 65- to 75-day-old transgenic MMTV PyMT mice (Hutchinson 2000). These mice express the polyoma virus middle T antigen (MT) under the transcriptional control of the mouse mammary tumor virus (MMTV), leading to the induction of multi-focal mammary tumors in 100% of carriers. MDA-MB-435 tumors in nude mice and peptide synthesis have been described (Laakkonen 2002; Laakkonen 2004). B16F1 murine melanoma tumors were grown in fibrinogen null mice (Suh 1995) and their normal littermates and used when they reached 0.5-1 cm in size (Pilch 2006).

Nanoparticles and liposomes. Amino group-functionalized dextran-coated superparamagnetic iron oxide nanoparticles (50 nm nanomag-D-SPIO; Micromod Partikeltechnologie GmbH, Rostock, Germany) were coupled with CREKA peptide using a crosslinker. The final coupling ratio was 30 nmol fluorescein-labeled peptide molecules per mg iron oxide, or 8,000 peptides/particle. For near-infrared labeling with Cy7, about 20% of the amines were derivatized with Cy7-NHS ester (GE Healthcare Bio-Sciences, Piscataway, N.J.), and the remaining amines were used for conjugating the peptide. Detail on the SPIO and the preparation of liposomes are described below. Clodronate was purchased from Sigma and incorporated into liposomes as described (Van Rooijen and Sanders (1994)).

Nanoparticle injections. For intravenous injections, the animals were anesthetized with intraperitoneal Avertin, and liposomes (2 μmol DSPC) and/or nanoparticles (1-4 mg Fe/kg body weight) were injected into the tail vein. The animals were sacrificed 5-24 h post-injection by cardiac perfusion with PBS under anesthesia, and organs were dissected and analyzed for particle homing. To suppress liver macrophages, mice were intravenously injected with liposomal clodronate suspension (100 μl per mouse), and the mice were used for experiments 24 hours later.

Phage and nanoparticle binding to clots. Phage binding to clotted plasma proteins was determined as described (Pilch 2006). CREKA-SPIO and control SPIO were added to freshly formed plasma clots in the presence or absence of free CREKA peptide. After 10 min incubation, the clots were washed 4 times in PBS, transferred to a new tube and digested in 100 μl concentrated nitric acid. The digested material was diluted in 2 ml distilled water and the iron concentration was determined using inductively coupled plasma—optical emission spectroscopy (ICP-OES, PerkinElmer, Norwalk, Conn.).

Nanoparticle preparation. When necessary to achieve high peptide coupling density, additional amino groups were added to commercially obtained SPIO as follows: First, to crosslink the particles before the amination step, 3 ml of the colloid (˜10 mgFe/ml in double-distilled water) was added to 5 ml of 5M NaOH and 2 ml of epichlorohydrin (Sigma, St. Louis, Mo.). The mixture was agitated for 24 hours at room temperature to promote interaction between the organic phase (epichlorohydrin) and aqueous phase (dextran-coated particle colloid). In order to remove excess epichlorohydrin, the reacted mixture was dialyzed against double-distilled water for 24 hours using a dialysis cassette (10,000 Da cutoff, Pierce, Rockford Ill.). Amino groups were added to the surface of the particles as follows: 0.02 ml of concentrated ammonium hydroxide (30%) was added to 1 ml of colloid (˜10 mg Fe/ml). The mixture was agitated at room temperature for 24 hours. The reacted mixture was dialyzed against double-distilled water for 24 hours. To further rinse the particles, the colloid was trapped on a MACS® Midi magnetic separation column (Miltenyi Biotec, Auburn Calif.), rinsed with PBS three times, and eluted from the column with 1 ml PBS.

To conjugate CREKA peptide to SPIO, the particles were re-suspended at a concentration of 1 mg Fe/ml, and heterobifunctional linker N4a-maleimidoacetoxy]succinimide ester (AMAS; Pierce) was added (2.5 mg linker per 2 mg Fe) under vortexing. After incubation at room temperature for 40 min, the particles were washed 3 times with 10 ml PBS on a MACS column. The peptide with free terminal cysteine was then added (100 μg peptide per 2 mg Fe). After incubation overnight at 4° C. the particles were washed again and re-suspended in PBS at a concentration of 0.35 mg/ml of Fe). To quantify the number of peptide molecules conjugated to the particles, a known amount of stock or AMAS-activated particles was incubated with varying amounts of the peptide. After completion of the incubation the particles were pelleted at 100.000 G using Beckman TLA 100.3 ultracentrifuge rotor (30 min) and the amount of the unbound peptide was quantified by fluorescence. To cleave the conjugated peptide from the particles, the particles were incubated at 37° C. overnight at pH 10. The concentration of free peptide in the supernatant was determined by reading fluorescence and by using the calibration curve obtained for the same peptide. The fluorescence intensity of known amounts of particles was plotted as a function of peptide conjugation density, and the slope equation was used to determine conjugation density in different batches.

Liposome preparation. To prepare liposomes, 1,2-Distearoyl-sn-glycero-3-phosphocholine (DSPC), cholesterol, and 1,2-Dioleoyl-sn-glycero-3-{[N(5-amino-1-carboxypentyl) iminodiacetic acid]succinyl} (nickel salt) (all from Avanti Polar Lipids, Alabaster Ala.), were mixed in chloroform at a molar ratio of 57:37:10 and evaporated in a rotary evaporator until dry. The lipids were hydrated in PBS to a final DSPC concentration 10 mM. The lipid mixture was extensively bath sonicated for 10 min at 55° C. to facilitate liposome formation. For plain liposomes only DSPC and cholesterol were used at a molar ratio of 57:37.

CREKA-decorated liposomes were prepared by reacting PEG-DSPE-maleimide (Avanti) with a 2-fold molar excess of CREKA. The reaction was performed at room temperature under nitrogen in PBS buffer, pH 7.4. After the reaction had been completed in 2 hours, the product (yellow precipitate) was washed by centrifugation and dissolved in ethanol. The ethanol solution was stored at −20° C. CREKA-PEG was incorporated by adding a liposome suspension to a dried film of CREKA-PEG-DSPE, heating to 55° C. while vortexing for 1 hour. Control liposomes were prepared as above but using FITC-PEG-DSPE instead. The liposome preparations were kept at 4° C. until used.

Analysis of protein binding by nanoparticles. To test the binding of soluble plasma proteins to SPIO nanoparticles, the particles were incubated with citrated mouse plasma at a concentration of 1-2 mg iron/ml plasma. Alternatively, the particles were injected into animals and plasma was collected 5-10 min post-injection. The particles were washed on the magnetic column to remove non-bound proteins, and the particles were boiled in 10% SDS for 20 min. The iron oxide was pelleted by ultracentrifugation (100.000 g, 10 min) and the eluted proteins in the supernatant were precipitated with acetone overnight at −20° C. The protein pellet was analyzed by SDS-PAGE, and the gels were silver stained (SilverQuest, Invitrogen, Carlsbad, Calif.). For mass spectrometric analysis, proteins extracted from the particles were reconstituted in water; a protein aliquot was digested with trypsin and analyzed using Applied Biosystems PE SCIEX QSTARR liquid chromatograph Q-TOF mass spectrometer, Foster City, Calif. The data were analyzed using Mascot search engine (Matrix Science, Boston, Mass.).

Nanoparticle clearance. Heparinized capillaries were used to draw 50 μl of blood from the periorbital plexus at different times after nanoparticle injection, the blood was centrifuged at 300 g for 2 min, and a 10 μl aliquot of platelet-rich plasma was diluted into 600 μl 1M Tris solution, pH 8.4. Fluorescence was determined on a PerkinElmer (Norwalk, Conn.) LS50B spectrofluorometer, and plotted as a function of the time the particles had circulated.

Intravital microscopy. Tumor blood flow in MDA-MB 435 xenograft-bearing mice was observed by intravital microscopy. Mice were pre-injected with Ni-liposomes and 5×10⁸ of DiI-labeled erythrocytes. A skin flap was moved aside to expose the tumors, and the mice were intravenously injected with 4 mg/kg of fluorescein-CREKA-SPIO (time “0”). The tumors were scanned with IV-100 intravital laser scanning microscope (Olympus Corp., Tokyo, Japan) using an IV-OB35F22W29 MicroProbe objective (Olympus Corp., Tokyo, Japan). Movies were recorded at 10 min intervals up to 120 min post-injection.

Magnetic measurements of the tissue samples using Superconducting Quantum Interference Device (SQUID) magnetometer. Tissue samples were frozen immediately upon collection, lyophilized, weighed, and placed in gelatin capsules. The capsules were inserted into the middle of transparent plastic straws for magnetic measurements made using a Quantum Design MPMS2 SQUID magnetometer (San Diego, Calif.) operated at 150 K. The samples were exposed to direct current magnetic fields in stepwise increments up to one Tesla. Corrections were made for the diamagnetic contribution of tissue, capsule and straw.

### B. Example 2

### Targeting Atherosclerosis Using Modular, Multifunctional Micelles

Subtle clotting that occurs on the luminal surface of atherosclerotic plaques, presents a novel target for nanoparticle-based diagnostics and therapeutics. A multifunctional, modular micelles was developed that contain a targeting element, a fluorophore and, when desired, a drug component in the same particle. Targeting atherosclerotic plaques in ApoE null mice fed a high fat diet was accomplished with the pentapeptide CREKA (SEQ ID NO:1) (cysteine-arginine-glutamic acid-lysine-alanine), which binds to clotted plasma proteins. The fluorescent micelles bind to the entire surface of the plaque and notably, concentrate at the shoulders of the plaque, a location that is prone to rupture. The targeted micelles also deliver an increased concentration of the anticoagulant drug, hirulog, to the plaque when compared to untargeted micelles.

1. Results

Modular, Multifunctional Micelles. The general structure of the micelles is shown in FIG. 10. Individual lipopeptide monomers were made with a 1,2-distearoyl-sn-glycero-3-phosphoethanol-amine (DSPE) tail, a PEG(2000) spacer, and a variable head group, which was either the carboxyfluorescein (FAM)-CREKA peptide, an infrared fluorophor, or the hirulog peptide. When placed in aqueous solution, these compounds formed micelles with an average hydrodynamic diameter of 17.0±1.0 nm. The composition of the micelles can be varied, for instance targeted micelles from the FAM-CREKA monomers alone, or by mixing all three monomers together were made. Non-targeted control micelles were obtained by mixing FAM-labeled monomers with N-acetyl cysteine monomers. Half-life of FAM-CREKA micelles in circulation was determined by fluorescence and was 130 minutes. The half-life in circulation of the fluorescent CREKA/hirulog mixed micelles was determined using anti-thrombin activity and found to be about 90 minutes.

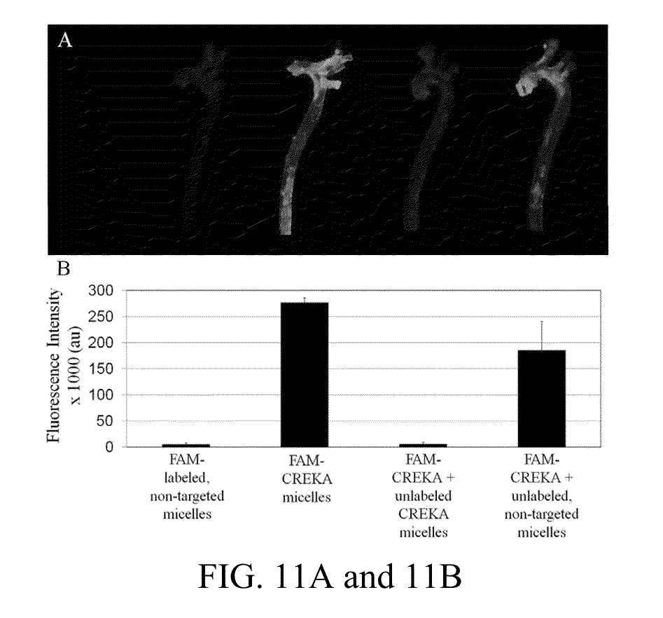

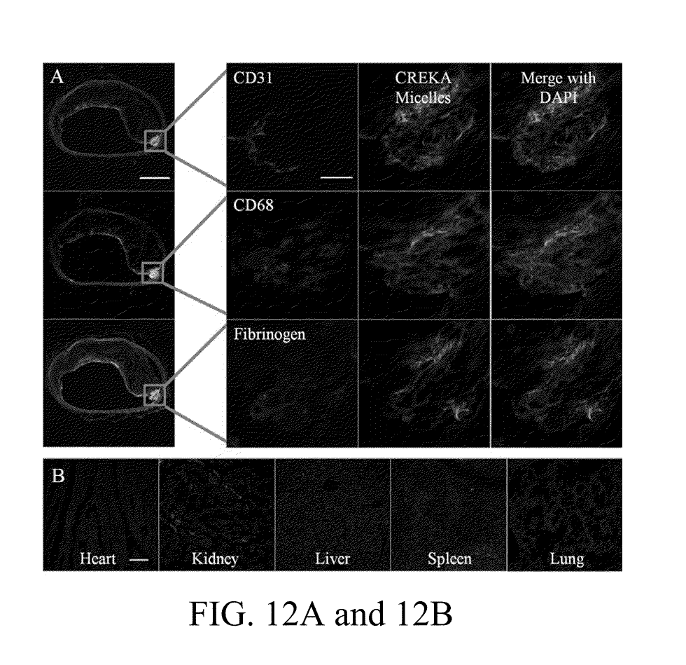

Ex vivo Imaging of the Aortic Tree in Atherosclerotic Mice. Atherosclerotic plaques in ApoE null mice were obtained by keeping the mice on a high fat diet (Nakashima Y, et al., (1994) Arterioscler Thromb 14, 133-140; Reddick R L, et al., (1994) Arterioscler Thromb 14, 141-147). Earlier studies have revealed fibrin accumulation at the surface and interior of atherosclerotic plaques in other animal models and on human plaques (Eitzman D T, et al. (2000) Blood 96, 4212-4215). Similar results are shown in the ApoE model; anti-fibrin(ogen) antibodies stained the plaques, but not normal-appearing vessel wall in this model (see FIG. 12A), indicating the presence of clotted plasma proteins at these sites. These fibrin deposits served as a target for imaging. Fluorescein-labeled CREKA micelles were injected into the mice and imaged the isolated aortic tree ex vivo. High fluorescence intensity was observed in the regions that contained most of the atherosclerotic lesions. In the ApoE null mouse these regions include the brachiocephalic artery and the lower aortic arch (Maeda N, et al., (2007) Atherosclerosis 195, 75-82). Quantitative comparison with fluorescent, non-targeted micelles revealed a large difference between the micelles that were targeted (fluorescence intensity in arbitrary units: 277,000±10,000) and those not targeted (5,100±3,300; FIG. 11). The difference was statistically significant (p≦0.001). The fluorescence in the aortic tree from the CREKA-targeted micelles was abolished when an excess of unlabeled CREKA micelles was pre-injected (5,200±5,000; p≦0.001), whereas unlabeled, non-targeted micelles did not significantly inhibit the CREKA micelle homing (186,000±56,000). These results show that CREKA micelles are able to specifically target the diseased vasculature in atherosclerotic mice and concentrate in areas that are prone to atherosclerotic plaque formation.

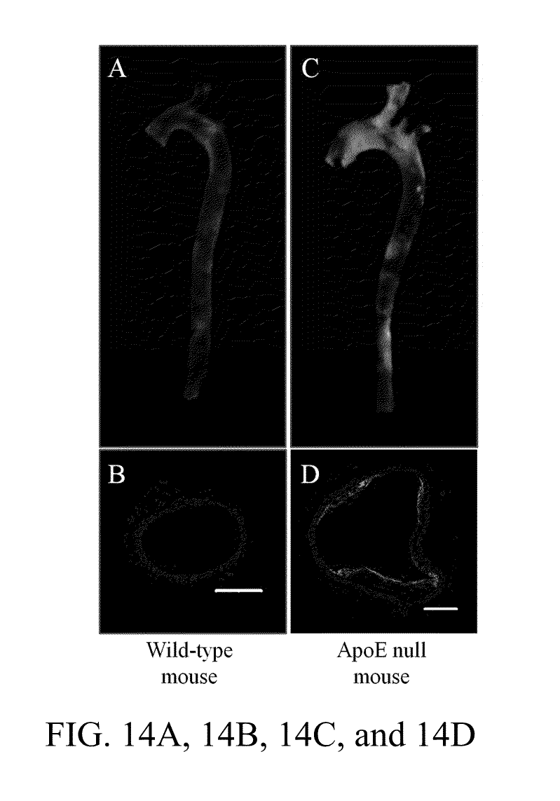

Binding of CREKA Micelles to Atherosclerotic Plaques. Histological examination of the vascular tree from mice injected with CREKA micelles showed fluorescence on the luminal surface of plaques, while there was no significant binding to the histologically healthy portion of the blood vessel in microscopic cross-sections (FIG. 12A). Strikingly, the micelles concentrate in the shoulder regions of the plaque (inset, FIG. 12A) where plaques are known to be prone to rupture (Falk E, et al., (2007) Arterioscler Thromb Vasc Biol 27, 969-972; Richardson P D, et al., (1989) Lancet 2, 941-944). Fluorescence from the micelles was seen underneath the endothelial layer in the plaque in areas of high inflammation as shown with anti-CD31 (endothelial cells) and anti-CD68 (macrophages and lymphocytes) immunofluorescence. Clotted plasma proteins were visualized on the surface of and throughout the interior of the plaque using anti-fibrinogen antibodies. CREKA micelles did not bind substantially to other tissues including the heart and lungs, but small quantities were found in the liver, spleen, and kidneys, tissues known to non-specifically trap nanoparticles (FIG. 12B). Also, there was no accumulation of CREKA micelles in the aortas of normal mice (FIG. 14). Thus, CREKA micelles specifically target atherosclerotic plaques, concentrating in areas that are prone to rupture with no appreciable binding to healthy vasculature.

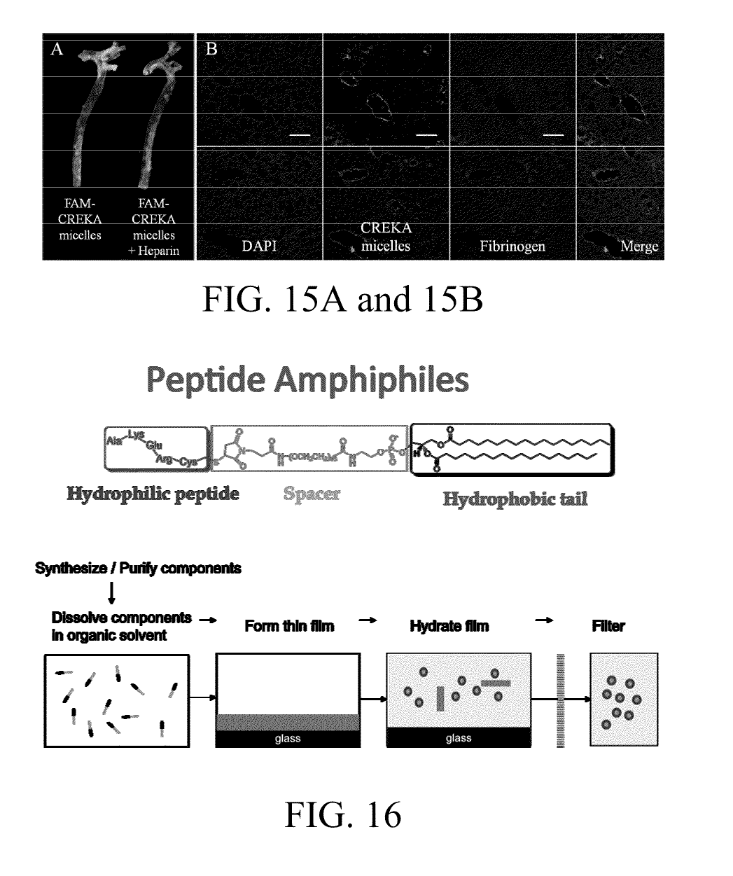

Role of Clotting in Binding of CREKA Micelles to Atherosclerotic Plaques. Binding of CREKA iron oxide nanoparticles to tumor vessels has previously been shown to induce clotting in the lumen of these vessels and amplify the binding of the particles (Simberg D, et al., (2007) Proc Natl Acad Sci USA 104, 932-936). The tumor homing of these was greatly reduced in that study by pre-injecting heparin, which prevented the clotting-induced amplification. The clotting-mediated amplification, while potentially beneficial in the diagnosis and treatment of cancer, would not be desirable in the management of atherosclerosis. No clotting was observed in the lumen of atherosclerotic blood vessels in microscopic cross-sections following injection of CREKA micelles. Furthermore, high fluorescence intensity was still observed in the aortas of atherosclerotic mice injected with FAM-CREKA micelles after a pre-injection of heparin (FIG. 15A). In order to determine if the absence of induction of clotting by CREKA at the plaque surface was a characteristic of the micelles or the plaque microenvironment, CREKA micelles were injected into mice bearing 22RV1 tumors in which CREKA iron oxide nanoparticles cause intravascular clotting. CREKA micelles accumulated at the walls of tumor vessels, but caused no detectable intravascular clotting (FIG. 15B). Thus, unlike CREKA iron oxide particles (Rosamond W, et al., (2007) Circulation 115, e69-171), CREKA micelles do not induce clotting in the target vessels, showing that the CREKA micellar platform is suitable for nanoparticle targeting to atherosclerotic plaques.

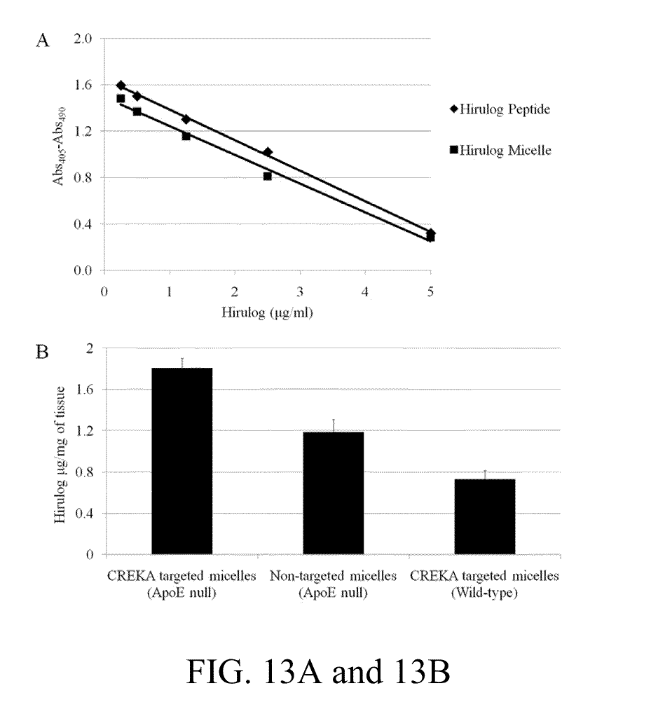

Targeting of the Anti-Thrombin Peptide, Hirulog to Atherosclerotic Plaques. The anticoagulant, heparin, is used in patients with unstable angina to prevent further clots from forming. However, this drug inhibits thrombin indirectly and cannot inhibit the thrombin that is already bound to fibrin. Moreover, its use can also lead to serious complications including major bleeding events and thrombocytopenia. Direct thrombin inhibitors have fewer side effects and can inhibit thrombin that is already bound to a blood clot. Hirulog, a small synthetic peptide, was designed by combining the active sites from the natural thrombin inhibitor, hirudin, through a flexible glycine linker into a single 20-amino acid peptide (Maraganore J M, et al., (1990) Biochemistry 29, 7095-7101.). Hirulog was conjugated with micellar nanoparticles and showed that it retains full activity in a chromogenic assay for thrombin activity (FIG. 13A). CREKA-targeted micelles were used to deliver hirulog to atherosclerotic plaques. CREKA/FAM/hirulog mixed micelles were injected into atherosclerotic mice and allowed to circulate for 3 hours. The accumulation of fluorescence in atherosclerotic aortas was identical to that of CREKA/FAM micelles described above. Anti-thrombin activity in the excised aortic tree was significantly higher in the aortas of mice injected with CREKA targeted micelles than in mice that received non-targeted micelles (1.8 μg/mg and 1.2 μg/mg of tissue, p≦0.05). CREKA targeted micelles also caused significantly higher anti-thrombin activity in the aortas of atherosclerotic than wild type mice (0.8 μg/mg of tissue, p≦0.05, FIG. 13B). This demonstrates that CREKA targeted micelles can selectively deliver hirulog to plaques.

2. Discussion

Targeted micellar nanoparticles can be used to direct compounds and compositions (for example, diagnostic imaging dyes and therapeutic compounds) to atherosclerotic plaques in vivo. Mixed micelles composed of lipid-tailed clot-binding peptide CREKA as a targeting element, a fluorescent dye as a labeling agent and, in some cases, hirulog as an anticoagulant, specifically bound to plaques. The plaques accumulated fluorescence and, when hirulog was included in the micelles, an increased level of anti-thrombin activity was seen in the diseased vessels. The modularity that is characteristic to this micellar nanoparticle platform allows multiple functions to be built into the nanoparticle.

Micelles coated with the CREKA peptide were able to specifically target diseased vasculature in ApoE null mice. The specificity of the targeting was evident from a number of observations. First, fluorescence from the micelles in the aortic tree of atherosclerotic mice localized to known areas of plaque formation and no fluorescence was observed in wild-type mice. Second, CREKA micelles bind to the entire surface of the plaque in histological sections, but do not bind to the healthy portion of the vessel. Third, an excess of unlabeled CREKA micelles inhibited the plaque binding of fluorescent CREKA micelles. Thus, micelles targeted with the CREKA peptide present a potentially useful approach to targeting atherosclerotic plaques.

While the CREKA micelles decorated the entire surface of plaques, the strongest accumulation of the micelles was at the shoulder, the junction between the plaque and the histologically healthy portion of the vessel wall, which are the sites most prone to rupture (Richardson P D, et al., (1989) Lancet 2, 941-944). The high concentration of targeted micelles in the lesion shoulder indicates that these micelles may be effective in delivering compounds to rupture-prone plaques.

Increased fluorescence was observed in the aortic tree of atherosclerotic mice after injection of fluorescent CREKA micelles in imaging. However, CREKA micelles labeled with the infrared dye Cy7 did not produce a sufficient signal to visualize the plaques in vivo, presumably because of insufficient tissue penetration of the exciting and emitted signals. The modularity of the micelles allows the construction of probes for more sensitive and penetrating imaging techniques, such as PET or MRI.

The homing of CREKA-coated iron-oxide nanoparticles to tumors is partially dependent on blood clotting induced by the particles within tumor vessels (Davies M J (1992) Circulation 85, 119-24). Importantly, CREKA micelles are less thrombogenic than CREKA-coated iron oxide nanoparticles because the micelles, while homing to tumor vessels, did not induce any detectable additional clotting in them. Moreover, inhibiting blood clotting in atherosclerotic mice with heparin had no significant effect on the accumulation of CREKA micelles in the plaques. Thus, the thrombogenicity of CREKA micelles is low and they significantly target only preformed clotted material in both tumors and plaques.

Because the presence of the anticoagulant heparin did not significantly reduce CREKA micelle targeting to plaques, CREKA micelles functioned to deliver an anticoagulant to these lesions. Like CREKA/FAM micelles, CREKA/hirulog mixed micelles accumulated in the rupture-prone shoulder regions of plaques and significantly increased anti-thrombin activity in the diseased vasculature. Thus, the CREKA micelle platform can be used reduce the clotting tendency in plaques and can also reduce the risk of thrombus formation upon plaque rupture. Moreover, the targeting makes it possible to lower the dose, which should reduce the risk of bleeding complications.

3. Materials and Methods

Micelles. The anticoagulant peptide hirulog-2 was modified by adding a cysteine residue to the N-terminus (Cys-(D-Phe)-Pro-Arg-Pro-(Gly)4-Asn-Gly-Asp-Phe-Glu-Glu-Ile-Pro-Glu-Glu-Tyr-Leu) for covalent conjugation to the micelle lipid tail. Synthesis of all of the peptides was performed by adapting Fmoc/t-Bu strategy on a microwave assisted automated peptide synthesizer (Liberty, CEM Corporation). Peptide crudes were purified by HPLC using 0.1% TFA in acetonitrile-water mixtures. The peptides obtained were 90%-95% pure by HPLC and were characterized by Q-TOF mass spectral analysis.

1,2-distearoyl-sn-glycero-3-phosphoethanolamine-N-[maleimide(polyethylene glycol)-2000] (DSPE-PEG(2000)-maleimide) and 1,2-distearoyl-sn-glycero-3-phosphoethanolamine-N-[amino(polyethylene glycol)-2000] (DSPE-PEG(2000)-amine) were purchased from Avanti Polar Lipids, Inc. Cy7 mono NHS ester was purchased from Amersham Biosciences.

Cysteine-containing peptides were conjugated via a thioether linkage to DSPE-PEG(2000)-maleimide by adding a 10% molar excess of the lipid to a water:methanol solution (90:10 by volume) containing the peptide. After reaction at room temperature for 4 hours, a solution of N-acetyl cysteine (Sigma) was added to react with free maleimide groups. The resulting product was then purified by reverse-phase, high-performance, liquid-chromatography (HPLC) on a C4 column (Vydac) at 60° C.

Cy7 was conjugated via a peptide bond to DSPE-PEG(2000)-amine by adding a 3-fold molar excess of Cy7 mono NHS ester to the lipid dissolved in 10 mM aqueous carbonate buffer (pH=8.5) containing 10% methanol by volume. After reaction at 4° C. for 8 hours, the mixture was purified by HPLC as above.

Mixtures of fluorophore and peptide-containing DSPE-PEG(2000) amphiphiles were prepared in a glass culture tube by dissolving each pure component in methanol, mixing the components, and evaporating the mixed solution under nitrogen. The resulting film was dried under vacuum for 8 hours then hydrated at 80° C. in water with a salt concentration of 10 mM NaCl. Samples were incubated at 80° C. for 30 minutes and allowed to cool to room temperature for 60 minutes. Solutions were then filtered through a 220 nm poly(vinylidenefluoride) syringe filter (Fisher Scientific).

Micelle Size as Determined by Dynamic Light Scattering. The presence of small, spheroidal micelles was confirmed by particle size measurements using dynamic light scattering (DLS). The DLS system (Brookhaven Instruments) consisted of an avalanche photodiode detector to measure scattering intensity from a 632.8 nm HeNe laser (Melles Griot) as a function of delay time. A goniometer was used to vary measurement angle, and consequently, the scattering wave vector, q.

The first cumulant, F, of the first-order autocorrelation function was measured as a function of scattering wave vector in the range 0.015 to 0.025 nm⁻¹. The quantity, Γ/q², was linearly extrapolated to q=0 to determine the translational diffusion coefficient of the aggregate and the Stokes-Einstein [perhaps a reference for the less physical science inclined] relationship was used to estimate the micelle hydrodynamic diameter based on the measured diffusion coefficient.

Half-life of Micelles in Circulation. The half-life of FAM-CREKA micelles in circulation was determined by injecting 100 μL of 1 mM solution of micelles into Balb/c wild-type mice intravenously. Blood was collected from the retro-orbital sinus with heparinized capillary tubes from the same mouse at various time points post injection. The blood was centrifuged at 1000 g for 2 min, and a 10 μL aliquot of plasma was diluted to 100 μL with PBS. Fluorescence of the plasma was measured using a fluorimeter at an excitation wavelength of 485 nm and emission wavelength of 528 nm.

The half-life of FAM-CREKA/Cy7/hirulog mixed micelles in circulation was determined by injecting 100 μl of 1 mM micelles into C57BL/6 wild-type mice intravenously. Blood was collected in 3.2% buffered sodium citrate at various time points from different mice by cardiac puncture and centrifuged at 1000 g for 10 min. Plasma was then analyzed for anti-thrombin activity using an assay with the S-2366 chromogenic substrate according to the published protocol for hirudin (diaPharma, West Chester, Ohio).

Targeting of Micelles to Atherosclerotic Plaques. Transgenic mice homozygous for the Apoe^{tm1Unc} mutation (Jackson labs, Bar Harbor, Me.) were fed a high fat diet (42% fat, TD88137, Harlan, Madison, Wis.) for 6 months to generate stage V lesions (24) in the brachiocephalic artery and aortic arch. Mice were housed and all procedures were performed according to standards of the University of California, Santa Barbara Institutional Animal Care and Use Committee. The mice were injected intravenously through the tail vein with 100 μl, 1 mM micelles containing either FAM-CREKA or a 1:1 mix of FAM and N-acetyl cysteine as head groups. Micelles were allowed to circulate in the mice for 3 hours and the mice were then perfused with ice cold Dulbecco's Modified Eagle Medium (DMEM) through the left ventricle to remove any unbound micelles. The heart, aortic tree, liver, spleen, lungs, and kidneys were excised and fixed with 4% paraformaldehyde overnight at 4° C. Ex vivo imaging was performed using a 530 nm viewing filter, illumatool light source (Light Tools Research, Encinitas, Calif.) and a Canon XTi DSLR camera. Tissue was then treated with a 30% sucrose solution for 8 hours and frozen in OCT for cryosectioning. Quantification of fluorescence intensity was performed using Image J software.

Tumor Targeting with CREKA Micelles. Orthotopic prostate cancer xenografts were generated by implanting 22Rv-1 (2×10⁶ cells in 30 μl of PBS) human prostate cancer cells, into the prostate gland of male nude mice. When tumor volumes reached approximately 500 mm³, the mice were injected with 100 μl of 1 mM FAM-CREKA micelles intravenously through the tail vein. The micelles were allowed to circulate for 3 hours and then mice were perfused through the left ventricle with ice cold DMEM. The tumor was excised and frozen in OCT for sectioning.

Immunofluorescence. Serial cross-sections 5 μm thick of the brachiocephalic artery, aortic arch, healthy vessel, control organs, or 22Rv-1 prostate tumor were mounted on silane treated microscope slides (Scientific Device Laboratory, Des Plaines, Ill.) and allowed to air dry. Sections were fixed in ice-cold acetone for 5 minutes and then blocked with Image-iT FX signal enhancer (Invitrogen, Carlsbad, Calif.). Alexa Fluor 647 conjugated rat anti-mouse antibodies to CD31 and CD68 (AbD Serotech, Raleigh, N.C.) were used to visualize endothelial cells and macrophages and other lymphocytes, respectively. Fibrinogen was stained with a primary polyclonal antibody made in goat and Alexa Fluor 647 conjugated anti-goat secondary antibody (Invitrogen, Carlsbad, Calif.). Sections were co-stained with DAPI in ProLong Gold antifade mounting medium (Invitrogen, Carlsbad, Calif.). Images of the vessels were taken using a confocal microscope.

Quantification of Hirulog Activity at Plaque Surface. The mice were injected intravenously through the tail vein with 100 μl, 1 mM (total lipid content) mixed micelles containing FAM-CREKA, CREKA, Cy7, and hirulog as head groups in a 3:3:0.3:3.7 ratio, respectively. Micelles were allowed to circulate in the mice for 3 hours and then mice were perfused with ice cold DMEM through the left ventricle to remove any unbound micelles. The aortic tree was excised and homogenized in 1 ml of normal human plasma with sodium citrate (US Biological, Swampscott, Mass.). Hirulog anti-thrombin activity was then quantified using an assay with the S-2366 chromogenic substrate according to the published protocol for hirudin (diaPharma, West Chester, Ohio).

### REFERENCES

Abe K, Shoji M, Chen J, Bierhaus A, Danave I, Micko C, Casper K, Dillehay D L, Nawroth P P, Rickles F R (1999) Proc Natl Acad Sci USA 96: 8663-8668.

Akerman M E, Chan W C, Laakkonen P, Bhatia S N, Ruoslahti E (2002) Proc Natl Acad Sci USA 99: 12617-12621.

Alonso A, Della Martina A, Stroick M, Fatar M, Griebe M, Pochon S, Schneider M, Hennerici M, Allemann E, & Meairs S (2007) Stroke 38, 1508-1514.

Boccaccio C, Sabatino G, Medico E, Girolami F, Follenzi A, Reato G, Sottile A, Naldini L, Comoglio P M (2005) Nature 434: 396-400.

Bode C, Hudelmayer M, Mehwald P, Bauer S, Freitag M, von Hodenberg E, Newell J B, Kubler W, Haber E, & Runge M S (1994) Circulation 90, 1956-1963.

Boucher Y, Baxter L T, Jain, R K (1990) Cancer Res 50: 4478-4484.

Briley-Saebo K C, Shaw P X, Mulder W J, Choi S H, Vucic E, Aguinaldo J G, Witztum J L, Fuster V, Tsimikas S, & Fayad Z A (2008) Circulation 117, 3206-3215.

Cai W, Shin D W, Chen K, Gheysens O, Cao Q, Wang S X, Gambhir S S, Chen X (2006) Nano Lett 6: 669-676.

Davies M J (1992) Circulation 85, 119-24.

DeNardo S J, DeNardo G L, Miers L A, Natarajan A, Foreman A R, Gruettner C, Adamson G N, Ivkov R (2005) Clin Cancer Res 11: 7087s-7092s.

Desai N, Trieu V, Yao Z, Louie L, Ci S, Yang A, Tao C, De T, Beals B, Dykes D, Noker P, Yao R, Labao E, Hawkins M, Soon-Shiong P (2006) Clin Cancer Res 12: 1317-1324.

Duguid J B (1946) J Pathol Bacterial 58, 207-212.

Duguid J B (1948) J Pathol Bacterial 60, 57-61.

Dvorak H F, Senger D R, Dvorak A M, Harvey V S, McDonagh J (1985) Science 227: 1059-1061.

Eitzman D T, Westrick R J, Xu Z, Tyson J, & Ginsburg D (2000) Blood 96, 4212-4215.

El-Sheikh A, Borgstrom P, Bhattacharjee G, Belting M, Edgington T S (2005) Cancer Res 65: 11109-11117.

Falk E, Schwartz S M, Galis Z S, & Rosenfeld M E (2007) Arterioscler Thromb Vasc Biol 27, 969-972.

Fernandez-Urrusuno R, Fattal E, Rodrigues J M, Jr, Feger J, Bedossa P, Couvreur P (1996) J Biomed Mater Res 31: 401-408.

Ghigliotti G, Waissbluth A R, Speidel C, Abendschein D R, & Eisenberg P R (1998) Arterioscler Thromb Vasc Biol 18, 250-257.

Gorbet M B, Sefton M V (2004) Biomaterials 25: 5681-5703.

Hoffman J A, Giraudo E, Singh M, Zhang L, Inoue M, Porkka K, Hanahan D, Ruoslahti E (2003) Cancer Cell 4: 383-391.

Houston P, Goodman J, Lewis A, Campbell C J, & Braddock M (2001) FEBS Lett 492, 73-77.

Huang X, Molema G, King S, Watkins L, Edgington T S, Thorpe P E (1997) Science 275: 547-550.

Hutchinson J N, Muller W J (2000) Oncogene 19: 6130-6137.

Hutchinson J N, Muller W J (2000) Oncogene 19: 6130-6137.

Ivey M E & Little P J (2008) Thromb Res.

Jung C W, Jacobs P (1995) Magn Reson Imaging 13: 661-674.

Jung, C W (1995) Magn Reson Imaging 13: 675-691.

Karmali P P, Kotamraju V R, Kastantin M, Black M, Missirlis D, Tirrell M, & Ruoslahti E (2008) Nanomedicine.

Kelly K A, Nahrendorf M, Yu A M, Reynolds F, & Weissleder R (2006) Mol Imaging Biol 8, 201-207.

Khandoga A, Stampfl A, Takenaka S, Schulz H, Radykewicz R, Kreyling W, Krombach F (2004) Circulation 109: 1320-1325.

Laakkonen P, Akerman M E, Biliran H, Yang M, Ferrer F, Karpanen T, Hoffman R M, Ruoslahti E (2004) Proc Natl Acad Sci USA 101: 9381-9386.

Laakkonen P, Porkka K, Hoffman J A, Ruoslahti E (2002) Nat Med 8: 751-755.

Liu C, Bhattacharjee G, Boisvert W, Dilley R, & Edgington T (2003) Am J Pathol 163, 1859-1871.

Maeda N, Johnson L, Kim S, Hagaman J, Friedman M, & Reddick R (2007) Atherosclerosis 195, 75-82.

Maraganore J M, Bourdon P, Jablonski J, Ramachandran K L, & Fenton J W, 2nd (1990) Biochemistry 29, 7095-7101.

Moghimi S M, Hunter A C, Murray J C (2001) Pharmacol Rev 53: 283-318.

Moore A, Weissleder R, Bogdanov A, Jr (1997) J Magn Reson Imaging 7: 1140-1145.

Nakashima Y, Plump A S, Raines E W, Breslow J L, & Ross R (1994) Arterioscler Thromb 14, 133-140.

Oh P, Li Y, Yu J, Dun E, Krasinska K M, Carver L A, Testa J E, Schnitzer J E (2004) Nature 429: 629-635.

Pasqualini R, Ruoslahti E (1996) Nature 380: 364-366.

Pilch J, Brown D M, Komatsu M, Jarvinen T A, Yang M, Peters D, Hoffman R M, Ruoslahti E (2006) Proc Natl Acad Sci USA 103: 2800-2804.

Radomski A, Jurasz P, Alonso-Escolano D, Drews M, Morandi M, Malinski T, Radomski M W (2005) Br J Pharmacol 146: 882-93.

Reddick R L, Zhang S H, & Maeda N (1994) Arterioscler Thromb 14, 141-147.

Richardson P D, Davies M J, & Born G V (1989) Lancet 2, 941-944.

Rosamond W, Flegal K, Friday G, Furie K, Go A, Greenlund K, Haase N, Ho M, Howard V, Kissela B, et al. (2007) Circulation 115, e69-171.

Ruoslahti, E (2002) Nat Rev Cancer 2: 83-90.

Simberg D, Duza T, Park J H, Essler M, Pilch J, Zhang L, Derfus A M, Yang M, Hoffman R M, Bhatia S, et al. (2007) Proc Natl Acad Sci USA 104, 932-936.

Sinek J, Frieboes H, Zheng X, Cristini V (2004) Biomed Microdevices 6: 297-309.

Smith E B (1993) Wien Klin Wochenschr 105, 417-424.

Souhami R L, Patel H M, Ryman B E (1981) Biochim Biophys Acta 674: 354-371.

Stoll P, Bassler N, Hagemeyer C E, Eisenhardt S U, Chen Y C, Schmidt R, Schwarz M, Ahrens I, Katagiri Y, Pannen B, et al. (2007) Arterioscler Thromb Vasc Biol 27, 1206-1212.

Suh T T, Holmback K, Jensen N J, Daugherty C C, Small K, Simon D I, Potter S, Degen J L (1995) Genes Dev 9: 2020-2033

Van der Heyde H C, Gramaglia I, Sun G, Woods C (2005) Blood 105: 1956-1963.

Van Rooijen N, Sanders A (1994) J Immunol Methods 174: 83-93.

Van Rooijen N, Sanders A (1994) J Immunol Methods 174: 83-93.

Weissleder R, Bogdanov A, Jr, Neuwelt E A, Papisov M (1995) Advanced Drug Delivery Reviews 16: 321-334.

Weissleder R, Stark, D D, Engelstad B L, Bacon B R, Compton C C, White D L, Jacobs P, Lewis J (1989) AJR Am J Roentgenol 152: 167-173.

Whitman S C (2004) Clin Biochem Rev 25, 81-93.

### SEQUENCES

SEQ ID NO:1 CREKA

SEQ ID NO:2 CGLIIQKNEC

SEQ ID NO:3 CNAGESSKNC

SEQ ID NO:4 CXXXXXXXC, where C is cysteine and X is any amino acid

SEQ ID NO:5 CRKDKC

SEQ ID NO:6 CARSKNKDC

1. A composition comprising amphiphile molecules, wherein at least one of the amphiphile molecules comprises a clot-binding head group, wherein the clot-binding head group selectively binds to clotted plasma protein, and wherein the composition does not cause clotting.

2. The composition of claim 1, wherein at least one of the amphiphile molecules comprises a functional head group.

3. The composition of claim 2, wherein the functional head group is a detection head group.

4. The composition of claim 2, wherein the functional head group is a treatment head group.

5. The composition of claim 1, wherein at least one of the amphiphile molecules comprises a detection head group, and wherein at least one of the amphiphile molecules comprises a treatment head group.

6. The composition of claim 1, wherein the amphiphile molecules were subjected to a hydrophilic medium.

7. The composition of claim 6, wherein the amphiphile molecules formed an aggregate in the hydrophilic medium.

8. The composition of claim 7, wherein the aggregate comprises a micelle.

9. The composition of claim 1, wherein the clot-binding head group comprises amino acid segments independently selected from amino acid segments comprising the amino acid sequence CREKA (SEQ ID NO: 1) or a conservative variant thereof, amino acid segments comprising the amino acid sequence CREKA (SEQ ID NO:1), amino acid segments consisting of the amino acid sequence CREKA (SEQ ID NO:1), or amino acid segments consisting of the amino acid sequence REK.

10. The composition of claim 9, wherein the amino acid segments each independently comprise the amino acid sequence CREKA (SEQ ID NO: 1) or a conservative variant thereof.

11. The composition of claim 9, wherein the amino acid segments each independently comprise the amino acid sequence CREKA (SEQ ID NO:1).

12. The composition of claim 9, wherein at least one of the amino acid segment consists of the amino acid sequence CREKA (SEQ ID NO:1).

13. The composition of claim 9, wherein at least one of the amino acid segment consists of the amino acid sequence REK.

14. The composition of claim 1, wherein the amphiphile molecules are detectable.

15. The composition of claim 14, wherein the amphiphile molecules are detectable by fluorescence, PET or MRI.

16. The composition of claim 15, wherein the amphiphile molecules are detectable by fluorescence.

17. The composition of claim 16, wherein the detection head group comprises FAM or a derivative thereof.

18. The composition of claim 4, wherein the treatment head group comprises a compound or composition for treating cardiovascular disease.

19. The composition of claim 4, wherein the treatment head group comprises a compound or composition for treating atherosclerosis.

20. The composition of claim 4, wherein the treatment head group comprises a direct thrombin inhibitor.

21. The composition of claim 4, wherein the treatment head group comprises hirulog or a derivative thereof.

22. The composition of claim 4, wherein the treatment head group comprises a compound or composition for treating cancer.

23. The composition of claim 1 comprising a micelle, wherein the micelle comprises the amphiphile molecules.

24. The composition of claim 1 comprising a liposome, wherein the liposome comprises the amphiphile molecules.

25. A conjugate of the composition of claim 1 and a plaque in a subject.

26. A conjugate of the composition of claim 1 and a tumor in a subject.

27. A method comprising administering a composition to a subject, wherein the composition comprises amphiphile molecules, wherein at least one of the amphiphile molecules comprises a clot-binding head group, wherein the clot-binding head group selectively binds to clotted plasma protein, wherein the composition does not cause clotting, wherein the composition binds to clotted plasma protein in the subject.

28. The method of claim 27, wherein at least one of the amphiphile molecules comprises a functional head group.

29. The method of claim 28, wherein the functional head group is a detection head group.

30. The method of claim 28, wherein the functional head group is a treatment head group.

31. The method of claim 27, wherein at least one of the amphiphile molecules comprises a detection head group, and wherein at least one of the amphiphile molecules comprises a treatment head group.

32. The method of claim 27, wherein the subject is in need of treatment of a disease or condition associated with and/or that produces clotted plasma protein.

33. The method of claim 32, wherein administering the composition treats the disease or condition associated with and/or that produces clotted plasma protein.

34. The method of claim 27, wherein the subject is in need of treatment of cardiovascular disease.

35. The method of claim 34, wherein administering the composition treats the cardiovascular disease.

36. The method of claim 34, wherein the cardiovascular disease is atherosclerosis.

37. The method of claim 27, wherein the subject is in need of treatment of cancer.

38. The method of claim 37, wherein administering the composition treats the cancer.

39. The method of claim 27, wherein the subject is in need of detection, visualization, or both of a disease or condition associated with and/or that produces clotted plasma protein.

40. The method of claim 39 further comprising detecting, visualizing, or both the disease or condition associated with and/or that produces clotted plasma protein.

41. The method of claim 27, wherein the subject is in need of detection, visualization, or both of cardiovascular disease.

42. The method of claim 41 further comprising detecting, visualizing, or both the cardiovascular disease.

43. The method of claim 41, wherein the cardiovascular disease is atherosclerosis.

44. The method of claim 27, wherein the subject is in need of detection, visualization, or both of cancer, a tumor, or both.

45. The method of claim 44 further comprising detecting, visualizing, or both the cancer, tumor, or both.

46. The method of claim 27 further comprising, prior to administering, subjecting the amphiphile molecules to a hydrophilic medium.

47. The method of claim 46, wherein the amphiphile molecules form an aggregate in the hydrophilic medium.

48. The method of claim 47, wherein the aggregate comprises a micelle.

49. The method of claim 27, wherein the clot-binding head group comprises amino acid segments independently selected from amino acid segments comprising the amino acid sequence CREKA (SEQ ID NO: 1) or a conservative variant thereof, amino acid segments comprising the amino acid sequence CREKA (SEQ ID NO:1), amino acid segments consisting of the amino acid sequence CREKA (SEQ ID NO:1), or amino acid segments consisting of the amino acid sequence REK.

50. The method of claim 49, wherein the amino acid segments each independently comprise the amino acid sequence CREKA (SEQ ID NO: 1) or a conservative variant thereof.

51. The method of claim 49, wherein the amino acid segments each independently comprise the amino acid sequence CREKA (SEQ ID NO:1).

52. The method of claim 49, wherein at least one of the amino acid segment consists of the amino acid sequence CREKA (SEQ ID NO:1).

53. The method of claim 49, wherein at least one of the amino acid segment consists of the amino acid sequence REK.

54. The method of claim 27 further comprising, following administering, detecting the amphiphile molecules.

55. The method of claim 54, wherein the amphiphile molecules are detected by fluorescence, PET or MRI.

56. The method of claim 55, wherein the amphiphile molecules are detected by fluorescence.

57. The method of claim 56, wherein the detection head group comprises FAM or a derivative thereof.

58. The method of claim 30, wherein the treatment head group comprises a compound or composition for treating cardiovascular disease.

59. The method of claim 30, wherein the treatment head group comprises a compound or composition for treating atherosclerosis.

60. The method of claim 30, wherein the treatment head group comprises a direct thrombin inhibitor.

61. The method of claim 30, wherein the treatment head group comprises hirulog or a derivative thereof.

62. The method of claim 30, wherein the treatment head group comprises a compound or composition for treating cancer.

63. The method of claim 27, wherein the composition conjugates with a plaque in a subject.

64. The method of claim 27, wherein the composition conjugates with a tumor in a subject.

65. A method of making a composition, the method comprising mixing amphiphile molecules, wherein at least one of the amphiphile molecules comprises a clot-binding head group, wherein the clot-binding head group selectively binds to clotted plasma protein, and wherein the composition does not cause clotting.

66. The method of claim 65 further comprising subjecting the amphiphile molecules to a hydrophilic medium.

67. The composition of claim 66, wherein the amphiphile molecules form an aggregate in the hydrophilic medium.

68. The composition of claim 67, wherein the aggregate comprises a micelle.

69. The method of claim 65, wherein at least one of the amphiphile molecules comprises a functional head group.

70. The method of claim 69, wherein the functional head group is a detection head group.

71. The method of claim 69, wherein the functional head group is a treatment head group.

72. The method of claim 65 65-68, wherein at least one of the amphiphile molecules comprises a detection head group, and wherein at least one of the amphiphile molecules comprises a treatment head group.

73. The method of claim 65, wherein the clot-binding head group comprises amino acid segments independently selected from amino acid segments comprising the amino acid sequence CREKA (SEQ ID NO: 1) or a conservative variant thereof, amino acid segments comprising the amino acid sequence CREKA (SEQ ID NO:1), amino acid segments consisting of the amino acid sequence CREKA (SEQ ID NO:1), or amino acid segments consisting of the amino acid sequence REK.

74. The method of claim 73, wherein the amino acid segments each independently comprise the amino acid sequence CREKA (SEQ ID NO: 1) or a conservative variant thereof.

75. The method of claim 73, wherein the amino acid segments each independently comprise the amino acid sequence CREKA (SEQ ID NO:1).

76. The method of claim 73, wherein at least one of the amino acid segment consists of the amino acid sequence CREKA (SEQ ID NO:1).

77. The method of claim 73, wherein at least one of the amino acid segment consists of the amino acid sequence REK.

78. The method of claim 65, wherein the amphiphile molecules are detectable.

79. The method of claim 78, wherein the amphiphile molecules are detectable by fluorescence, PET or MRI.

80. The method of claim 79, wherein the amphiphile molecules are detectable by fluorescence.

81. The method of claim 80, wherein the detection head group comprises FAM or a derivative thereof.

82. The method of claim 71, wherein the treatment head group comprises a compound or composition for treating cardiovascular disease.

83. The method of claim 71, wherein the treatment head group comprises a compound or composition for treating atherosclerosis.

84. The method of claim 71, wherein the treatment head group comprises a direct thrombin inhibitor.

85. The method of claim 71, wherein the treatment head group comprises hirulog or a derivative thereof.

86. The method of claim 71, wherein the treatment head group comprises a compound or composition for treating cancer.


SEQUENCE LISTING (6 sequences)

SEQ ID NO: 1 - protein, 5 aa, Artificial Sequence
  chemically synthesized; homing peptide
CREKA

SEQ ID NO: 2 - protein, 10 aa, Artificial Sequence
  chemically synthesized; homing peptide
CGLIIQKNEC

SEQ ID NO: 3 - protein, 10 aa, Artificial Sequence
  chemically synthesized; homing peptide
CNAGESSKNC

SEQ ID NO: 4 - protein, 9 aa, Artificial Sequence
  chemically synthesized
CXXXXXXXC

SEQ ID NO: 5 - protein, 6 aa, Artificial Sequence
  chemically synthesized; homing peptide
CRKDKC

SEQ ID NO: 6 - protein, 9 aa, Artificial Sequence
  chemically synthesized; homing peptide
CARSKNKDC